(19) United States
(12) Patent Application Publication
(10) Pub. No.: US 2008/0102046 A1
(43) Pub. Date: May 1, 2008

(54) Cosmetic composition

(75) Inventors: Ludovic Thevenet, Bourg la Reine (FR); Gaetan Chevalier, Boigny sur Bionne (FR); Frank Girier-Dufournier, Paris (FR)

Correspondence Address: OLIFF & BERRIDGE, PLC, P.O. BOX 320850, ALEXANDRIA, VA, 22320-4850, US

(73) Assignee: L'OREAL, PARIS (FR)

(21) Appl. No.: 11/878,079

(22) Filed: Jul. 20, 2007

Related U.S. Application Data
(60) Provisional application No. 60/836,690, filed on Aug. 10, 2006.
(60) Provisional application No. 60/836,692, filed on Aug. 10, 2006.
(60) Provisional application No. 60/837,938, filed on Aug. 16, 2006.
(60) Provisional application No. 60/837,908, filed on Aug. 16, 2006.
(60) Provisional application No. 60/837,920, filed on Aug. 16, 2006.
(60) Provisional application No. 60/837,939, filed on Aug. 16, 2006.
(60) Provisional application No. 60/837,940, filed on Aug. 16, 2006.
(60) Provisional application No. 60/838,141, filed on Aug. 17, 2006.
(60) Provisional application No. 60/838,140, filed on Aug. 17, 2006.

(30) Foreign Application Priority Data
Jul. 21, 2006 (FR) 06 06674
Jul. 21, 2006 (FR) 06 06669
Jul. 21, 2006 (FR) 06 06672
Jul. 21, 2006 (FR) 06 06665
Jul. 21, 2006 (FR) 06 06659
Jul. 21, 2006 (FR) 06 06661
Jul. 21, 2006 (FR) 06 06658
Jul. 21, 2006 (FR) 06 06664
Jul. 21, 2006 (FR) 06 06663

(51) Int. Cl. A61K 47/02; A61K 8/18; A61Q 1/08; A61Q 3/02

(52) U.S. Cl. 424/61; 424/63; 514/769

(57) ABSTRACT

The present invention relates to a cosmetic composition comprising: a cosmetically acceptable medium containing at least one aqueous phase,
at least one interference pigment dispersed in this aqueous phase, capable of generating, when the composition is applied to a support, overbrightness points with an intensity of greater than or equal to 3500 cd·m⁻² and with a dominant wavelength of between 580 and 650 nm.

This non provisional application claims the benefit of French Applications Nos. 06 06674, 06 06669, 06 06672, 06 06665, 06 06659, 06 06661, 06 06658, 06 06664, 06 06663 filed on Jul. 21, 2006 and of U.S. Provisional Applications Nos. 60/837,908, 60/837,938, 60/837,920, 60/837,939, 60/837,940 filed on Aug. 16, 2006 and Nos. 60/836,690, 60/836,692 filed on Aug. 10, 2006 and Nos. 60/838,141 and 60/838,140, filed on Aug. 17, 2006.

The present invention relates to cosmetic compositions and more particularly compositions intended for making up the skin, the lips or the integuments.

The invention more particularly relates to cosmetic compositions of red colour. This colour may be obtained conventionally by means of one or more dyes or by means of pigments that produce light via an absorption phenomenon. One drawback of these pigments is that they do not produce a colour that is as strong as desired.

A red colour may also be produced, just like other colours, with an interference pigment via a phenomenon of interference by reflection of light on a multilayer structure comprising a stack of layers whose refractive indices and thicknesses are appropriately chosen, for example a silica core coated with a surface layer of iron oxide.

However, the tolerance on the refractive indices and the thicknesses of the deposited layers is lower for red than for other colours, given the order of appearance of colours during the decomposition of the spectrum of white light.

Furthermore, in the case of the interference pigment with a layer of iron oxide, mentioned above, the red colour produced by the interference phenomenon readily enters into competition with that produced by absorption by the surface layer, which makes the colour finally observed sensitive to the observation conditions and to the environment of the pigment.

The invention is directed towards proposing a composition with a very saturated and very bright red colour, and it achieves this by means of a cosmetic composition comprising:
a cosmetically acceptable medium containing at least one aqueous phase,
at least one interference pigment dispersed in this aqueous phase, capable of generating overbrightness points with an intensity of greater than or equal to 3500 cd·m⁻² and with a dominant wavelength of between 580 and 650 nm.

The presence of an aqueous medium makes it possible to have a lower refractive index around the particles with an interference pigment, which makes it possible to increase the brightness generated by the interference pigment particles.

The optical thickness (product of the thickness of the layer producing the interference by the index of refraction) of the red color interference pigment may range from 310 nm to 430 nm for interference of order 1 and from 620 nm to 860 nm for interference of order 2. These optical thickness cover the red color (from 620 nm to 700 nm) for two orders of interface by taking into account a variation of the angle from 0 to 70° for a cosmetic medium having a refraction index ranging for example from 1.4 to 1.5.

In one of its aspects, independently of what precedes, the invention provides a cosmetic composition comprising, dispersed in a cosmetically acceptable medium, a red interference pigment that is capable of creating highlights with a dominant wavelength in the range 580 nm to 650 nm and with an intensity of 3500 cd·m⁻² or more when the composition is applied to a surface, the composition not containing, in the medium, white fillers or solid bodies that generate a color by absorption, or, when the composition does contains them, the total amount of such solid bodies being 1% or less by weight relative to the total weight of the composition.

This allows the color produced by the interference phenomenon to be clearly dominating compared to the color produced by adsorption and a bright red make-up may be obtained. In this aspect, the composition need not contain white fillers or diffusing pigments in the medium.

Moreover, the kind and the quantity of solid bodies other than the red interference pigment could be a function of the desired optical properties and textures, provided that the interference phenomenon responsible for the red highlights is not in any way deleteriously affected.

In another one of its aspects, independently of what precedes, the invention provides a cosmetic composition comprising, dispersed in a cosmetically acceptable medium:
at least one red interference pigment that is capable of creating highlights with a dominant wavelength in the range 580 nm to 650 nm and with an intensity of 3500 cd·m⁻² or more when the composition is applied to a surface,—reflective particles that are capable of generating, on said surface, other highlights with an intensity that is greater than or equal to the intensity of the red interference pigment, better greater than or equal to 4 000 cd m².

This allows modifying the aspect of the composition without affecting the red color produced by the red interference pigment.

In particular, the above-mentioned reflective particles can be used in a relatively small amount while making it possible, by means of their reflectivity, to modify the clarity of the composition. In addition, reflective particles absorb less light than conventional diffusing pigments that generate a color by an absorption phenomenon.

In another one of its aspects, independently of what precedes, the invention provides a cosmetic composition comprising, in a cosmetically acceptable medium, a red interference pigment that, when the composition is applied to a support, is capable of generating highlights with an intensity of 3000 cd·m⁻² or more and with a dominant wavelength in the range 580 nm to 650 nm, the composition presenting a turbidity index of 100 nephelometric turbidity units (NTU) or less. This allows the color produced by the interference phenomenon to be clearly dominating compared to the color produced by adsorption for precise conditions of observation. When those conditions change, the color produced by adsorption can be observed by the observer.

In another one of its aspects, the invention provides a set of at least two cosmetic compositions comprising, dispersed in a cosmetically acceptable medium, at least one red interference pigment that, when the corresponding composition is applied to a surface, is capable of generating highlights with an intensity of 3000 cd·m⁻² or more and with a dominant wavelength in the range 580 nm to 650 nm, the saturation difference between two compositions of the set being 2 or less, the red interference pigment in said two compositions being at concentrations that differ by at least 1%.

The set may comprise more than two compositions and the above relationship may be satisfied, where appropriate, for any two compositions of the set or for only some of them.

Such a set of compositions makes it possible to have different concentrations of red highlights, and the Applicant has observed, in unexpected manner, that the presence of such an interference pigment having different concentrations does not lead to a significant modification in saturation.

The compositions can have substantially the same medium.

The term “substantially the same medium” means that the same compounds are found in the compositions, at concentrations that can vary as a function of the amount of red interference pigment.

Thus, the content of a compound may differ from one composition to another in order to compensate for the variation in the amount of red interference pigment.

The compositions need not include solid bodies other than the red interference pigment.

Between the two above-mentioned compositions of the set, the amount of red interference pigment can differ by at least 2%.

In what follows, the expression “the composition” may refer to any one of the compositions of the set.

In another of its aspects, the invention provides a cosmetic composition comprising, in a cosmetically acceptable medium:
an interference pigment that is red and that is capable of generating highlights with an intensity that is greater than or equal to 3000 cd·m⁻² and with a dominant wavelength in the range 580 nm to 650 nm; and
magnetic bodies presenting non-zero magnetic susceptibility.

The invention exploits the very particular sensitivity of the red interference pigment to its environment. Thus, by means of the presence of the interference pigment, even a small modification to the orientation and/or to the positioning of the magnetic bodies in the composition is likely, in the invention, to lead to an observable visual effect, e.g. a variation in the intensity and/or in the concentration of the highlights, in particular by means of the red interference pigment being masked to a greater or lesser extent by the magnetic bodies.

The composition can take on a state that prevents any new change in the orientation of the magnetic bodies under the effect of a magnetic field after a given drying time. This applies to a nail varnish, for example.

Alternatively, in some cases, the orientation of the magnetic bodies can be modified at any time, in particular when the composition does not dry or presents a drying time that is very long. This may apply to a foundation, for example.

By way of example, the magnetic field is exerted a short time after depositing the composition, so as to change its appearance before it dries.

Where appropriate, the magnetic bodies can be constituted by the red interference pigment, when said pigment presents non-zero magnetic susceptibility.

In another of its aspects, the invention provides a cosmetic composition comprising, dispersed in a cosmetically acceptable medium:
an interference first pigment that is red and that, when the composition is applied to a surface, is capable of generating red highlights with an intensity of 3000 cd·m⁻² or more and with a dominant wavelength λ₁ in the range 580 nm to 650 nm; and
a reflective second pigment that is silvery or that is colored with a dominant wavelength λ₂ such that |λ₁−λ₂|≧50 nm, this second pigment having an average size that is 30 μm or more, better 40 μm.

The second pigment may be an interference pigment.

The applicant noted that the second pigment can bring new color effects while making it possible for the composition to preserve the intensity of brightness of the red interference pigment, the first and second pigments being able to create, to some extent, a coloured mosaic.

A difficulty can appear in the formulation of the composition when it is wanted to have intensities of highlights of the same order for the red interference pigment and the colored reflective pigments, in order to obtain an effect of relatively homogeneous pixellisation in intensity.

When the coloured reflective pigments have a multi-layer structure, it can be advantageous to use a red interference pigment and coloured reflective pigments having the same heart, because that can make it possible to more easily obtain the same surface quality, which strongly influences the intensity of highlights.

The use of the same heart can also make it possible to more easily obtain the same color generated by absorption when the red interference pigment and the colored reflective pigments present a surfacing carried out in same material, which can be interesting so that the red interference pigment and the colored reflective pigments appear with the same color under almost horizontal light.

In another one of its aspects, the invention provides a cosmetic composition comprising, in a cosmetically acceptable medium:
at least one red interference pigment that, when the composition is applied to a support, can generate highlights with an intensity of 3000 cd·m⁻² or more and a dominant wavelength in the range 580 nm to 680 nm; and
at least one coloring agent which is sensitive to at least one external stimulus.

The combined use of a red interference pigment and the Xchrome coloring agent can produce at least two different appearances for the composition depending on the state of the Xchrome coloring agent.

It may be particularly esthetically pleasing if, in one of its states, the Xchrome coloring agent takes on a red color since that can reduce the contrast of the red highlights and render them less visible. The change in state of the Xchrome coloring agent is thus accompanied by better perception of the red highlights and the observer may be surprised to see the interference pigment shine intensely.

Further, by changing state, the Xchrome coloring agent may influence the diffusion of light in the environment of the red interference pigment by acting as a color filter or locally as a secondary source of illumination.

In one example of the invention, the Xchrome coloring agent may be selected so that it takes at least two states in which the interference phenomenon is and is not affected or in which it is affected to different degrees.

The coloring agent that is sensitive to an external stimulus may be in solution in the medium, which may apply with a solvatochromic agent, for example. This may avoid diffusion of light by the Xchrome agent and weaken the interference phenomenon.

It may be particularly advantageous for the red interference pigment to have a dimension in the range 30 μm [micrometer] to 80 μm, i.e. substantially of the same order as the separating power of the eye, more preferably about 40 μm, and for the Xchrome coloring agent to take on a red color in one of its states. Thus, a matte red background is obtained with highlights that appear to scintillate because of their particular dimensions, creating a sparkling effect.

In another one of its aspects, the invention provides a set comprising:
a first cosmetic composition for applying to keratinous substances, and comprising at least a diffusing filler or a coloring agent that is capable of generating a color by absorption, and
a second cosmetic composition for applying on the first and comprising a cosmetically acceptable medium in which there is dispersed at least one red interference pigment that, when the second composition is applied to a surface, is capable of creating highlights with an intensity of 3000 cd·m⁻² or more and with a dominant wavelength in the range 580 nm to 650 nm.

By means of this aspect of the invention, the interference phenomenon is not hampered by the presence of the diffusing pigment or of the filler since said pigment or said filler is present in the underlying base layer and consequently does not deleteriously affect the propagation of light in the covering layer containing the red interference pigment.

The medium in which the red interference pigment is dispersed is preferably transparent, thereby making it possible to see the underlying deposit.

In another one of its aspects, the invention also provides a set comprising:
a base composition comprising a cosmetically acceptable medium in which there is dispersed at least one red interference pigment that, when the composition is applied to a surface, is capable of creating highlights with an intensity of 3000 cd·m⁻² or more and with a dominant wavelength in the range 580 nm to 650 nm,
a covering composition for applying on the base composition. This other composition may be transparent and may serve, for example, to improve glossiness and create a magnifying-glass effect on the red highlight points.

The covering composition may comprise a medium having a refractive index that is greater than the refractive index of the medium in which the red interference pigment is dispersed.

The first composition may be for forming the base layer and may present any formulation that is compatible with subsequently depositing the second composition.

In particular, the first composition may comprise a cosmetically acceptable medium, as defined above, and at least one coloring agent or a diffusing filler.

The second composition contains the red interference pigment, dispersed in a cosmetically acceptable medium.

The second composition is for applying on the first, for example.

Measurement of the Intensity of the Overbrightness Points

To measure the intensity of the overbrightness points, the composition studied is spread onto a contrast card, for example of Leneta brand, to a thickness of 300 μm.

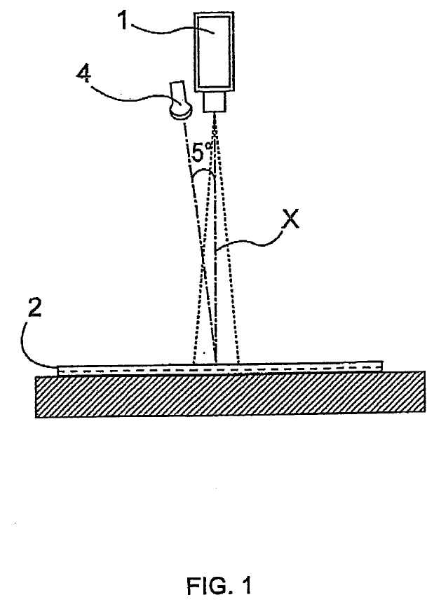

The composition thus spread out is placed in front of a calorimetric camera 1 according to the arrangement shown in FIG. 1. In this figure, it is seen that the contrast card 2 coated with the composition is placed perpendicular to the optical axis X of the camera 1 and that the lighting is provided by means of a light source 4 (illuminant D65) emitting in a direction forming an angle of 5° with the optical axis X.

Overbrightness is defined as being the light intensity emitted in a localized manner.

The camera has a resolution in the plane xy of a few μm, sufficient to very clearly differentiate the various particles present in the composition.

The optical system is, for example, the photometer and the imaging calorimeter Lumicam 1300 from the company Instrument System.

The luminance measurements may be performed in the range from 0.2 to 200 000 cd·m⁻² with a measuring accuracy of 4%, a repeatability of 0.1% and a uniformity of 1.5% (for an area of 10×10 pixels).

The optical system comprises a 105 mm macro objective lens with a field angle of 5° and a focal length of 22 mm, placed 48 cm from the composition. The measuring area extends over 2.9×2.7 mm.

The sensitivity is 100 iso, the shutter speed is 1/60 sec and the aperture is f:2.

The experimental device illustrated makes it possible to eliminate the specular reflection on the surface of the film of the composition.

The result obtained is in the form of a two-dimensional matrix in which each component M_{i,j} represents the intensity detected by the cell of coordinates i,j in the plane xy, in candelas per m²,

$$[ \begin{matrix}{M}_{1 , 1} & & … & & {M}_{1 , m} \\ & ⋰ & & ⋱ & \\ ⋮ & & {M}_{i , j} & & ⋮ \\ & ⋱ & & ⋰ & \\ {M}_{n , 1} & & … & & {M}_{m , n}\end{matrix} ]$$

in which:

m denotes the number of pixels in the x direction of the detection system, and

n denotes the number of pixels in the y direction of the detection system.

The dominant wavelength may be measured with the calorimeter.

Turbidity Measurement

Turbidity corresponds to the reduction in the transparency of a liquid as a result of the presence of particles in suspension, and is measured by passing a light beam through the sample being tested.

Turbidity can depend on the refractive index of the medium and on the kind and the concentration of bodies in suspension in said medium.

The turbidity index is determined by measuring the light that is diffused by the particles in suspension, by means of a tubidimeter, in this event the turbidimeter referenced 2100 P by HACH.

Measurement of the Color Path

When the composition presents a turbidity index of 100 nephelometric turbidity units (NTU) or less, it makes it possible to obtain a relatively long color path, since the small total amount of particles in suspension does not hamper observation of the color produced by absorption by the surface layer of the high-index red interference pigment.

The term “color path” denotes a variation in the a*b* plane of the CIE 1976 calorimetric space and can, for example, be measured by means of a spectrogonioreflectometer of trade name INSTRUMENT SYSTEMS and of reference GON 360 GONIOMETER after the composition has been spread in the fluid state to a thickness of 300 μm by means of an automatic spreader onto a contrast card of trade name ERICHSEN and of reference Typ 24/5, the measurement being taken on the black background of the card.

The color path of a composition of the invention corresponds to a variation Dh in the hue angle h of at least 20°, for example, when the observation angle is varied in the range 0 to 80° relative to the normal, for a light at an angle of incidence of 45°.

Red Interference Pigment

This pigment is capable, according to the invention, of generating overbrightness points with a dominant wavelength of between 580 nm and 650 nm and better still 580 nm and 600 nm, and with an intensity of greater than or equal to 3500 cd·m⁻² and better still 4200 cd·m⁻². The intensity may be less than 5000 cd·m⁻².

Preferably, the size of this pigment, defined by the mean particle size distribution of half the population, also known as the D₅₀, is greater than or equal to 30 μm and better still 40 μm, for example between 30 and 80 μm and better still between 30 and 70 μm.

The pigment advantageously has a flattened general shape, its thickness being, for example, less than or equal to 5 μm and preferably less than or equal to 3 μm.

The multilayer structure may be symmetrical or unsymmetrical, and is preferably symmetrical.

The pigment may comprise a core of an organic or inorganic material covered with one or more layers of organic or inorganic materials.

The pigment may comprise, for example, a silica, mica or glass core, coated with a layer of iron oxide Fe₂O₃ or of another metal oxide, for example a titanium or tin oxide.

The thickness of the various layers covering the core will be determined by the theory of light reflection on thin films, such that the reflected light has the desired dominant wavelength.

Preferably, the core is of flattened general shape and the pigment has substantially flat main faces, so as to allow strong specular reflection.

The pigment may, where appropriate, have non-zero magnetic susceptibility.

An example of a commercially available red interference pigment that may be mentioned is the product sold under the reference Xirona Red by the company Merck.

Cosmetically Acceptable Medium

The cosmetically acceptable medium will be adapted to the nature of the support onto which the composition is to be applied, and also to the form in which the composition is intended to be conditioned.

The composition according to the invention comprises an aqueous medium.

Aqueous Phase

The composition may comprise water or a mixture of water and of hydrophilic organic solvents, for instance alcohols and especially linear or branched lower monoalcohols containing from 2 to 5 carbon atoms, for instance ethanol, isopropanol or n-propanol, polyols, for instance glycerol, diglycerol, propylene glycol, sorbitol or pentylene glycol, and polyethylene glycols.

The hydrophilic phase may also contain hydrophilic C₂ ethers and C₂-C₄ aldehydes.

Water or a mixture of water and of hydrophilic organic solvents may be present in the composition according to the invention in a content ranging from 0 to 90%, especially 0.1% to 90% by weight, preferably from 0 to 60% by weight and especially 0.1% to 60% by weight, relative to the total weight of the composition.

The medium may comprise a liquid organic phase in which water is dispersed or emulsified, on condition that the red interference pigment is in major amount in the aqueous phase.

Film-Forming Agent

The medium may comprise a film-forming agent, especially a film-forming polymer, for example in a content ranging from 1% to 90% depending on the nature of the composition.

The term “film-forming agent” means an agent capable of forming, by itself or in the presence of an auxiliary film-forming agent, a macroscopically continuous film that adheres to keratin materials, and preferably a cohesive film, and better still a film whose cohesion and mechanical properties are such that the said film may be isolable and manipulable in isolation, for example when the said film is prepared by pouring onto a non-stick surface, for instance a Teflon-coated or silicone-coated surface.

The film-forming agent may or may not be present in the aqueous phase. This agent may be in dispersion or in solution in the aqueous phase, while avoiding excessively unfavourably affecting the refractive index.

The film-forming agent may be a film-forming polymer.

Film-Forming Polymer

The term “film-forming” polymer means a polymer capable, by itself or in the presence of an auxiliary film-forming agent, of forming a continuous film that adheres to a support, especially to keratin materials, preferably a cohesive film and better still a film whose cohesion and mechanical properties are such that the said film may be isolated from the said support.

Among the film-forming polymers that may be used in the composition of the present invention, mention may be made of synthetic polymers, of free-radical type or of polycondensate type, and polymers of natural origin, and mixtures thereof.

Film-forming polymers that may be mentioned in particular include acrylic polymers, polyurethanes, polyesters, polyamides, polyureas and cellulose-based polymers, for instance nitrocellulose.

These film-forming polymers may be divided into four classes, as a function of their solubility with regard to an aqueous phase or a liquid fatty phase.

In one embodiment, the film-forming polymer is at least one polymer chosen from the group comprising:
film-forming polymers that are soluble in a liquid fatty phase of the composition, in particular liposoluble polymers,
film-forming polymers that are dispersible in a liquid fatty phase of the composition, in particular polymers in the form of non-aqueous dispersions of polymer particles, preferably dispersions in silicone oils or hydrocarbon-based oils,
aqueous dispersions of film-forming polymer particles, often known as “latices”,
water-soluble film-forming polymers.

According to another embodiment of the invention, the film-forming polymer is silicone-based and may be chosen from polymers with a non-silicone organic backbone grafted with monomers containing a polysiloxane.

According to another embodiment of the invention, the film-forming polymer is silicone-based and is chosen from silicone polymers grafted with non-silicone organic monomers. These polymers may be liposoluble, lipodispersible, water-soluble or dispersible in aqueous medium, where appropriate.

For obvious reasons, the amounts of film-forming agent in the compositions according to the invention may vary significantly, especially with regard to the nature of the film-forming agent under consideration and also with regard to the qualities desired for the composition incorporating it.

The composition may comprise, as polymer, a dispersion of particles of a grafted ethylenic polymer in a liquid fatty phase.

The term “ethylenic” polymer means a polymer obtained by polymerization of ethylenically unsaturated monomers.

The dispersion of grafted ethylenic polymer is especially free of stabilizing polymer different from the said grafted polymer, such as those described in EP 749 747 and described hereinbelow, and the particles of grafted ethylenic polymer are therefore not surface-stabilized with such additional stabilizing polymers. The grafted polymer is therefore dispersed in the liquid fatty phase in the absence of additional surface stabilizer for the particles.

The term “grafted” polymer means a polymer having a backbone comprising at least one side chain that is pendent or located at the end of a chain, and preferably pendent.

Advantageously, the grafted ethylenic polymer comprises an ethylenic backbone that is insoluble in the said liquid fatty phase, and side chains covalently bonded to the said backbone, which are soluble in the liquid fatty phase.

The grafted ethylenic polymer is especially a non-crosslinked polymer. In particular, the polymer is obtained by polymerization of monomers comprising only one polymerizable group.

The grafted ethylenic polymer is, for example, a grafted acrylic polymer.

The grafted ethylenic polymer may especially be obtained by free-radical polymerization in an organic polymerization medium:
of at least one ethylenic monomer, in particular of at least one acrylic monomer and optionally of at least one additional non-acrylic vinyl monomer, to form the said insoluble backbone; and
of at least one macromonomer comprising a polymerizable end group to form the side chains, the said macromonomer having a weight-average molar mass of greater than or equal to 200 and the content of polymerized macromonomer representing from 0.05% to 20% by weight of the polymer.

The composition may comprise a liquid fatty phase that may contain the organic polymerization medium for the grafted ethylenic polymer.

The organic liquid dispersion medium, corresponding to the medium in which the grafted polymer is supplied, may be identical to the polymerization medium.

However, the polymerization medium may be totally or partially replaced with another organic liquid medium. This other organic liquid medium may be added, after polymerization, to the polymerization medium. The said polymerization medium is then totally or partially evaporated.

The liquid fatty phase may contain liquid organic compounds other than those present in the dispersion medium. These other compounds are chosen such that the grafted polymer remains in dispersed form in the liquid fatty phase.

The organic liquid dispersion medium may be present in a liquid fatty phase of the composition according to the invention due to the introduction into the composition of the dispersion of grafted polymer obtained.

Such a liquid fatty phase may comprise, preferably predominantly, one or more liquid organic compounds (or oils) as defined below.

In particular, the composition may comprise a liquid fatty phase that may be a non-aqueous liquid organic phase that is immiscible with water at room temperature (25° C.).

The term “liquid organic compound” means a non-aqueous compound that is in liquid form at room temperature (25° C.) and therefore flows under its own weight.

Among the liquid organic compounds or oils that may be present in the liquid organic dispersion medium, mention may be made of:
liquid organic compounds, especially silicone-based or non-silicone-based, having a global solubility parameter according to the Hansen solubility space of less than or equal to 18 (MPa)^{1/2} and preferably less than or equal to 17 (MPa)^{1/2},
monoalcohols having a global solubility parameter according to the Hansen solubility space of less than or equal to 20 (MPa)^{1/2}, and
mixtures thereof.

The global solubility parameter δ according to the Hansen solubility space is defined in the article “Solubility parameter values” by Eric A. Grulke in the book “Polymer Handbook”, 3rd Edition, Chapter VII, p. 519-559, by the relationship:
δ=(δ_{D}²+δ_{P}²+δ_{H}²)^{1/2}

in which
δ_{D} characterizes the London dispersion forces arising from the formation of dipoles induced during molecular impacts,
δ_{P} characterizes the Debye interaction forces between permanent dipoles, and
δ_{H} characterizes the forces of specific interactions (such as hydrogen bonding, acid/base, donor/acceptor, etc.).

The definition of solvents in the solubility space according to Hansen is described in the article by C. M. Hansen: “The three-dimensional solubility parameters”, J. Paint Technol. 39, 105 (1967).

Among the liquid organic compounds, especially silicone-based or non-silicone-based, having a global solubility parameter according to the Hansen solubility space of less than or equal to 18 (MPa)^{1/2}, mention may be made of liquid fatty substances, especially oils, which may be chosen from natural or synthetic, carbon-based, hydrocarbon-based, fluoro and silicone oils, which are optionally branched, alone or as a mixture.

Among these oils, mention may be made of plant oils formed from fatty acid esters and from polyols, in particular triglycerides, such as sunflower oil, sesame oil or rapeseed oil, or esters derived from acids or alcohols containing a long chain (i.e. a chain containing from 6 to 20 carbon atoms), in particular the esters of formula RCOOR′ in which R represents a higher fatty acid residue containing from 7 to 19 carbon atoms and R′ represents a hydrocarbon-based chain containing from 3 to 20 carbon atoms, such as palmitates, adipates and benzoates, in particular diisopropyl adipate.

Mention may also be made of linear, branched and/or cyclic alkanes that may be volatile, and in particular liquid paraffin, liquid petroleum jelly or hydrogenated polyisobutylene, isododecane or “Isopars”, volatile isoparaffins. Mention may also be made of esters, ethers and ketones.

Mention may also be made of silicone oils such as polydimethylsiloxanes and polymethylphenylsiloxanes, optionally substituted with aliphatic and/or aromatic groups, which are optionally fluorinated, or with functional groups such as hydroxyl, thiol and/or amine groups, and volatile silicone oils, which are especially cyclic.

In particular, mention may be made of volatile and/or non-volatile, optionally branched silicone oils.

As non-silicone-based liquid organic compounds with a global solubility parameter according to the Hansen solubility space of less than or equal to 18 (MPa)^{1/2}, mention may be made in particular of:
linear, branched or cyclic esters containing at least 6 carbon atoms, especially 6 to 30 carbon atoms;
ethers containing at least 6 carbon atoms, especially 6 to 30 carbon atoms; and
ketones containing at least 6 carbon atoms, especially 6 to 30 carbon atoms.

The expression “liquid monoalcohols having a global solubility parameter according to the Hansen solubility space of less than or equal to 20 (MPa)^{1/2}”, means aliphatic fatty liquid monoalcohols containing from 6 to 30 carbon atoms, the hydrocarbon-based chain not comprising a substitution group. Monoalcohols according to the invention that may be mentioned include oleyl alcohol, decanol, octyldodecanol and linoleyl alcohol.

When the composition comprises a non-silicone liquid fatty phase, the macromonomers present in the grafted polymer are advantageously carbon-based macromonomers as described below.

In particular, when the composition comprises a non-silicone liquid fatty phase, the grafted polymer present in the composition is advantageously a non-silicone grafted polymer.

The term “non-silicone grafted polymer” means a grafted polymer mainly containing a carbon-based macromonomer and optionally containing not more than 7% by weight and preferably not more than 5% by weight of silicone macromonomer, or even being free of silicone macromonomer.

When the cosmetic composition according to the invention comprises a silicone-based liquid fatty phase, the macromonomers present in the grafted polymer are advantageously silicone-based macromonomers as described below.

In particular, when the liquid fatty phase is a silicone-based liquid fatty phase, the grafted polymer present in the composition is advantageously a silicone-based grafted polymer.

The term “silicone-based grafted polymer” means a grafted polymer predominantly containing a silicone-based macromonomer and optionally containing up to 7% by weight and preferably up to 5% by weight of carbon-based macromonomer, or even being free of carbon-based macromonomer.

a) Monomers

The choice of monomers constituting the backbone of the polymer, of macromonomers, the molecular weight of the polymer, and the proportion of the monomers and macromonomers may be made as a function of the liquid organic dispersion medium so as advantageously to obtain a dispersion of particles of grafted polymers, in particular a stable dispersion, this choice possibly being made by a person skilled in the art.

The term “stable dispersion” means a dispersion that is not liable to form a solid deposit or to undergo liquid/solid phase separation, especially after centrifugation, for example at 4000 rpm for 15 minutes.

The grafted ethylenic polymer forming the particles in dispersion thus comprises a backbone that is insoluble in the said dispersion medium and a portion that is soluble in the said dispersion medium.

The grafted ethylenic polymer may be a random polymer.

According to the invention, the term “grafted ethylenic polymer” means a polymer that may be obtained by free-radical polymerization:
of one or more ethylenic monomer(s);
with one or more macromonomer(s), in an organic polymerization medium.

According to the invention, the term “grafted acrylic polymer” means a polymer that may be obtained by free-radical polymerization:
of one or more acrylic monomer(s), and optionally of one or more additional non-acrylic vinyl monomer(s);
with one or more macromonomer(s), in an organic polymerization medium.

Advantageously, the acrylic monomers represent from 50% to 100% by weight, preferably from 55% to 100% by weight (especially from 55% to 95% by weight) and preferentially from 60% to 100% by weight (especially from 60% to 90% by weight) of the mixture of acrylic monomers+optional non-acrylic vinyl monomers.

In particular, the acrylic monomers are chosen from monomers whose homopolymer is insoluble in the dispersion medium under consideration, i.e. the homopolymer is in solid (or non-dissolved) form at a concentration of greater than or equal to 5% by weight at room temperature (20° C.) in the said dispersion medium.

According to the invention, the expression “macromonomer containing a polymerizable end group” means any polymer comprising on only one of its ends a polymerizable end group capable of reacting during the polymerization reaction with acrylic monomers and optionally the additional non-acrylic vinyl monomers constituting the backbone. The macromonomer makes it possible to form the side chains of the grafted acrylic polymer. The polymerizable group of the macromonomer may advantageously be an ethylenically unsaturated group capable of free-radical polymerization with the monomers constituting the backbone.

The term “carbon-based macromonomer” means a non-silicone-based macromonomer and especially an oligomeric macromonomer obtained by polymerization of ethylenically unsaturated non-silicone-based monomer(s), and mainly by polymerization of acrylic and/or non-acrylic vinyl monomers.

The term “silicone-based macromonomer” means an organopolysiloxane macromonomer and in particular a polydimethylsiloxane macromonomer.

In particular, the macromonomer is chosen from macromonomers whose homopolymer is soluble in the dispersion medium under consideration, i.e. fully dissolved at a concentration of greater than or equal to 5% by weight and at room temperature in the said dispersion medium.

Thus, the grafted acrylic polymer comprises a backbone (or main chain) consisting of a sequence of acrylic units resulting from the polymerization especially of one or more acrylic monomers and of side chains (or grafts) derived from the reaction of the macromonomers, the said side chains being covalently bonded to the said main chain.

The backbone (or main chain) is insoluble in the dispersion medium under consideration, whereas the side chains (or grafts) are soluble in the said dispersion medium.

In the present patent application, the term “acrylic monomers” means monomers chosen from (meth)acrylic acid, (meth)acrylic acid esters (also known as (meth)acrylates), and (meth)acrylic acid amides (also known as (meth)acrylamides).

As acrylic monomers that may be used to constitute the insoluble backbone of the polymer, mention may be made, alone or as a mixture, of the following monomers, and also the salts thereof:
(i) the (meth)acrylates of formula (VIII): 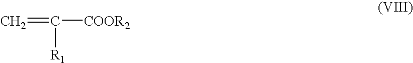
   in which:
R₁ denotes a hydrogen atom or a methyl group;
R₂ represents a group chosen from:
   a linear or branched alkyl group containing from 1 to 6 carbon atoms, the said group possibly comprising in its chain one or more hetero atoms chosen from O, N and S; and/or possibly comprising one or more substituents chosen from —OH, halogen atoms (F, Cl, Br or I) and —NR′R″ with R′ and R″, which may be identical or different, chosen from linear or branched C₁-C₄ alkyls; and/or possibly being substituted with at least one polyoxyalkylene group, in particular with C₂-C₄ alkylene, especially polyoxyethylene and/or polyoxypropylene, the said polyoxyalkylene group consisting of a repetition of 5 to 30 oxyalkylene units;
   a cyclic alkyl group containing from 3 to 6 carbon atoms, the said group possibly comprising in its chain one or more hetero atoms chosen from O, N and S, and/or possibly comprising one or more substituents chosen from OH and halogen atoms (F, Cl, Br or I).

Examples of R₂ that may be mentioned include the methyl, ethyl, propyl, butyl, isobutyl, methoxyethyl, ethoxyethyl, methoxypolyoxyethylene (350 OE), trifluoroethyl, 2-hydroxyethyl, 2-hydroxypropyl, dimethylaminoethyl, diethylaminoethyl or dimethylaminopropyl group;
(ii) the (meth)acrylamides of formula (IX): 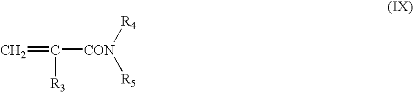
   in which:
R₃ denotes a hydrogen atom or a methyl group;
R₄ and R₅, which may be identical or different, represent a hydrogen atom or a linear or branched alkyl group containing from 1 to 6 carbon atoms, which may comprise one or more substituents chosen from —OH, halogen atoms (F, Cl, Br or I) and —NR′R″ with R′ and R″, which may be identical or different, chosen from linear or branched C₁-C₄ alkyls; or
R₄ represents a hydrogen atom and R₅ represents a 1,1-dimethyl-3-oxobutyl group.

As examples of alkyl groups that can constitute R₄ and R₅, mention may be made of n-butyl, t-butyl, n-propyl, dimethylaminoethyl, diethylaminoethyl and dimethylaminopropyl;
(iii) (meth)acrylic monomers comprising at least one carboxylic acid, phosphoric acid or sulfonic acid function, such as acrylic acid, methacrylic acid or acrylamidopropanesulfonic acid.

Among these acrylic monomers, those that may be mentioned most particularly are methyl, ethyl, propyl, butyl and isobutyl (meth)acrylates; methoxyethyl or ethoxyethyl (meth)acrylates; trifluoroethyl methacrylate; dimethylaminoethyl methacrylate, diethylaminoethyl methacrylate, 2-hydroxypropyl methacrylate, 2-hydroxyethyl methacrylate, 2-hydroxypropyl acrylate, 2-hydroxyethyl acrylate; dimethylaminopropylmethacrylamide; and the salts thereof; and mixtures thereof.

In particular, the acrylic monomers are chosen from methyl acrylate, methoxyethyl acrylate, methyl methacrylate, 2-hydroxyethyl methacrylate, acrylic acid and dimethylaminoethyl methacrylate, and mixtures thereof.

Among the additional non-acrylic vinyl monomers that may be mentioned are:
vinyl esters of the following formula:
   R₆—COO—CH═CH₂
   in which:
R₆ represents a linear or branched alkyl group containing from 1 to 6 atoms, or a cyclic alkyl group containing from 3 to 6 carbon atoms and/or an aromatic group, for example of benzene, anthracene or naphthalene type;
non-acrylic vinyl monomers comprising at least one carboxylic acid, phosphoric acid or sulfonic acid function, such as crotonic acid, maleic anhydride, itaconic acid, fumaric acid, maleic acid, styrenesulfonic acid, vinylbenzoic acid or vinylphosphoric acid, and the salts thereof;
non-acrylic vinyl monomers comprising at least one tertiary amine function, such as 2-vinylpyridine or 4-vinylpyridine;
and mixtures thereof.

Advantageously, the acrylic monomers present in the grafted polymer comprise at least (meth)acrylic acid and at least one monomer chosen from the (meth)acrylates and (meth)acrylamides described previously in points (i) and (ii). Preferably, the acrylic monomers comprise at least (meth)acrylic acid and at least one monomer chosen from C₁-C₃ alkyl (meth)acrylates. (Meth)acrylic acid may be present in a content of at least 5% by weight, especially ranging from 5% to 80% by weight, preferably of at least 10% by weight, especially ranging from 10% to 70% by weight, and preferentially of at least 15% by weight, especially ranging from 15% to 60% by weight, relative to the total weight of the polymer.

Among the salts that may be mentioned are those obtained by neutralization of acid groups with mineral bases such as sodium hydroxide, potassium hydroxide or ammonium hydroxide, or organic bases such as alkanolamines, for instance monoethanolamine, diethanolamine, triethanolamine or 2-methyl-2-amino-1-propanol.

Mention may also be made of the salts formed by neutralization of tertiary amine units, for example using a mineral or organic acid. Among the mineral acids that may be mentioned are sulfuric acid, hydrochloric acid, hydrobromic acid, hydriodic acid, phosphoric acid and boric acid. Among the organic acids that may be mentioned are acids comprising one or more carboxylic, sulfonic or phosphonic groups. They may be linear, branched or cyclic aliphatic acids, or alternatively aromatic acids. These acids may also comprise one or more hetero atoms chosen from O and N, for example in the form of hydroxyl groups. Acetic acid or propionic acid, terephthalic acid, and citric acid and tartaric acid may especially be mentioned.

According to one embodiment of the invention, the grafted ethylenic polymer contains no additional non-acrylic vinyl monomers as described above. In this embodiment, the insoluble backbone of the grafted ethylenic polymer is formed solely from acrylic monomers as described previously.

It is understood that these non-polymerized acrylic monomers may be soluble in the dispersion medium under consideration, but the polymer formed with these monomers is insoluble in the dispersion medium.

According to one particular embodiment of the invention, the grafted ethylenic polymer may be obtained by free-radical polymerization in an organic polymerization medium:
of a main acrylic monomer chosen from C₁-C₃ alkyl (meth)acrylates, alone or as a mixture, and optionally of one or more additional acrylic monomers chosen from (meth)acrylic acid, methacrylic acid and alkyl(meth)acrylates of formula (X) defined below, and salts thereof, to form the said insoluble backbone; and
of at least one silicone-based macromonomer comprising a polymerizable end group, as defined previously.

Main acrylic monomers that may be used include methyl acrylate, methyl methacrylate, ethyl acrylate, ethyl methacrylate, n-propyl acrylate, n-propyl methacrylate, isopropyl acrylate and isopropyl methacrylate, and mixtures thereof.

Methyl acrylate, methyl methacrylate and ethyl methacrylate may be mentioned most particularly.

The additional acrylic monomers may be chosen from:
(meth)acrylic acid and its salts,
the (meth)acrylates of formula (X), and salts thereof: 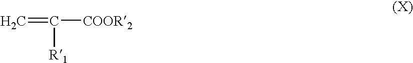
   in which:
R′₁ denotes a hydrogen atom or a methyl group;
R′₂ represents
   a linear or branched alkyl group containing from 1 to 6 carbon atoms, the said group comprising in its chain one or more oxygen atoms and/or comprising one or more substituents chosen from —OH, halogen atoms (F, Cl, Br or I) and —NR′R″, with R′ and R″, which may be identical or different, being chosen from linear or branched C₁-C₃ alkyls;
a cyclic alkyl group containing from 3 to 6 carbon atoms, the said group possibly comprising in its chain one or more oxygen atoms and/or possibly comprising one or more substituents chosen from OH and halogen atoms (F, Cl, Br or I);
and mixtures thereof.

Examples of R′₂ that may be mentioned include the methoxyethyl, ethoxyethyl, trifluoroethyl; 2-hydroxyethyl, 2-hydroxypropyl, dimethylaminoethyl, diethylaminoethyl and dimethylaminopropyl groups.

Among these additional acrylic monomers, mention may be made most particularly of (meth)acrylic acid, methoxyethyl or ethoxyethyl (meth)acrylates; trifluoroethyl methacrylate; dimethylaminoethyl methacrylate, diethylaminoethyl methacrylate, 2-hydroxypropyl methacrylate, 2-hydroxyethyl methacrylate, 2-hydroxypropyl acrylate and 2-hydroxyethyl acrylate, the salts thereof, and mixtures thereof.

Acrylic acid and methacrylic acid may be mentioned most particularly.

b) Macromonomers

The macromonomers comprise at one of the ends of the chain a polymerizable end group capable of reacting during the polymerization with the acrylic monomers and optionally the additional vinyl monomers, to form the side chains of the grafted ethylenic polymer. The said polymerizable end group may in particular be a vinyl or (meth)acrylate (or (meth)-acryloxy) group, and preferably a (meth)acrylate group.

The macromonomers are preferably chosen from macromonomers whose homopolymer has a glass transition temperature (Tg) of less than or equal to 25° C., especially ranging from −100° C. to 25° C. and preferably ranging from −80° C. to 0° C.

The macromonomers have a weight-average molar mass of greater than or equal to 200, preferably greater than or equal to 300, preferentially greater than or equal to 500 and more preferentially greater than 600.

Preferably, the macromonomers have a weight-average molar mass (Mw) ranging from 200 to 100 000, preferably ranging from 500 to 50 000, preferentially ranging from 800 to 20 000, more preferentially ranging from 800 to 10 000 and even more preferentially ranging from 800 to 6000.

In the present patent application, the weight-average (Mw) and number-average (Mn) molar masses are determined by liquid gel permeation chromatography (THF solvent, calibration curve established with linear polystyrene standards, refractometric detector).

Carbon-based macromonomers that may in particular be mentioned include:
(i) homopolymers and copolymers of linear or branched C₈-C₂₂ alkyl acrylate or methacrylate, containing a polymerizable end group chosen from vinyl or (meth)acrylate groups, among which mention may be made in particular of: poly(2-ethylhexyl acrylate) macromonomers with a mono(meth)acrylate end group; poly(dodecyl acrylate) or poly(dodecyl methacrylate) macromonomers with a mono(meth)acrylate end group; poly(stearyl acrylate) or poly(stearyl methacrylate) macromonomers with a mono(meth)acrylate end group.

Such macromonomers are described in particular in the patents EP 895 467 and EP 96459, and in the article by Gillman K. F., Polymer Letters, Vol 5, page 477-481 (1967).

Mention may be made in particular of macromonomers based on poly(2-ethylhexyl acrylate) or poly(dodecyl acrylate) with a mono(meth)acrylate end group;
(ii) polyolefins containing an ethylenically unsaturated end group, in particular containing a (meth)acrylate end group. Examples of such polyolefins that may be mentioned in particular include the following macromonomers, it being understood that they have a (meth)acrylate end group: polyethylene macromonomers, polypropylene macromonomers, macromonomers of polyethylene/polypropylene copolymer, macromonomers of polyethylene/polybutylene copolymer, polyisobutylene macromonomers; polybutadiene macromonomers; polyisoprene macromonomers; polybutadiene macromonomers; poly(ethylene/butylene)-polyisoprene macromonomers.

Such macromonomers are described in particular in U.S. Pat. No. 5,625,005, which mentions ethylene/butylene and ethylene/propylene macromonomers containing a (meth)acrylate reactive end group.

Mention may be made in particular of the poly(ethylene/butylene) methacrylate such as that sold under the name Kraton Liquid L-1253 by Kraton Polymers.

Silicone-based macromonomers that may be mentioned in particular include polydimethylsiloxanes containing mono(meth)acrylate end groups, and especially those of formula (XI) below: 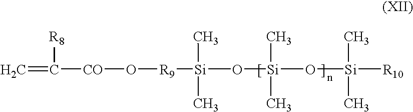
in which:
R₈ denotes a hydrogen atom or a methyl group;
R₉ denotes a divalent hydrocarbon-based group containing from 1 to 10 carbon atoms and optionally contains one or two ether bonds —O—;
R₁₀ denotes an alkyl group containing from 1 to 10 carbon atoms and especially from 2 to 8 carbon atoms; and
n denotes an integer ranging from 1 to 300, preferably ranging from 3 to 200 and preferentially ranging from 5 to 100.

Silicone-based macromonomers that may be used include monomethacryloxypropyl polydimethylsiloxanes such as those sold under the name PS560-K6 by the company United Chemical Technologies Inc. (UCT) or under the name MCR-M17 by the company Gelest Inc.

More particularly, the polymerized macromonomer (constituting the side chains of the grafted polymer) represents from 0.1% to 15% by weight, preferably from 0.2% to 10% by weight and more preferably from 0.3% to 8% by weight, relative to the total weight of the polymer.

As particularly preferred grafted ethylenic polymer dispersed in a non-silicone-based liquid fatty phase, it is possible to use those obtained by polymerization:
of methyl acrylate and of a polyethylene/polybutylene macromonomer containing a methacrylate end group (especially Kraton L-1253), in particular in a solvent chosen from isododecane, isononyl isononanoate, octyldodecanol, diisostearyl malate or a C₁₂-C₁₅ alkyl benzoate (such as Finsolv Tenn.);
of methoxyethyl acrylate and of a polyethylene/polybutylene macromonomer containing a methacrylate end group (especially Kraton L-1253), in particular in isododecane;
of methyl acrylate/methyl methacrylate monomers and of a polyethylene/polybutylene macromonomer containing a methacrylate end group (especially Kraton L-1253), in particular in isododecane;
of methyl acrylate/acrylic acid monomers and of a polyethylene/polybutylene macromonomer containing a methacrylate end group (especially Kraton L-1253), in particular in isododecane;
of methyl acrylate/dimethylaminoethyl methacrylate monomers and of a polyethylene/polybutylene macromonomer containing a methacrylate end group (especially Kraton L-1253), in particular in isododecane;
of methyl acrylate/2-hydroxyethyl methacrylate monomers and of a polyethylene/polybutylene macromonomer containing a methacrylate end group (especially Kraton L-1253), in particular in isododecane.

As particularly envisaged grafted acrylic polymer dispersed in a silicone-based liquid fatty phase, it is possible to use those obtained by polymerization:
of methyl acrylate and of the monomethacryloyl-oxypropyl polydimethylsiloxane macromonomer with a weight-average molecular weight ranging from 800 to 6000, in particular in decamethylcyclopentasiloxane or phenyl trimethicone;
of methyl acrylate, acrylic acid and the monometh-acryloxypropyl polydimethylsiloxane macromonomer with a weight-average molecular weight ranging from 800 to 6000, in particular in decamethylcyclopentasiloxane or phenyl trimethicone.

In particular, the grafted polymer has a weight-average molar mass (Mw) of between 10 000 and 300 000, especially between 20 000 and 200 000 and better still between 25 000 and 150 000.

By virtue of the abovementioned characteristics, in a given organic dispersion medium, the polymers have the capacity of folding over on themselves, thus forming particles of substantially spherical shape, the periphery of these particles having the deployed side chains, which ensure the stability of these particles. Such particles resulting from the characteristics of the grafted polymer have the particular feature of not aggregating in the said medium and thus of being self-stabilized and of forming a particularly stable polymer particle dispersion.

In particular, the grafted ethylenic polymers of the dispersion are capable of forming nanometre-sized particles, with a mean size ranging from 10 to 400 nm and preferably from 20 to 200 nm.

As a result of this very small size, the grafted polymer particles in dispersion are particularly stable and therefore have little susceptibility to form aggregates.

The dispersion of grafted polymer may thus be a dispersion that is stable and does not form sediments when it is placed at room temperature (25° C.) for an extended period (for example 24 hours).

In particular, the dispersion of grafted polymer particles has a solids content (or dry extract) of polymer of from 40% to 70% by weight of solids and especially from 45% to 65% by weight.

c) Production Process

The dispersion of grafted polymer particles may be prepared via a process comprising a free-radical copolymerization step, in an organic polymerization medium, of one or more acrylic monomers as defined above with one or more macromonomers as defined above.

As mentioned previously, the liquid organic dispersion medium may be identical to or different from the polymerization medium.

The copolymerization may be performed conventionally in the presence of a polymerization initiator. The polymerization initiators may be free-radical initiators. In general, such a polymerization initiator may be chosen from organic peroxide compounds such as dilauroyl peroxide, dibenzoyl peroxide or tert-butyl peroxy-2-ethylhexanoate; diazo compounds such as azobisisobutyronitrile or azobisdimethylvaleronitrile.

The reaction may also be initiated using photoinitiators or with radiation such as UV or neutrons, or with plasma.

In general, to perform this process, at least a portion of the organic polymerization medium, a portion of the additional acrylic and/or vinyl monomers, which will constitute the insoluble backbone after polymerization, all of the macromonomer (which will constitute the side chains of the polymer) and a portion of the polymerization initiator are introduced into a reactor whose size is suitable for the amount of polymer to be prepared. At this stage of introduction, the reaction medium forms a relatively homogeneous medium.

The reaction medium is then stirred and heated up to a temperature to obtain polymerization of the monomers and macromonomers. After a certain time, the initially homogeneous and clear medium leads to a dispersion of milky appearance. A mixture consisting of the remaining portion of monomers and of polymerization initiator is then added. After an adequate time during which the mixture is heated with stirring, the medium stabilizes in the form of a milky dispersion, the dispersion comprising polymer particles stabilized in the medium in which they have been created, the said stabilization being due to the presence, in the polymer, of side chains that are soluble in the said dispersion medium.

The grafted polymer may be present in the composition according to the invention in a solids content (or active material content) ranging from 1% to 70% by weight, better still from 5% to 60% by weight, preferably ranging from 6% to 45% by weight and better still ranging from 8% to 40% by weight, relative to the total weight of the composition.

In one embodiment, the film-forming polymer is an organic film-forming polymer that is soluble in a liquid fatty phase of the composition, especially in one or more oils of the composition.

In this case, it is referred to as a liposoluble polymer. The liposoluble polymer may be of any chemical type and may especially be chosen from:

a) liposoluble, amorphous homopolymers and copolymers of olefins, of cycloolefins, of butadiene, of isoprene, of styrene, of vinyl ethers, esters or amides, or of (meth)acrylic acid esters or amides comprising a linear, branched or cyclic C₄₋₅₀ alkyl group and which are preferably amorphous. The preferred liposoluble homopolymers and copolymers are obtained from monomers chosen from the group consisting of isooctyl (meth)acrylate, isononyl (meth)acrylate, 2-ethylhexyl (meth)acrylate, lauryl (meth)acrylate, isopentyl (meth)acrylate, n-butyl (meth)acrylate, isobutyl (meth)acrylate, methyl (meth)acrylate, tert-butyl (meth)acrylate, tridecyl (meth)acrylate and stearyl (meth)acrylate, or mixtures thereof. Examples that will be mentioned include the alkyl acrylate/cycloalkyl acrylate copolymer sold by Phoenix Chem. under the name Giovarez AC-5099 mL, and vinylpyrrolidone copolymers, such as copolymers of a C₂-C₃₀ and in particular C₃ to C₂₂ alkene, and combinations thereof, may be used. As examples of VP copolymers that may be used in the invention, mention may be made of copolymers of VP/vinyl laurate, VP/vinyl stearate, butylated polyvinylpyrrolidone (PVP), VP/hexadecene, VP/triacontene or VP/acrylic acid/lauryl methacrylate.

Particular liposoluble copolymers that may be mentioned include:

i) acrylic-silicone grafted polymers containing a silicone backbone and acrylic grafts or containing an acrylic backbone and silicone grafts, such as the product sold under the name SA 70.5 by 3M and described in U.S. Pat. No. 5,725,882, U.S. Pat. No. 5,209,924, U.S. Pat. No. 4,972,037, U.S. Pat. No. 4,981,903, U.S. Pat. No. 4,981,902 and U.S. Pat. No. 5,468,477, and in U.S. Pat. No. 5,219,560 and EP 0 388 582;

ii) liposoluble polymers belonging to one of the classes described above and bearing fluoro groups, in particular those described in patent U.S. Pat. No. 5,948,393 and the alkyl (meth)acrylate/perfluoroalkyl (meth)acrylate copolymers described in patents EP 0 815 836 and U.S. Pat. No. 5,849,318;

iii) polymers or copolymers resulting from the polymerization or copolymerization of an ethylenic monomer, comprising one or more ethylenic bonds, which are preferably conjugated (or diene). As polymers or copolymers resulting from the polymerization or copolymerization of an ethylenic monomer, it is possible to use vinyl, acrylic or methacrylic copolymers.

In one embodiment, the film-forming polymer is a block copolymer comprising at least one block consisting of styrene units or styrene derivatives (for example methylstyrene, chlorostyrene or chloromethylstyrene). The copolymer comprising at least one styrene block may be a diblock or triblock copolymer, or even a multiblock copolymer, in starburst or radial form. The copolymer comprising at least one styrene block may also comprise, for example, an alkylstyrene (AS) block, an ethylene/butylene (EB) block, an ethylene/propylene (EP) block, a butadiene (B) block, an isoprene (I) block, an acrylate (A) block, a methacrylate (MA) block or a combination of these blocks. The copolymer comprising at least one block consisting of styrene units or styrene derivatives may be a diblock or triblock copolymer, and in particular of the polystyrene/polyisoprene or polystyrene/polybutadiene type, such as those sold or manufactured under the name “Luvitol HSB” by BASF, and those of the polystyrene/copoly(ethylene-propylene) type or alternatively of the polystyrene/copoly(ethylene-butylene) type, such as those sold or manufactured under the brand name “Kraton” by Shell Chemical Co. or Gelled Permethyl 99A by Penreco may be used.

Examples that may be mentioned include Kraton G1650 (SEBS), Kraton G1651 (SEBS), Kraton G1652 (SEBS), Kraton G1657X (SEBS), Kraton G1701X (SEP), Kraton G1702X (SEP), Kraton G1726X (SEB), Kraton D-1101 (SBS), Kraton D-1102 (SBS), Kraton D-1107 (SIS), Gelled Permethyl 99A-750, Gelled Permethyl 99A-753-58 (blend of triblock and of starburst block polymer), Gelled Permethyl 99A-753-59 (blend of triblock and of starburst block polymer), Versagel 5970 and Versagel 5960 from Penreco (blend of triblock and of starburst polymer in isododecane).

Styrene-methacrylate copolymers may also be used, such as the polymers sold under the references OS 129880, OS 129881 and OS 84383 from Lubrizol (styrene-methacrylate copolymer).

In one embodiment, the film-forming polymer is chosen from copolymers of a vinyl ester (the vinyl group being directly attached to the oxygen atom of the ester group and the vinyl ester having a saturated, linear or branched hydrocarbon-based radical of 1 to 19 carbon atoms, linked to the carbonyl of the ester group) and of at least one other monomer, which may be a vinyl ester (other than the vinyl ester already present), an α-olefin (containing from 8 to 28 carbon atoms), an alkyl vinyl ether (the alkyl group of which contains from 2 to 18 carbon atoms) or an allylic or methallylic ester (containing a saturated, linear or branched hydrocarbon-based radical of 1 to 19 carbon atoms, linked to the carbonyl of the ester group).

These copolymers may be partially crosslinked using crosslinking agents, which may be either of the vinyl type or of the allylic or methallylic type, such as tetraallyloxyethane, divinylbenzene, divinyl octanedioate, divinyl dodecanedioate, and divinyl octadecanedioate.

Examples of these copolymers that may be mentioned include the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate-/vinyl stearate, vinyl propionate/vinyl stearate, crosslinked with 0.2% divinylbenzene, vinyl dimethylpropionate/vinyl laurate, crosslinked with 0.2% divinylbenzene, vinyl acetate/octadecyl vinyl ether, crosslinked with 0.2% tetraallyloxyethane, vinyl acetate/allyl stearate, crosslinked with 0.2% divinylbenzene, vinyl acetate/1-octadecene crosslinked with 0.2% divinylbenzene, and allyl propionate/allyl stearate, crosslinked with 0.2% divinylbenzene.

Liposoluble film-forming polymers that may also be mentioned include liposoluble copolymers, and in particular those resulting from the copolymerization of vinyl esters containing from 9 to 22 carbon atoms or of alkyl acrylates or methacrylates, the alkyl radicals containing from 10 to 20 carbon atoms.

Such liposoluble copolymers may be chosen from copolymers of polyvinyl stearate, polyvinyl stearate crosslinked with divinylbenzene, with diallyl ether or with diallyl phthalate, polystearyl (meth)acrylate copolymers, polyvinyl laurate and polylauryl (meth)acrylate, these poly(meth)acrylates possibly being crosslinked with ethylene glycol dimethacrylate or tetraethylene glycol dimethacrylate.

The liposoluble copolymers defined above are known and described especially in patent application FR-A-2 232 303; they may have a weight-average molecular weight ranging from 2000 to 500 000 and preferably from 4000 to 200 000.

As examples of liposoluble polymers that may be used in the invention, mention may be made of polyalkylenes and C₂-C₂₀ alkene copolymers, in particular polybutene.

b) amorphous and liposoluble polycondensates, in particular not comprising any groups donating hydrogen interactions, in particular aliphatic polyesters containing C₄₋₅₀ alkyl side chains or polyesters resulting from the condensation of fatty acid dimers, or even polyesters comprising a silicone-based segment in the form of a block, graft or end group, as defined in patent application FR 0 113 920, and

c) amorphous and liposoluble polysaccharides comprising alkyl (ether or ester) side chains, in particular alkylcelluloses containing a saturated or unsaturated, linear or branched C₁ to C₈ alkyl radical, such as ethylcellulose and propylcellulose.

The film-forming polymer may be chosen in particular from cellulose-based polymers such as nitrocellulose, cellulose acetate, cellulose acetobutyrate, cellulose acetopropionate or ethylcellulose, or from polyurethanes, acrylic polymers, vinyl polymers, polyvinyl butyrals, alkyd resins, resins derived from aldehyde condensation products, such as arylsulfonamide-formaldehyde resins, for instance toluenesulfonamide-formaldehyde resin, and arylsulfonamide epoxy resins.

Film-forming polymers that may especially be used include nitrocellulose RS ⅛ sec.; RS ¼ sec.; ½ sec.; RS 5 sec.; RS 15 sec.; RS 35 sec.; RS 75 sec.; RS 150 sec.; AS ¼ sec.; AS ½ sec.; SS ¼ sec.; SS ½ sec.; SS 5 sec., sold especially by the company Hercules; the toluenesulfonamide-formaldehyde resins “Ketjentflex MS80” from the company Akzo or “Santolite MHP” and “Santolite MS80” from the company Faconnier or “Resimpol 80” from the company Pan Americana, the alkyd resin “Beckosol Ode 230-70-E” from the company Dainippon, the acrylic resin “Acryloid B66” from the company Rohm & Haas, and the polyurethane resin “Trixene PR 4127” from the company Baxenden.

d) silicone resins, which are generally soluble or swellable in silicone oils. These resins are crosslinked polyorganosiloxane polymers.

The term “resin” means a three-dimensional structure.

In one embodiment, the silicone resin is chosen from silsesquioxanes and siloxysilicates.

In one embodiment, the silicone resin is chosen from siloxysilicates, such as trimethyl siloxysilicates, which are represented by the following formula:
[R₃SiO_{1/2}]ₓ—(SiO_{4/2})_{y} (units M and Q),

in which x and y may have values ranging from 50 to 80, and R represents an alkyl, such as a methyl or an alkyl of two or more carbon atoms.

The ratio of the units M to the units Q may be, for example, about 0.7:1. The film-forming silicone resin may be chosen, for example, from the resins Wacker 803 and 804, available from Wacker Silicone Corporation, and GE 1170-002 available from General Electric.

In another embodiment, the silicone resin is chosen from silsesquioxanes comprising units T:
[RSiO_{3/2}]ₜ (units T),

in which t has a value that may range up to several thousand and R represents an alkyl, such as a methyl or an alkyl of two or more carbon atoms. In one embodiment, the silsesquioxane is chosen from polymethylsilsesquioxanes, which are silsesquioxanes such that R is a methyl group.

The polymethylsilsesquioxanes may comprise, for example, less than about 500 units T and preferably from about 50 to about 500 units T.

Not all polymethylsilsesquioxanes are film-forming. For example, the polymethylsilsesquioxanes such as Tospearl™ from Toshiba or KMP 590 from Shin-Etsu are highly insoluble in oils and, as a result, are inefficient film-forming agents. The molecular mass of these polymethylsilsesquioxanes is difficult to determine; they generally contain one thousand or more than one thousand units T.

An example of a polymethylsilsesquioxane that may be used according to the invention is Belsil PMS MK (also known as MK resin) available from Wacker Chemie. Polymethylsilsesquioxane is a polymer mainly consisting of CH₃SiO_{3/2} repeating units (units T) and also possibly containing up to about 1% (on a weight or molar basis) of (CH₃)₂SiO_{2/2} (units D).

The polymethylsilsesquioxanes that are suitable for use in the present invention comprise KR-220L, available from Shin-Etsu. The structure of KR-220L consists essentially of silicone units T (CH₃SiO_{3/2}) with Si—OH or silanol end units. There are no units D.

The polymethylsilsesquioxane KR-242A has a structure containing about 98% of methyl units T and about 2% of dimethyl units D, with Si—OH or silanol end units, and KR-251 has a structure containing about 88% of methyl units T and about 12% of dimethyl units D, with Si—OH or silanol end units; both are available from Shin-Etsu.

In one embodiment of the invention, the silicone resin is soluble or dispersible in silicone oils or volatile organic liquids. In one embodiment, the silicone resin is solid at 25° C.

In one embodiment, the silicone resin may have a molecular mass ranging from 1000 to 10 000 grams/mol. In one embodiment, the resin is present in the composition in an amount ranging from 0.5% to 20% by weight and preferably in an amount of 1% to 10% by weight relative to the total weight of the composition.

In one embodiment of the invention, the silicone resin is chosen from combinations of units M, D, T and Q, containing at least two units chosen from M, D, T and Q satisfying the relationship RₙSiO₍₄₋ₙ₎, in which n has a value ranging from 1.0 to 1.50. Certain resins of this type are described in U.S. Pat. No. 6,074,654.

In another embodiment, the film-forming silicone resin is a copolymer, in which at least one unit of the copolymer is chosen from the silicone units M, D, T and Q, and in which at least one additional unit of the copolymer is chosen from esters. The film-forming silicone resin may be chosen, for example, from diisostearoyltrimethylolpropane siloxysilicates, such as SF 1318 available from GE Silicones.

e) Silicone-based polyamide copolymers of the polyorganosiloxane type, such as those described in documents U.S. Pat. No. 5,874,069, U.S. Pat. No. 5,919,441, U.S. Pat. No. 6,051,216 and U.S. Pat. No. 5,981,680.

According to the invention, these silicone-based polymers may belong to the following two families:
1) polyorganosiloxanes comprising at least two groups capable of establishing hydrogen interactions, these two groups being located in the polymer chain; and/or
2) polyorganosiloxanes comprising at least two groups capable of establishing hydrogen interactions, these two groups being located on grafts or branches.

The polymers comprising two groups capable of establishing hydrogen interactions in the polymer chain may be polymers comprising at least one unit corresponding to formula (XXII): 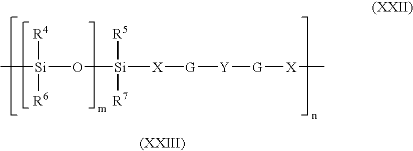
in which:

1) R⁴, R⁵, R⁶ and R⁷, which may be identical or different, represent a group chosen from:
linear, branched or cyclic, saturated or unsaturated, C₁ to C₄₀ hydrocarbon-based groups, possibly containing in their chain one or more oxygen, sulfur and/or nitrogen atoms, and possibly being partially or totally substituted with fluorine atoms,
C₆ to C₁₀ aryl groups, optionally substituted with one or more C₁ to C₄ alkyl groups,
polyorganosiloxane chains possibly containing one or more oxygen, sulfur and/or nitrogen atoms,

2) the groups X, which may be identical or different, represent a linear or branched C₁ to C₃₀ alkylenediyl group, possibly containing in its chain one or more oxygen and/or nitrogen atoms;

3) Y is a saturated or unsaturated, C₁ to C₅₀ linear or branched divalent alkylene, arylene, cycloalkylene, alkylarylene or arylalkylene group, possibly comprising one or more oxygen, sulfur and/or nitrogen atoms, and/or bearing as substituent one of the following atoms or groups of atoms: fluorine, hydroxyl, C₃ to C₈ cycloalkyl, C₁ to C₄₀ alkyl, C₅ to C₁₀ aryl, phenyl optionally substituted with 1 to 3 C₁ to C₃ alkyl groups, C₁ to C₃ hydroxyalkyl and C₁ to C₆ aminoalkyl; or

4) Y represents a group corresponding to formula (XXIII): 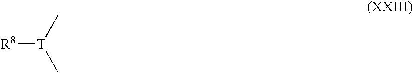
in which
T represents a linear or branched, saturated or unsaturated, C₃ to C₂₄ trivalent or tetravalent hydrocarbon-based group optionally substituted with a polyorganosiloxane chain, and possibly containing one or more atoms chosen from O, N and S, or T represents a trivalent atom chosen from N, P and Al, and
R⁸ represents a linear or branched C₁ to C₅₀ alkyl group or a polyorganosiloxane chain, possibly comprising one or more ester, amide, urethane, thiocarbamate, urea, thiourea and/or sulfonamide groups, which may possibly be linked to another chain of the polymer;

5) the groups G, which may be identical or different, represent divalent groups chosen from: 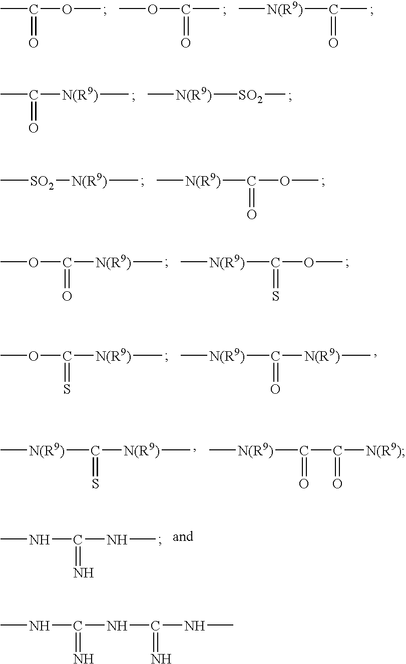
in which R⁹ represents a hydrogen atom or a linear or branched C₁ to C₂₀ alkyl group, on condition that at least 50% of the groups R⁹ of the polymer represent a hydrogen atom and that at least two of the groups G of the polymer are a group other than:

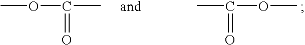

6) n is an integer ranging from 2 to 500 and preferably from 2 to 200, and m is an integer ranging from 1 to 1000, preferably from 1 to 700 and better still from 6 to 200.

According to the invention, 80% of the groups R⁴, R⁵, R⁶ and R⁷ of the polymer are preferably chosen from methyl, ethyl, phenyl and 3,3,3-trifluoropropyl groups.

According to the invention, Y can represent various divalent groups, furthermore optionally comprising one or two free valencies to establish bonds with other moieties of the polymer or copolymer. Preferably, Y represents a group chosen from:

a) linear C₁ to C₂₀ and preferably C₁ to C₁₀ alkylene groups,

b) C₃₀ to C₅₆ branched alkylene groups possibly comprising rings and unconjugated unsaturations,

c) C₅-C₆ cycloalkylene groups,

d) phenylene groups optionally substituted with one or more C₁ to C₄₀ alkyl groups,

e) C₁ to C₂₀ alkylene groups comprising from 1 to 5 amide groups,

f) C₁ to C₂₀ alkylene groups comprising one or more substituents chosen from hydroxyl, C₃ to C₈ cycloalkane, C₁ to C₃ hydroxyalkyl and C₁ to C₆ alkylamine groups,

g) polyorganosiloxane chains of formula (XXIV): 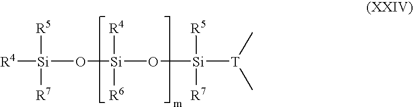
in which R⁴, R⁵, R⁶, R⁷, T and m are as defined above, and

h) polyorganosiloxane chains of formula (XXV):

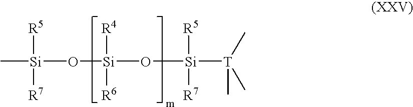

The polyorganosiloxanes of the second family may be polymers comprising at least one unit corresponding to formula (XXVI): 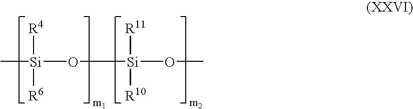
in which:
R⁴ and R⁶, which may be identical or different, are as defined above for formula (XXII),
R¹⁰ represents a group as defined above for R⁴ and R⁶, or represents a group of formula —X-G-R¹² in which X and G are as defined above for formula (XXII) and R¹² represents a hydrogen atom or a linear, branched or cyclic, saturated or unsaturated, C₁ to C₅₀ hydrocarbon-based group optionally comprising in its chain one or more atoms chosen from O, S and N, optionally substituted with one or more fluorine atoms and/or one or more hydroxyl groups, or a phenyl group optionally substituted with one or more C₁ to C₄ alkyl groups,
R¹¹ represents a group of formula —X-G-R⁹ in which X, G and R¹² are as defined above,
m₁ is an integer ranging from 1 to 998, and
m₂ is an integer ranging from 2 to 500.

According to the invention, the polymer used may be a homopolymer, that is to say a polymer comprising several identical units, in particular units of formula (XXII) or of formula (XXVI).

According to the invention, it is also possible to use a polymer consisting of a copolymer comprising several different units of formula (XXII), that is to say a polymer in which at least one of the groups R⁴, R⁵, R⁶, R⁷, X, G, Y, m and n is different in one of the units. The copolymer may also be formed from several units of formula (XXVI), in which at least one of the groups R⁴, R⁶, R¹⁰, R¹¹, m₁ and m₂ is different in at least one of the units.

It is also possible to use a copolymer comprising at least one unit of formula (XXII) and at least one unit of formula (XXVI), the units of formula (XXII) and the units of formula (XXVI) possibly being identical to or different from each other.

According to one variant, it is also possible to use a copolymer furthermore comprising at least one hydrocarbon-based unit comprising two groups capable of establishing hydrogen interactions, chosen from ester, amide, sulfonamide, carbamate, thiocarbamate, urea, urethane, thiourea, oxamido, guanidino and biguanidino groups, and combinations thereof.

These copolymers may be block copolymers or grafted copolymers.

f) Linear block ethylenic polymers

The composition according to the invention may contain, as film-forming agent, a linear block ethylenic polymer, referred to hereinbelow as a “block polymer”, the particular structure of which being as described below.

The term “block” polymer means a polymer comprising at least two different blocks and preferably at least three different blocks.

The polymer is a polymer of linear structure. In contrast, a polymer of non-linear structure is, for example, a polymer of branched, star or grafted structure, or the like.

Advantageously, the block polymer is free of styrene. The term “polymer free of styrene” means a polymer containing less than 10% by weight, preferably less than 5% by weight, better still less than 2% by weight and better still less than 1% by weight of styrene monomer, for instance styrene, styrene derivatives such as methylstyrene, chlorostyrene or chloromethylstyrene, or even containing no styrene monomer, relative to the total weight of the polymer.

In particular, the block polymer comprises at least one first block and at least one second block that have different glass transition temperatures (Tg), the said first and second blocks being linked together via an intermediate block comprising at least one constituent monomer of the first block and at least one constituent monomer of the second block.

The term “at least one block” means one or more blocks.

The intermediate block is a block comprising at least one constituent monomer of the first block and at least one constituent monomer of the second block of the polymer allowing these blocks to be “compatibilized”.

It is pointed out that, in the text hereinabove and hereinbelow, the terms “first” and “second” blocks do not in any way condition the order of the said blocks in the structure of the block polymer.

Advantageously, the first and second blocks of the block polymer are mutually incompatible.

The term “mutually incompatible blocks” means that the mixture formed from the polymer corresponding to the first block and of the polymer corresponding to the second block is not miscible in the organic liquid that is in major amount by weight contained in the liquid fatty phase, at room temperature (25° C.) and atmospheric pressure (10⁵ Pa), for a content of the polymer mixture of greater than or equal to 5% by weight, relative to the total weight of the mixture (polymers and solvent), it being understood that:

i) the said polymers are present in the mixture in a content such that the respective weight ratio ranges from 10/90 to 90/10, and that

ii) each of the polymers corresponding to the first and second blocks has an average (weight-average or number-average) molar mass equal to that of the block polymer ±15%.

When the composition comprises a liquid fatty phase comprising a mixture of organic liquids, and in the event that two or more organic liquids are present in identical mass proportions, the said polymer mixture is immiscible in at least one of them.

When the liquid fatty phase comprises only one organic liquid, this liquid is the predominant organic liquid.

In particular, the block polymer comprises no silicon atoms in its backbone. The term “backbone” means the main chain of the polymer, as opposed to the pendent side chains.

In particular, the block polymer is not soluble in water or in a mixture of water and linear or branched lower monoalcohols containing from 2 to 5 carbon atoms, for instance ethanol, isopropanol or n-propanol, without modifying the pH, at an active material content of at least 1% by weight, at room temperature (25° C.).

In particular, the block polymer is not an elastomer.

The term “non-elastomeric polymer” means a polymer which, when it is subjected to a constraint intended to stretch it (for example by 30% relative to its initial length), does not return to a length substantially identical to its initial length when the constraint ceases.

More specifically, the term “non-elastomeric polymer” denotes a polymer with an instantaneous recovery Rᵢ<50% and a delayed recovery R₂ₕ<70% after having been subjected to a 30% elongation. Preferably, Rᵢ is <30% and R₂ₕ<50%.

i) Recovery Test

More specifically, the non-elastomeric nature of the polymer is determined according to the following protocol:

A polymer film is prepared by pouring a solution of the polymer in a Teflon-coated mould, followed by drying for 7 days in an environment conditioned at 23±5° C. and 50±10% relative humidity.

A film about 100 μm thick is thus obtained, from which are cut rectangular specimens (for example using a punch) 15 mm wide and 80 mm long.

This sample is subjected to a tensile stress using a machine sold under the reference Zwick, under the same temperature and humidity conditions as for the drying.

The specimens are pulled at a speed of 50 mm/min and the distance between the jaws is 50 mm, which corresponds to the initial length (I₀) of the specimen.

The instantaneous recovery Rᵢ is determined in the following manner:
the specimen is pulled by 30% (εₘₐₓ), i.e. about 0.3 times its initial length (I₀)
the constraint is released by applying a return speed equal to the tensile speed, i.e. 50 mm/min, and the residual elongation of the specimen is measured as a percentage, after returning to zero constraint (εᵢ).

The percentage instantaneous recovery (Rᵢ) is given by the following formula:
Rᵢ=(εₘₐₓ−εᵢ)/εₘₐₓ)×100

To determine the delayed recovery, the percentage residual elongation of the specimen (ε₂ₕ) is measured 2 hours after returning to zero constraint.

The percentage delayed recovery (R₂ₕ) is given by the following formula:
R₂ₕ=(εₘₐₓ−ε₂ₕ)/εₘₐₓ)×100

Purely as a guide, a polymer according to one embodiment of the invention has an instantaneous recovery Rᵢ of 10% and a delayed recovery R₂ₕ of 30%.

Advantageously, the block polymer has a polydispersity index I of greater than 2, for example ranging from 2 to 9, preferably greater than or equal to 2.5, for example ranging from 2.5 to 8 and better still greater than or equal to 2.8, and especially ranging from 2.8 to 6.

The polydispersity index I of the block polymer is equal to the ratio of the weight-average mass Mw to the number-average mass Mn.

The weight-average molar mass (Mw) and number-average molar mass (Mn) are determined by gel permeation liquid chromatography (THF solvent, calibration curve established with linear polystyrene standards, refractometric detector).

The weight-average mass (Mw) of the block polymer is preferably less than or equal to 300 000; it ranges, for example, from 35 000 to 200 000 and better still from 45 000 to 150 000.

The number-average mass (Mn) of the block polymer is preferably less than or equal to 70 000; it ranges, for example, from 10 000 to 60 000 and better still from 12 000 to 50 000.

Each block of the block polymer is derived from one type of monomer or from several different types of monomer.

This means that each block may consist of a homopolymer or a copolymer; this copolymer constituting the block may in turn be random or alternating.

Advantageously, the intermediate block comprising at least one constituent monomer of the first block and at least one constituent monomer of the second block of the block polymer is a random polymer.

Preferably, the intermediate block is derived essentially from constituent monomers of the first block and of the second block.

The term “essentially” means at least 85%, preferably at least 90%, better still 95% and even better still 100%.

Advantageously, the intermediate block has a glass transition temperature Tg that is between the glass transition temperatures of the first and second blocks.

The glass transition temperatures indicated for the first and second blocks may be theoretical Tg values determined from the theoretical Tg values of the constituent monomers of each of the blocks, which may be found in a reference manual such as the Polymer Handbook, 3rd Edition, 1989, John Wiley, according to the following relationship, known as Fox's law:
1/Tg=Σᵢ( [symbol] ᵢ/Tgᵢ), [symbol]
ᵢ being the mass fraction of the monomer i in the block under consideration and Tgᵢ being the glass transition temperature of the homopolymer of the monomer i.

Unless otherwise indicated, the Tg values indicated for the first and second blocks in the present patent application are theoretical Tg values.

The difference between the glass transition temperatures of the first and second blocks is generally greater than 10° C., preferably greater than 20° C. and better still greater than 30° C.

ii) Polymer Blocks

In particular, the first block of the block polymer may be chosen from:
a) a block with a Tg of greater than or equal to 40° C.,
b) a block with a Tg of less than or equal to 20° C.,
c) a block with a Tg of between 20 and 40° C., and the second block can be chosen from a category a), b) or c) different from the first block.

In the present invention, the expression:

“between . . . and . . . ” is intended to denote a range of values for which the limits mentioned are excluded, and

“from . . . to . . . ” and “ranging from . . . to . . . ” are intended to denote a range of values for which the limits are included.

a) Block with a Tg of Greater than or Equal to 40° C.

The block with a Tg of greater than or equal to 40° C. has, for example, a Tg ranging from 40 to 150° C., preferably greater than or equal to 50° C., for example ranging from 50° C. to 120° C. and better still greater than or equal to 60° C., for example ranging from 60° C. to 120° C.

The block with a Tg of greater than or equal to 40° C. may be a homopolymer or a copolymer.

In the case where this block is a homopolymer, it is derived from monomers which are such that the homopolymers prepared from these monomers have glass transition temperatures of greater than or equal to 40° C. This first block may be a homopolymer consisting of only one type of monomer (for which the Tg of the corresponding homopolymer is greater than or equal to 40° C.).

In the case where the first block is a copolymer, it may be totally or partially derived from one or more monomers, the nature and concentration of which are chosen such that the Tg of the resulting copolymer is greater than or equal to 40° C. The copolymer may comprise, for example:
monomers which are such that the homopolymers prepared from these monomers have Tg values of greater than or equal to 40° C., for example a Tg ranging from 40 to 150° C., preferably greater than or equal to 50° C., for example ranging from 50° C. to 120° C. and better still greater than or equal to 60° C., for example ranging from 60° C. to 120° C., and
monomers which are such that the homopolymers prepared from these monomers have Tg values of less than 40° C., chosen from monomers with a Tg of between 20 and 40° C. and/or monomers with a Tg of less than or equal to 20° C., for example a Tg ranging from −100 to 20° C., preferably less than 15° C., especially ranging from −80° C. to 15° C. and better still less than 10° C., for example ranging from −50° C. to 0° C., as described later.

The monomers whose homopolymers have a glass transition temperature of greater than or equal to 40° C. are chosen, preferably, from the following monomers, also known as the main monomers:
methacrylates of formula (XII):
   CH₂═C(CH₃)—COOR₁ (XII)
   in which R₁ represents a linear or branched unsubstituted alkyl group containing from 1 to 4 carbon atoms, such as a methyl, ethyl, propyl or isobutyl group or R₁ represents a C₄ to C₁₂ cycloalkyl group,
acrylates of formula (XIII):
   CH₂═CH—COOR₂ (XIII)
   in which R₂ represents a C₄ to C₁₂ cycloalkyl group such as isobornyl acrylate or a tert-butyl group,
(meth)acrylamides of formula (XIV): 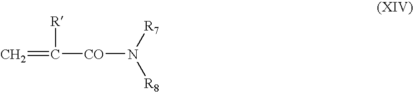
   in which:
R₇ and R₈, which may be identical or different, each represent a hydrogen atom or a linear or branched C₁ to C₁₂ alkyl group such as an n-butyl, t-butyl, isopropyl, isohexyl, isooctyl or isononyl group; or R₇ represents H and R₈ represents a 1,1-dimethyl-3-oxobutyl group, and
R′ denotes H or methyl,
and mixtures thereof.

Examples of monomers that may be mentioned include N-butylacrylamide, N-t-butylacrylamide, N-isopropylacrylamide, N,N-dimethylacrylamide and N,N-dibutylacrylamide,

Main monomers that are particularly advantageous are methyl methacrylate, isobutyl (meth)acrylate and isobornyl (meth)acrylate, and mixtures thereof.

b) Block with a Tg of Less Than or Equal to 20° C.

The block with a Tg of less than or equal to 20° C. has, for example, a Tg ranging from −100 to 20° C., preferably less than or equal to 15° C., especially ranging from −80° C. to 15° C. and better still less than or equal to 10° C., for example ranging from −50° C. to 0° C.

The block with a Tg of less than or equal to 20° C. may be a homopolymer or a copolymer.

In the case where this block is a homopolymer, it is derived from monomers which are such that the homopolymers prepared from these monomers have glass transition temperatures of less than or equal to 20° C. This second block may be a homopolymer consisting of only one type of monomer (for which the Tg of the corresponding homopolymer is less than or equal to 20° C.).

In the case where the block with a Tg of less than or equal to 20° C. is a copolymer, it may be totally or partially derived from one or more monomers, the nature and concentration of which are chosen such that the Tg of the resulting copolymer is less than or equal to 20° C.

It may comprise, for example
one or more monomers whose corresponding homopolymer has a Tg of less than or equal to 20° C., for example a Tg ranging from −100° C. to 20° C., preferably less than 15° C., especially ranging from −80° C. to 15° C. and better still less than 10° C., for example ranging from −50° C. to 0° C., and
one or more monomers whose corresponding homopolymer has a Tg of greater than 20° C., such as monomers with a Tg of greater than or equal to 40° C., for example a Tg ranging from 40 to 150° C., preferably greater than or equal to 50° C., for example ranging from 50° C. to 120° C. and better still greater than or equal to 60° C., for example ranging from 60° C. to 120° C. and/or monomers with a Tg of between 20 and 40° C., as described above.

In particular, the block with a Tg of less than or equal to 20° C. is a homopolymer.

The monomers whose homopolymer has a Tg of less than or equal to 20° C. are preferably chosen from the following monomers, or main monomers:
acrylates of formula (XV):
   CH₂═CHCOOR₃ (XV)
   R₃ representing a linear or branched C₁ to C₁₂ unsubstituted alkyl group, with the exception of the tert-butyl group, in which one or more hetero atoms chosen from O, N and S is (are) optionally intercalated,
methacrylates of formula (XVI):
   CH₂═C(CH₃)—COOR₄ (XVI)
   R₄ representing a linear or branched C₆ to C₁₂ unsubstituted alkyl group, in which one or more hetero atoms chosen from O, N and S is (are) optionally intercalated;
vinyl esters of formula (XVII):
   R₅—CO—O—CH═CH₂ (XVII)
   in which R₅ represents a linear or branched C₄ to C₁₂ alkyl group;
C₄ to C₁₂ alkyl vinyl ethers and alkyl ethers,
N—(C₄ to C₁₂)alkyl acrylamides, such as N-octylacrylamide,
and mixtures thereof.

The main monomers that are particularly preferred for the block with a Tg of less than or equal to 20° C. are alkyl acrylates whose alkyl chain contains from 1 to 10 carbon atoms, with the exception of the tert-butyl group, such as methyl acrylate, isobutyl acrylate and 2-ethylhexyl acrylate, and mixtures thereof.

c) Block with a Tg of Between 20 and 40° C.

The block with a Tg of between 20 and 40° C. may be a homopolymer or a copolymer.

In the case where this block is a homopolymer, it is derived from monomers (or main monomer) which are such that the homopolymers prepared from these monomers have glass transition temperatures of between 20 and 40° C. This first block may be a homopolymer, consisting of only one type of monomer (for which the Tg of the corresponding homopolymer ranges from 20° C. to 40° C.).

The monomers whose homopolymer has a glass transition temperature of between 20 and 40° C. are preferably chosen from n-butyl methacrylate, cyclodecyl acrylate, neopentyl acrylate and isodecylacrylamide, and mixtures thereof.

In the case where the block with a Tg of between 20 and 40° C. is a copolymer, it is totally or partially derived from one or more monomers (or main monomer) whose nature and concentration are chosen such that the Tg of the resulting copolymer is between 20 and 40° C.

Advantageously, the block with a Tg of between 20 and 40° C. is a copolymer totally or partially derived from:
main monomers whose corresponding homopolymer has a Tg of greater than or equal to 40° C., for example a Tg ranging from 40° C. to 150° C., in particular greater than or equal to 50° C., for example ranging from 50 to 120° C. and better still greater than or equal to 60° C., for example ranging from 60° C. to 120° C., as described above, and/or
main monomers whose corresponding homopolymer has a Tg of less than or equal to 20° C., for example a Tg ranging from −100 to 20° C., in particular less than or equal to 15° C., especially ranging from −80° C. to 15° C. and in particular less than or equal to 10° C., for example ranging from −50° C. to 0° C., as described above,
   the said monomers being chosen such that the Tg of the copolymer forming the first block is between 20 and 40° C.

Such main monomers are chosen, for example, from methyl methacrylate, isobornyl acrylate and methacrylate, butyl acrylate and 2-ethylhexyl acrylate, and mixtures thereof.

More particularly, the proportion of the second block with a Tg of less than or equal to 20° C. ranges from 10% to 85% by weight, better still from 20% to 70% and even better still from 20% to 50% by weight of the polymer.

However, each of the blocks may contain in small proportion at least one constituent monomer of the other block.

Thus, the first block may contain at least one constituent monomer of the second block, and vice versa.

Each of the first and/or second blocks of the block polymer may comprise, in addition to the monomers indicated above, one or more other monomers known as additional monomers, which are different from the main monomers mentioned above.

The nature and amount of this or these additional monomer(s) are chosen such that the block in which they are present has the desired glass transition temperature.

iii) Additional Monomer

This additional monomer is chosen, for example, from:
hydrophilic monomers such as:
   ethylenically unsaturated monomers comprising at least one carboxylic or sulfonic acid function, for instance:
      acrylic acid, methacrylic acid, crotonic acid, maleic anhydride, itaconic acid, fumaric acid, maleic acid, acrylamidopropanesulfonic acid, vinylbenzoic acid, vinylphosphoric acid, and salts thereof,
   ethylenically unsaturated monomers comprising at least one tertiary amine function, for instance
      2-vinylpyridine, 4-vinylpyridine, dimethylaminoethyl methacrylate, diethylaminoethyl methacrylate and dimethylaminopropylmethacrylamide, and salts thereof,
      methacrylates of formula (XVIII):
         CH₂═C(CH₃)—COOR₆ (XVIII)
         in which R₆ represents a linear or branched alkyl group containing from 1 to 4 carbon atoms, such as a methyl, ethyl, propyl or isobutyl group, the said alkyl group being substituted with one or more substituents chosen from hydroxyl groups (for instance 2-hydroxypropyl methacrylate and 2-hydroxyethyl methacrylate) and halogen atoms (Cl, Br, I or F), such as trifluoroethyl methacrylate,
methacrylates of formula (XIX):
   CH₂═C(CH₃)—COOR₉ (XIX)
   in which R₉ represents a linear or branched C₆ to C₁₂ alkyl group in which one or more hetero atoms chosen from O, N and S is (are) optionally intercalated, the said alkyl group being substituted with one or more substituents chosen from hydroxyl groups and halogen atoms (Cl, Br, I or F);
acrylates of formula (XX):
   CH₂═CHCOOR₁₀ (XX)
   in which R₁₀ represents a linear or branched C₁ to C₁₂ alkyl group substituted with one or more substituents chosen from hydroxyl groups and halogen atoms (Cl, Br, I and F), such as 2-hydroxypropyl acrylate and 2-hydroxyethyl acrylate, or R₁₀ represents a C₁ to C₁₂ alkyl-O—POE (polyoxyethylene) with repetition of the oxyethylene unit 5 to 30 times, for example methoxy-POE, or R₈ represents a polyoxyethylenated group comprising from 5 to 30 ethylene oxide units
ethylenically unsaturated monomers comprising one or more silicon atoms, such as methacryloxypropyltrimethoxysilane and methacryloxypropyltris(trimethylsiloxy)silane,
and mixtures thereof.

Additional monomers that are particularly preferred are acrylic acid, methacrylic acid and trifluoroethyl methacrylate, and mixtures thereof.

According to one preferred embodiment, the block polymer is a non-silicone polymer, i.e. a polymer free of silicon atoms.

This or these additional monomer(s) generally represent(s) an amount of less than or equal to 30% by weight, for example from 1% to 30% by weight, preferably from 5% to 20% by weight and more preferably from 7% to 15% by weight, relative to the total weight of the first and/or second blocks.

In particular, each of the first and second blocks comprises at least one monomer chosen from (meth)acrylic acid esters, and optionally at least one monomer chosen from (meth)acrylic acid, and mixtures thereof.

Advantageously, each of the first and second blocks of the block polymer is totally derived from at least one monomer chosen from acrylic acid and (meth)acrylic acid esters, and optionally at least one monomer chosen from (meth)acrylic acid, and mixtures thereof.

iv) Preparation Process

The block polymer may be obtained by free-radical solution polymerization according to the following preparation process:
a portion of the polymerization solvent is introduced into a suitable reactor and heated until the adequate temperature for the polymerization is reached (typically between 60 and 120° C.),
once this temperature is reached, the constituent monomers of the first block are introduced in the presence of part of the polymerization initiator,
after a time T corresponding to a maximum degree of conversion of 90%, the constituent monomers of the second block and the rest of the initiator are introduced,
the mixture is left to react for a time T′ (ranging from 3 to 6 hours), after which the mixture is cooled to room temperature,
the polymer dissolved in the polymerization solvent is obtained.

The term “polymerization solvent” means a solvent or a mixture of solvents. The polymerization solvent may be chosen especially from ethyl acetate, butyl acetate, alcohols such as isopropanol or ethanol, and aliphatic alkanes such as isododecane, and mixtures thereof. Preferably, the polymerization solvent is a mixture of butyl acetate and isopropanol or isododecane.

According to a first embodiment, the block polymer comprises a first block with a Tg of greater than or equal to 40° C., as described above in a) and a second block with a Tg of less than or equal to 20° C., as described above in b).

In particular, the first block with a Tg of greater than or equal to 40° C. is a copolymer derived from monomers which are such that the homopolymer prepared from these monomers has a glass transition temperature of greater than or equal to 40° C., such as the monomers described above.

Advantageously, the second block with a Tg of less than or equal to 20° C. is a homopolymer derived from monomers which are such that the homopolymer prepared from these monomers has a glass transition temperature of less than or equal to 20° C., such as the monomers described above.

In particular, the proportion of the block with a Tg of greater than or equal to 40° C. ranges from 20% to 90%, better still from 30% to 80% and even better still from 50% to 70% by weight of the polymer.

In particular, the proportion of the block with a Tg of less than or equal to 20° C. ranges from 5% to 75%, preferably from 15% to 50% and better still from 25% to 45% by weight of the polymer.

Advantageously, the block polymer may comprise:
a first block with a Tg of greater than or equal to 40° C., for example ranging from 85 to 115° C., which is an isobornyl acrylate/isobutyl methacrylate copolymer,
a second block with a Tg of less than or equal to 20° C., for example ranging from −85 to −55° C., which is a 2-ethylhexyl acrylate homopolymer, and
an intermediate block, which is an isobornyl acrylate/isobutyl methacrylate/2-ethylhexyl acrylate random copolymer.

According to another embodiment, the block polymer comprises a first block having a glass transition temperature (Tg) of between 20 and 40° C., in accordance with the blocks described in c) and a second block having a glass transition temperature of less than or equal to 20° C., as described above in b) or a glass transition temperature of greater than or equal to 40° C., as described in a) above.

In particular, the proportion of the first block with a Tg of between 20 and 40° C. ranges from 10% to 85%, better still from 30% to 80% and even better still from 50% to 70% by weight of the polymer.

When the second block is a block with a Tg of greater than or equal to 40° C., it is preferably present in a proportion ranging from 10% to 85% by weight, better still from 20% to 70% and even better still from 30% to 70% by weight of the polymer.

When the second block is a block with a Tg of less than or equal to 20° C., it is preferably present in a proportion ranging from 10% to 85% by weight, better still from 20% to 70% and even better still from 20% to 50% by weight of the polymer.

In particular, the first block with a Tg of between 20 and 40° C. is a copolymer derived from monomers which are such that the corresponding homopolymer has a Tg of greater than or equal to 40° C., and from monomers which are such that the corresponding homopolymer has a Tg of less than or equal to 20° C.

The second block with a Tg of less than or equal to 20° C. or with a Tg of greater than or equal to 40° C. is advantageously a homopolymer.

According to a first variant, the block polymer comprises:
a first block with a Tg of between 20 and 40° C., for example with a Tg of 21 to 39° C., which is a copolymer comprising isobornyl acrylate/isobutyl methacrylate/2-ethylhexyl acrylate,
a second block with a Tg of less than or equal to 20° C., for example ranging from −65 to −35° C., which is a homopolymer of methyl methacrylate, and
an intermediate block which is an isobornyl acrylate/isobutyl methacrylate/2-ethylhexyl acrylate random copolymer.

According to another variant, the block polymer may comprise:
a first block with a Tg of greater than or equal to 40° C., for example ranging from 85 to 115° C., which is an isobornyl methacrylate/isobutyl methacrylate copolymer,
a second block with a Tg of less than or equal to 20° C., for example ranging from −35 to −5° C., which is an isobutyl acrylate homopolymer, and
an intermediate block, which is an isobornyl methacrylate/isobutyl methacrylate/isobutyl acrylate random copolymer.

According to yet another variant, the block polymer may comprise:
a first block with a Tg of greater than or equal to 40° C., for example ranging from 60 to 90° C., which is an isobornyl acrylate/isobutyl methacrylate copolymer,
a second block with a Tg of less than or equal to 20° C., for example ranging from −35 to −5° C., which is an isobutyl acrylate homopolymer, and
an intermediate block, which is an isobornyl acrylate/isobutyl methacrylate/isobutyl acrylate random copolymer.

g) the products of the reaction between a silica derivative and a polydiorganosiloxane bearing silanol end groups, as described in U.S. Pat. No. 5,162,410, U.S. Pat. No. 330,747 and U.S. Pat. No. 5,451,610, the content of which is incorporated into the present patent application by reference. Such products are especially those sold under the reference Bio-PSA by Dow Corning, for example the product of this range referenced 7-4405.

According to the invention, the film-forming polymer may be a solid that is insoluble in the fatty phase of the composition at room temperature, for example at approximately 25° C. The polymer is also insoluble in the fatty phase at its softening point, unlike a wax, even of polymeric origin, which is soluble in the liquid organic phase (or fatty phase) at its melting point. In this sense, the polymer is not a wax.

1) Polymers

The composition according to the invention may comprise at least one stable dispersion of essentially spherical polymer particles of one or more polymers, in a physiologically acceptable fatty phase.

These dispersions may especially be in the form of polymer nanoparticles in stable dispersion in the said liquid organic phase. The nanoparticles preferably have a mean size of between 5 and 800 nm and better still between 50 and 500 nm. However, it is possible to obtain polymer particles ranging up to 1 μm in size.

In particular, the polymer particles in dispersion are insoluble in water-soluble alcohols, for instance ethanol.

The polymers in dispersion that may be used in the composition of the invention preferably have a molecular weight of about from 2000 to 10 000 000 g/mol and a Tg of from −100° C. to 300° C., better still from −50° C. to 100° C. and preferably from −10° C. to 50° C.

It is possible to use film-forming polymers preferably having a low Tg, of less than or equal to skin temperature and especially less than or equal to 40° C.

Among the film-forming polymers that may be mentioned are acrylic or vinyl free-radical homopolymers or copolymers, preferably with a Tg of less than or equal to 40° C. and especially ranging from −10° C. to 30° C., used alone or as a mixture.

The term “free-radical polymer” means a polymer obtained by polymerization of unsaturated and especially ethylenic monomers, each monomer being capable of homopolymerizing (unlike polycondensates). The free-radical polymers may especially be vinyl polymers or copolymers, especially acrylic polymers.

The acrylic polymers may result from the polymerization of ethylenically unsaturated monomers containing at least one acid group and/or esters of these acid monomers and/or amides of these acids.

Monomers bearing an acid group that may be used include α,β-ethylenic unsaturated carboxylic acids such as acrylic acid, methacrylic acid, crotonic acid, maleic acid or itaconic acid. (Meth)acrylic acid and crotonic acid are preferably used, and more preferably (meth)acrylic acid.

The acid monomer esters are advantageously chosen from (meth)acrylic acid esters (also known as (meth)acrylates), for instance alkyl (meth)acrylates, in particular of a C₁-C₂₀ and preferably C₁-C₈ alkyl, aryl (meth)acrylates, in particular of a C₆-C₁₀ aryl, and hydroxyalkyl (meth)acrylates, in particular of a C₂-C₆ hydroxyalkyl. Alkyl (meth)acrylates that may be mentioned include methyl, ethyl, butyl, isobutyl, 2-ethylhexyl and lauryl (meth)acrylate. Hydroxyalkyl (meth)acrylates that may be mentioned include hydroxyethyl (meth)acrylate and 2-hydroxypropyl (meth)acrylate. Aryl (meth)acrylates that may be mentioned include benzyl or phenyl acrylate.

The (meth)acrylic acid esters that are particularly preferred are the alkyl (meth)acrylates.

Free-radical polymers that are preferably used include copolymers of (meth)acrylic acid and of alkyl (meth)acrylate, especially of a C₁-C₄ alkyl. Methyl acrylates optionally copolymerized with acrylic acid may more preferentially be used.

Amides of the acid monomers that may be mentioned include (meth)acrylamides, especially N-alkyl(meth)acrylamides, in particular of a C₂-C₁₂ alkyl, such as N-ethylacrylamide, N-t-butylacrylamide and N-octylacrylamide; N-di(C₁-C₄)alkyl(meth)acrylamides.

The acrylic polymers may also result from the polymerization of ethylenically unsaturated monomers containing at least one amine group, in free form or in partially or totally neutralized form, or alternatively in partially or totally quaternized form. Such monomers may be, for example, dimethylaminoethyl (meth)acrylate, dimethylaminoethylmethacrylamide, vinylamine, vinylpyridine or diallyldimethylammonium chloride.

The vinyl polymers may also result from the homopolymerization or copolymerization of at least one monomer chosen from vinyl esters and styrene monomers. In particular, these monomers may be polymerized with acid monomers and/or esters thereof and/or amides thereof, such as those mentioned previously. Examples of vinyl esters that may be mentioned include vinyl acetate, vinyl propionate, vinyl neodecanoate, vinyl pivalate, vinyl benzoate and vinyl t-butylbenzoate. Styrene monomers that may be mentioned include styrene and α-methylstyrene.

The list of monomers given is not limiting, and it is possible to use any monomer known to those skilled in the art included in the categories of acrylic and vinyl monomers (including monomers modified with a silicone chain).

As other vinyl monomers that may be used, mention may also be made of:
N-vinylpyrrolidone, N-vinylcaprolactam, vinyl-N—(C₁-C₆) alkylpyrroles, vinyloxazoles, vinylthiazoles, vinyl-pyrimidines and vinylimidazoles,
olefins such as ethylene, propylene, butylene, iso-prene or butadiene.

The vinyl polymer may be crosslinked with one or more difunctional monomers especially comprising at least two ethylenic unsaturations, such as ethylene glycol dimethacrylate or diallyl phthalate.

In a non-limiting manner, the polymers in dispersion of the invention may be chosen from the following polymers or copolymers: polyurethanes, polyurethane-acrylics, polyureas, polyurea-polyurethanes, polyester-polyurethanes, polyether-polyurethanes, polyesters, polyesteramides, alkyds; acrylic and/or vinyl polymers or copolymers; acrylic-silicone copolymers; polyacrylamides; silicone polymers, for instance silicone polyurethanes or silicone acrylics, and fluoro polymers, and mixtures thereof.

The polymer(s) in dispersion in the fatty phase may represent from 5% to 40% of the weight of solids in the composition.

2) Stabilizer

According to one embodiment, the polymer particles in dispersion are surface-stabilized with a stabilizer that is solid at room temperature. In this case, the amount of solids in the dispersion represents the total amount of polymer+stabilizer, given that the amount of polymer cannot be less than 5%.

The polymer particles are in particular surface-stabilized by means of a stabilizer that may be a block polymer, a grafted polymer and/or a random polymer, alone or as a mixture. The stabilization may take place by any known means, and in particular by direct addition of the stabilizing polymer during the polymerization.

The stabilizer may also be present in the mixture before polymerization of the polymer. However, it is also possible to add it continuously, especially when the monomers are also added continuously.

2-30% by weight and preferably 5-20% by weight of stabilizer may be used relative to the initial monomer mixture.

When a grafted polymer and/or a block polymer is used as stabilizer, the synthesis solvent is chosen such that at least some of the grafts or blocks of the said polymer-stabilizer are soluble in the said solvent, the rest of the grafts or blocks being insoluble therein. The polymer-stabilizer used during the polymerization should be soluble, or dispersible, in the synthesis solvent. Furthermore, a stabilizer whose insoluble blocks or grafts have a certain affinity for the polymer formed during the polymerization is preferably chosen.

Among the grafted polymers that may be mentioned are silicone polymers grafted with a hydrocarbon-based chain; hydrocarbon-based polymers grafted with a silicone chain.

Thus, grafted-block or block copolymers comprising at least one block of polyorganosiloxane type and at least one block of a free-radical polymer, for instance grafted copolymers of acrylic/silicone type, may thus be used, which may be used especially when the non-aqueous medium contains silicone.

It is also possible to use grafted-block or block copolymers comprising at least one block of polyorganosiloxane type and at least one block of a polyether. The polyorganopolysiloxane block may especially be a polydimethylsiloxane or a poly(C₂-C₁₈)alkylmethylsiloxane; the polyether block may be a poly(C₂-C₁₈)alkylene, in particular polyoxyethylene and/or polyoxypropylene. In particular, dimethicone copolyols or (C₂-C₁₈)alkyldimethicone copolyols such as those sold under the name “Dow Corning 3225C” by the company Dow Corning, and lauryl methicones such as those sold under the name “Dow Corning Q2-5200” by the company Dow Corning, may be used.

Grafted-block or block copolymers that may also be mentioned include those comprising at least one block resulting from the polymerization of at least one ethylenic monomer containing one or more optionally conjugated ethylenic bonds, for instance ethylene or dienes such as butadiene and isoprene, and of at least one block of a vinyl polymer and better still a styrene polymer. When the ethylenic monomer comprises several optionally conjugated ethylenic bonds, the residual ethylenic unsaturations after the polymerization are generally hydrogenated. Thus, in a known manner, the polymerization of isoprene leads, after hydrogenation, to the formation of an ethylene-propylene block, and the polymerization of butadiene leads, after hydrogenation, to the formation of an ethylene-butylene block. Among these polymers that may be mentioned are block copolymers, especially of “diblock” or “triblock” type such as polystyrene/polyisoprene (SI), polystyrene/polybutadiene (SB) such as those sold under the name “Luvitol HSB” by BASF, of the type such as polystyrene/copoly(ethylene-propylene) (SEP) such as those sold under the name “Kraton” by Shell Chemical Co. or of the type such as polystyrene/copoly-(ethylene-butylene) (SEB). Kraton G1650 (SEBS), Kraton G1651 (SEBS), Kraton G1652 (SEBS), Kraton G1657X (SEBS), Kraton G1701X (SEP), Kraton G1702X (SEP), Kraton G1726X (SEB), Kraton D-1101 (SBS), Kraton D-1102 (SBS) and Kraton D-1107 (SIS) may be used in particular. The polymers are generally known as hydrogenated or non-hydrogenated diene copolymers.

Gelled Permethyl 99A-750, 99A-753-59 and 99A-753-58 (mixture of triblock and of star polymer), Versagel 5960 from Penreco (triblock+star polymer); OS129880, OS129881 and OS84383 from Lubrizol (styrene/methacrylate copolymer) may also be used.

As grafted-block or block copolymers comprising at least one block resulting from the polymerization of at least one ethylenic monomer containing one or more ethylenic bonds and of at least one block of an acrylic polymer, mention may be made of poly(methyl methacrylate)/polyisobutylene diblock or triblock copolymers or grafted copolymers containing a poly(methyl methacrylate) backbone and polyisobutylene grafts.

As grafted-block or block copolymers comprising at least one block resulting from the polymerization of at least one ethylenic monomer containing one or more ethylenic bonds and of at least one block of a polyether such as a C₂-C₁₈ polyalkylene (especially polyethylene and/or polyoxypropylene), mention may be made of polyoxyethylene/polybutadiene or polyoxyethylene/polyisobutylene diblock or triblock copolymers.

When a random polymer is used as stabilizer, it is chosen such that it has a sufficient amount of groups making it soluble in the intended synthesis solvent.

Copolymers based on alkyl acrylates or methacrylates derived from C₁-C₄ alcohols and on alkyl acrylates or methacrylates derived from C₈-C₃₀ alcohols may thus be used. Mention may be made in particular of stearyl methacrylate/methyl methacrylate copolymer.

When the synthesis solvent of the polymer is apolar, it is preferable to choose as stabilizer a polymer that provides the fullest possible coverage of the particles, several polymer-stabilizer chains then being absorbed onto a particle of polymer obtained by polymerization.

In this case, it is preferred to use as stabilizer either a grafted polymer or a block polymer, so as to have better interfacial activity. Specifically, blocks or grafts that are insoluble in the synthesis solvent provide bulkier coverage at the surface of the particles.

When the synthesis solvent comprises at least one silicone oil, the stabilizer is preferably chosen from the group consisting of grafted-block or block copolymers comprising at least one block of polyorganosiloxane type and at least one block of a free-radical polymer or of a polyether or of a polyester, for instance polyoxypropylene and/or oxyethylene blocks.

When the synthesis solvent does not comprise any silicone oil, the stabilizer is preferably chosen from the group consisting of:

a) grafted-block or block copolymers comprising at least one block of polyorganosiloxane type and at least one block of a free-radical polymer or of a polyether or a polyester,

b) copolymers of alkyl acrylates or methacrylates derived from C₁-C₄ alcohols and of alkyl acrylates or methacrylates derived from C₈-C₃₀ alcohols,

c) grafted-block or block copolymers comprising at least one block resulting from the polymerization of at least one ethylenic monomer containing conjugated ethylenic bonds,

and at least one block of a vinyl or acrylic polymer or of a polyether or of a polyester, or mixtures thereof.

Diblock polymers are preferably used as stabilizer.

A film-forming polymer that is liposoluble or in dispersion in a fatty phase may also be used in an amount ranging from 0.01% to 20% (as active material), for instance from 1% to 10%, where appropriate, relative to the total weight of the composition.

I. Film-Forming Agent that is Dispersible in an Aqueous Phase of the Composition

According to another embodiment, the film-forming polymer may be chosen from aqueous dispersions of polymer particles.

The aqueous dispersion comprising one or more film-forming polymers may be prepared by a person skilled in the art on the basis of his general knowledge, in particular by emulsion polymerization or by dispersion of the preformed polymer.

Among the film-forming polymers which may be used in the composition according to the present invention, mention may be made of synthetic polymers, of polycondensate type or of free-radical type, polymers of natural origin and mixtures thereof.

1) Polycondensates

Among the polycondensates, mention may also be made of anionic, cationic, nonionic or amphoteric polyurethanes, polyurethane-acrylics, polyurethane-polyvinylpyrrolidones, polyester-polyurethanes, polyether-polyurethanes, polyureas, polyurea/polyurethanes, and mixtures thereof.

The polyurethanes may be, for example, an aliphatic, cycloaliphatic or aromatic polyurethane, polyurea/polyurethane or polyurea copolymer, containing, alone or as a mixture:
at least one block of linear or branched aliphatic and/or cycloaliphatic and/or aromatic polyester origin, and/or
at least one block of aliphatic and/or cycloaliphatic and/or aromatic polyether origin, and/or
at least one substituted or unsubstituted, branched or unbranched silicone block, for example polydimethylsiloxane or polymethylphenylsiloxane, and/or
at least one block comprising fluoro groups.

The polyurethanes as defined in the invention may also be obtained from branched or unbranched polyesters or from alkyds containing mobile hydrogens, which are modified by means of a polyaddition with a diisocyanate and a difunctional organic co-reactive compound (for example dihydro, diamino or hydroxyamino), also containing either a carboxylic acid or carboxylate group, or a sulfonic acid or sulfonate group, or alternatively a neutralizable tertiary amine group or a quaternary ammonium group.

Mention may also be made of polyesters, polyesteramides, fatty-chain polyesters, polyamides and epoxyester resins.

The polyesters may be obtained, in a known manner, by polycondensation of aliphatic or aromatic diacids with aliphatic or aromatic diols or with polyols. Succinic acid, glutaric acid, adipic acid, pimelic acid, suberic acid or sebacic acid may be used as aliphatic diacids. Terephthalic acid or isophthalic acid, or alternatively a derivative such as phthalic anhydride, may be used as aromatic diacids. Ethylene glycol, propylene glycol, diethylene glycol, neopentyl glycol, cyclohexanedimethanol and 4,4-N-(1-methyl-propylidene)bisphenol may be used as aliphatic diols. Glycerol, pentaerythritol, sorbitol and trimethylolpropane may be used as polyols.

The polyesteramides may be obtained in a similar manner to the polyesters, by polycondensation of diacids with diamines or amino alcohols. Ethylenediamine, hexa-methylenediamine or meta- or para-phenylenediamine may be used as diamine. Monoethanolamine may be used as amino alcohol.

As monomer bearing an anionic group which may be used during the polycondensation, mention may be made, for example, of dimethylolpropionic acid, trimellitic acid or a derivative such as trimellitic anhydride, the sodium salt of pentanediol-3-sulfonic acid and the sodium salt of 5-sulfo-1,3-benzenedicarboxylic acid. The fatty-chain polyesters may be obtained using fatty-chain diols during the polycondensation. The epoxy ester resins may be obtained by polycondensation of fatty acids with a condensate having α,ω-diepoxy ends.

The free-radical polymers may in particular be acrylic and/or vinyl polymers or copolymers. Anionic radical polymers are preferred. As monomer bearing an anionic group which may be used during the free-radical polymerization, mention may be made of acrylic acid, methacrylic acid, crotonic acid, maleic anhydride or 2-acrylamido-2-methylpropanesulfonic acid.

The acrylic polymers may result from the copolymerization of monomers chosen from the esters and/or amides of acrylic acid or of methacrylic acid. As examples of monomers of ester type, mention may be made of methyl methacrylate, ethyl methacrylate, butyl methacrylate, isobutyl methacrylate, 2-ethylhexyl methacrylate and lauryl methacrylate. As examples of monomers of amide type, mention may be made of N-t-butylacrylamide and N-t-octylacrylamide.

Acrylic polymers obtained by copolymerization of ethylenically unsaturated monomers containing hydrophilic groups, preferably of nonionic nature, such as hydroxyethyl acrylate, 2-hydroxypropyl acrylate, hydroxyethyl methacrylate and 2-hydroxypropyl methacrylate, are used in particular.

The vinyl polymers may result from the homopolymerization or copolymerization of monomers chosen from vinyl esters, styrene or butadiene. As examples of vinyl esters, mention may be made of vinyl acetate, vinyl neodecanoate, vinyl pivalate, vinyl benzoate and vinyl t-butylbenzoate.

Acrylic/silicone copolymers or nitrocellulose/acrylic copolymers may also be used.

2) Polymer of Free-Radical Type

Mention may also be made of the polymers resulting from the free-radical polymerization of one or more free-radical monomers inside and/or partially at the surface of preexisting particles of at least one polymer chosen from the group consisting of polyurethanes, polyureas, polyesters, polyesteramides and/or alkyds. These polymers are generally referred to as “hybrid polymers”.

When an aqueous dispersion of polymer particles is used, the solids content of the said aqueous dispersion may be from about 3% to 60% and preferably from 10% to 50% by weight.

The size of the polymer particles in aqueous dispersion may be between 10 and 500 nm and is preferably between 20 and 150 nm, allowing the production of a film of noteworthy gloss. However, particle sizes ranging up to 1 micron may be used.

Aqueous dispersions of film-forming polymers that may be used include the acrylic dispersions sold under the names Neocryl XK-90®, Neocryl A-1070®, Neocryl A-1090®, Neocryl BT-62®, Neocryl A-1079® and Neocryl A-523® by the company Avecia-Neoresins, Dow Latex 432® by the company Dow Chemical, Daitosol 5000 AD® or Daitosol 5000 SJ by the company Daito Kasey Kogyo; Syntran 5760 by the company Interpolymer or the aqueous dispersions of polyurethane sold under the names Neorez R-981® and Neorez R-974® by the company Avecia-Neoresins, Avalure UR-405®, Avalure UR-410®, Avalure UR-425®, Avalure UR-450®, Sancure 875®, Sancure 861®, Sancure 878® and Sancure 2060® by the company Goodrich, Impranil 85® by the company Bayer and Aquamere H-1511® by the company Hydromer; the sulfopolyesters sold under the brand name Eastman AQ® by the company Eastman Chemical Products, vinyl dispersions, for instance Mexomer PAM, aqueous dispersions of polyvinyl acetate, for instance Vinybran® from the company Nisshin Chemical, or those sold by the company Union Carbide, aqueous dispersions of terpolymer of vinylpyrrolidone, dimethylaminopropylmethacrylamide and lauryldimethylpropylmethacrylamidoammonium chloride, such as Styleze W from ISP, aqueous dispersions of polyurethane/poly-acrylic hybrid polymers, such as those sold under the references Hybridur® by the company Air Products or Duromer® from National Starch, dispersions of core/shell type: for example those sold by the company Atofina under the reference Kynar (core: fluoro-shell: acrylic) or those described in document U.S. Pat. No. 5,188,899 (core: silica-shell: silicone), and mixtures thereof.

The film-forming polymer may be a water-soluble polymer. The water-soluble polymer is thus dissolved in the aqueous phase of the composition.

Among the water-soluble film-forming polymers that may be mentioned are the following cationic polymers:

1) acrylic polymers or copolymers, such as polyacrylates or polymethacrylates; the copolymers of the family (1) may also contain one or more units derived from comonomers that may be chosen from the family of acrylamides, methacrylamides, diacetoneacrylamides, acrylamides and methacrylamides substituted on the nitrogen with lower alkyls, acrylic or methacrylic acids or esters thereof, vinyllactams such as vinylpyrrolidone or vinylcaprolactam, or vinyl esters.

Thus, among these copolymers of the family (1), mention may be made of:
copolymers of acrylamide and of dimethylaminoethyl methacrylate, quaternized with dimethyl sulfate or with a dimethyl halide, such as the product sold under the name Hercofloc by the company Hercules,
the copolymer of acrylamide and of methacryloyloxy-ethyltrimethylammonium chloride described, for example, in patent application EP-A-080 976 and sold under the name Bina Quat P 100 by the company Ciba Geigy,
the copolymer of acrylamide and of methacryloyloxy-ethyltrimethylammonium methosulfate sold under the name Reten by the company Hercules,
quaternized or non-quaternized copolymers of vinyl-pyrrolidone/dialkylaminoalkyl acrylate or methacrylate, such as the products sold under the name “Gafquat” by the company ISP, for instance “Gafquat 734” or “Gafquat 755”, or alternatively the products denoted as “Copolymer 845, 958 and 937”. These polymers are described in detail in French patents 2 077 143 and 2 393 573,
terpolymers of dimethylaminoethyl methacrylate/vinylcaprolactam/vinylpyrrolidone, such as the product sold under the name Gaffix VC 713 by the company ISP, and
the quaternized copolymer of vinylpyrrolidone/-dimethylaminopropylmethacrylamide, such as the product sold under the name “Gafquat HS100” by the company ISP.

2) the quaternized polysaccharides described more particularly in U.S. Pat. No. 3,589,578 and U.S. Pat. No. 4,031,307, such as guar gums containing trialkylammonium cationic groups. Such products are sold in particular under the trade names Jaguar C138, Jaguar C15 and Jaguar C17 by the company Meyhall.

3) quaternary copolymers of vinylpyrrolidone and of vinylimidazole;

4) chitosans or salts thereof;

5) cationic cellulose derivatives such as copolymers of cellulose or of cellulose derivatives grafted with a water-soluble monomer comprising a quaternary ammonium, and described in particular in patent U.S. Pat. No. 4,131,576, such as hydroalkylcelluloses, for instance hydroxymethyl-, hydroxyethyl- or hydroxypropylcelluloses grafted in particular with a methacryloyloxyethyltrimethylammonium, methacrylamidopropyltrimethylammonium or dimethyldiallylammonium salt. The products sold corresponding to this definition are, more particularly, the products sold under the name “Celquat L 200” and “Celquat H 100” by the company National Starch.

Among the film-forming water-soluble polymers that may be mentioned are the following amphoteric polymers:

1) polymers resulting from the copolymerization of a monomer derived from a vinyl compound bearing a carboxylic group such as, more particularly, acrylic acid, methacrylic acid, maleic acid, α-chloroacrylic acid, and a basic monomer derived from a substituted vinyl compound containing at least one basic atom, such as, more particularly, a dialkylaminoalkyl methacrylate and acrylate, and a dialkylaminoalkylmethacrylamide and -acrylamide. Such compounds are described in patent U.S. Pat. No. 3,836,537.

2) polymers comprising units derived from:
a) at least one monomer chosen from acrylamides and methacrylamides substituted on the nitrogen with an alkyl radical,
b) at least one acidic comonomer containing one or more reactive carboxylic groups, and
c) at least one basic comonomer such as esters containing primary, secondary, tertiary and quaternary amine substituents of acrylic and methacrylic acids and the product of quaternization of dimethylaminoethyl methacrylate with dimethyl or diethyl sulfate.
d) crosslinked alkylpolyaminoamides totally or partially derived from polyaminoamides.

3) polymers comprising zwitterionic units.

4) chitosan-based polymers.

5) polymers derived from the N-carboxyalkylation of chitosan, such as N-carboxymethylchitosan or N-carboxybutylchitosan sold under the name “Evalsan” by the company Jan Dekker.

6) (C₁-C₅)alkyl vinyl ether/maleic anhydride copolymers, partially modified by a semi-amidation with an N,N-dialkylaminoalkylamine, such as N,N-dimethyl-aminopropylamine or by a semi-esterification with an N,N-dialkanolamine. These copolymers may also comprise other vinyl comonomers, such as vinylcaprolactam.

The water-soluble film-forming polymers are preferably chosen from the group consisting of:
proteins, for instance proteins of plant origin such as wheat proteins and soybean proteins; proteins of animal origin such as keratin, for example keratin hydrolysates and sulfonic keratins;
anionic, cationic, amphoteric or nonionic chitin or chitosan polymers;
polymers of cellulose such as hydroxyethylcellulose, hydroxypropylcellulose, methylcellulose, ethylhydroxyethylcellulose and carboxymethylcellulose, and quaternized cellulose derivatives;
acrylic polymers or copolymers, such as polyacrylates or polymethacrylates;
vinyl polymers, for instance polyvinylpyrrolidones, copolymers of methyl vinyl ether and of maleic anhydride, the copolymer of vinyl acetate and of crotonic acid, copolymers of vinylpyrrolidone and of vinyl acetate;
copolymers of vinylpyrrolidone and of caprolactam; polyvinyl alcohols;
polymers of natural origin, which are optionally modified, such as:
   gum arabic, guar gum, xanthan derivatives, karaya gum;
   alginates and carrageenans;
   glycosaminoglycans, hyaluronic acid and derivatives thereof;
   shellac resin, sandarac gum, dammar resins, elemi gums and copal resins;
   deoxyribonucleic acid;
   mucopolysaccharides such as hyaluronic acid and chondroitin sulfate,
      and mixtures thereof.

These polymers will be used in particular if a more or less appreciable removal of the film by water is desired.

In order to improve the film-forming nature of an oily or aqueous polymer, it is possible to add to the polymer system a coalescer, which will be chosen from the known coalescers.

II. Silicone-Based Film-Forming Polymer

1) Polymer with a Grafted Non-Silicone Organic Backbone

These polymers may be liposoluble, lipodispersible, water-soluble or dispersible in aqueous medium, where appropriate.

The polymers containing a non-silicone organic backbone grafted with monomers containing a polysiloxane consist of an organic main chain formed from organic monomers not comprising silicone, onto which is grafted, within the said chain and also optionally on at least one of its ends, at least one polysiloxane macromer.

In the text hereinbelow, in accordance with what is generally accepted, the expression “polysiloxane macromer” is understood to refer to any monomer containing a polysiloxane-type polymer chain in its structure.

The non-silicone organic monomers constituting the main chain of the grafted silicone polymer can be chosen from free-radical-polymerizable monomers containing ethylenic unsaturation, polycondensation-polymerizable monomers, such as those forming polyamides, polyesters or polyurethanes, and ring-opening monomers, such as those of the oxazoline or caprolactone type.

The polymers containing a non-silicone organic backbone grafted with monomers containing a polysiloxane, in accordance with the present invention, can be obtained according to any means known to those skilled in the art, in particular by reaction between (i) a starting polysiloxane macromer which is correctly functionalized on the polysiloxane chain and (ii) one or more non-silicone organic compounds, themselves correctly functionalized with a function which is capable of reacting with the functional group(s) borne by the said silicone, forming a covalent bond; a classic example of such a reaction is the free-radical reaction between a vinyl group borne on one of the ends of the silicone with a double bond of a monomer containing ethylenic unsaturation in the main chain.

The polymers containing a non-silicone organic backbone grafted with monomers containing a polysiloxane, in accordance with the invention, are more preferably chosen from those described in U.S. Pat. No. 4,693,935, U.S. Pat. No. 4,728,571 and U.S. Pat. No. 4,972,037 and patent applications EP-A-0 412 704, EP-A-O-412 707, EP-A-0 640 105 and WO 95/00578. These are copolymers obtained by free-radical polymerization starting with monomers containing ethylenic unsaturation and monomers having a terminal vinyl group, or alternatively copolymers obtained by reaction of a polyolefin comprising functionalized groups and a polysiloxane macromer having a terminal function which is reactive with the said functionalized groups.

One particular family of grafted silicone polymers which is suitable for carrying out the present invention consists of grafted silicone polymers comprising:

a) from 0 to 98% by weight of at least one free-radical-polymerizable lipophilic monomer (A) of low lipophilic polarity containing ethylenic unsaturation;

b) from 0 to 98% by weight of at least one polar hydrophilic monomer (B) containing ethylenic unsaturation, which is copolymerizable with the monomer(s) of the type (A);

c) from 0.01% to 50% by weight of at least one polysiloxane macromer (C) of general formula (XXVII):
X(Y)ₙSi(R)₃₋ₘZₘ (XXVII)
in which:
X denotes a vinyl group which is copolymerizable with the monomers (A) and (B);
Y denotes a divalent bonding group;
R denotes hydrogen, C₁-C₆ alkyl or alkoxy, or C₆-C₁₂ aryl;
Z denotes a monovalent polysiloxane unit with a number-average molecular weight of at least 500;
n is 0 or 1 and m is an integer ranging from 1 to 3; the percentages being calculated relative to the total weight of the monomers (A), (B) and (C).

These polymers have a number-average molecular weight ranging from 10 000 to 2 000 000 and preferably a glass transition temperature Tg or a crystal melting temperature Tm of at least −20° C.

As examples of lipophilic monomers (A), mention may be made of acrylic or methacrylic acid esters of C₁-C₁₈ alcohols; methacrylic acid esters of C₁₂-C₃₀ alcohols, styrene; polystyrene macromers; vinyl acetate; vinyl propionate; α-methylstyrene; tert-butylstyrene; butadiene; cyclohexadiene; ethylene; propylene; vinyltoluene; acrylic or methacrylic acid esters of 1,1-dihydroperfluoroalkanols or of homologues thereof; acrylic or methacrylic acid esters of ω-hydrofluoroalkanols; acrylic or methacrylic acid esters of fluoroalkylsulfonamido alcohols; acrylic or methacrylic acid esters of fluoroalkyl alcohols; acrylic or methacrylic acid esters of fluoroether alcohols; or mixtures thereof. The preferred monomers (A) are chosen from the group consisting of n-butyl methacrylate, isobutyl methacrylate, tert-butyl acrylate, tert-butyl methacrylate, 2-ethylhexyl methacrylate, methyl methacrylate, 2-(N-methylperfluorooctanesulfonamido)ethyl acrylate and 2-(N-butylperfluorooctanesulfonamido)ethyl acrylate, or mixtures thereof.

As examples of polar monomers (B), mention may be made of acrylic acid, methacrylic acid, N,N-dimethylacrylamide, dimethylaminoethyl methacrylate, quaternized dimethylaminoethyl methacrylate, (meth)acrylamide, N-t-butylacrylamide, maleic acid, maleic anhydride and hemiesters thereof, hydroxyalkyl (meth)acrylates, diallyldimethylammonium chloride, vinyl-pyrrolidone, vinyl ethers, maleimides, vinylpyridine, vinylimidazole, heterocyclic vinyl polar compounds, styrene sulfonate, allyl alcohol, vinyl alcohol and vinylcaprolactam, or mixtures thereof. The monomers (B) are preferably chosen from the group consisting of acrylic acid, N,N-dimethylacrylamide, dimethylaminoethyl methacrylate, quaternized dimethylaminoethyl methacrylate and vinylpyrrolidone, and mixtures thereof.

Mention is made especially of the product KP 561 or KP 562 sold by Shin-Etsu such that the monomer (A) is chosen from esters of a C₁₈-C₂₂ alcohol and of methacrylic acid.

The polysiloxane macromers (C) of formula (XXVII) are preferably chosen from those corresponding to the general formula (XXVIII) below: 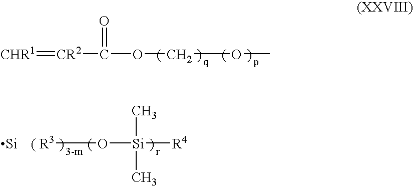
in which:
R¹ is hydrogen or —COOH (preferably hydrogen);
R² is hydrogen, methyl or —CH₂COOH (preferably methyl);
R³ is C₁-C₆ alkyl, alkoxy, or alkylamino, C₆-C₁₂ aryl or hydroxyl (preferably methyl);
R⁴ is C₁-C₆ alkyl, alkoxy or alkylamino, C₆-C₁₂ aryl or hydroxyl (preferably methyl);
q is an integer ranging from 2 to 6 (preferably 3);
p is 0 or 1;
r is an integer ranging from 5 to 700;
m is an integer ranging from 1 to 3 (preferably 1).

The polysiloxane macromers of formula (XXIX): 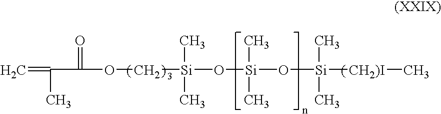
with n being a number ranging from 5 to 700 and I being an integer between 0 and 3, are preferably used.

One embodiment of the invention consists in using a copolymer which may be obtained by free-radical polymerization starting with the monomer mixture consisting of:

a) 60% by weight of tert-butyl acrylate;

b) 20% by weight of acrylic acid;

c) 20% by weight of silicone macromer of formula (XXX): 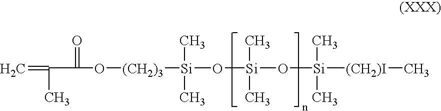
n being a number ranging from 5 to 700 and I being an integer between 0 and 3; the weight percentages being calculated relative to the total weight of the monomers.

Another particular embodiment of the invention consists in using a copolymer which may be obtained by free-radical polymerization starting with the monomer mixture consisting of:

a) 80% by weight of tert-butyl acrylate;

b) 20% by weight of silicone macromer of formula (XXXI): 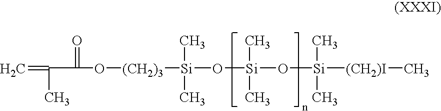
with n being a number ranging from 5 to 700 and I being an integer between 0 and 3; the weight percentages being calculated relative to the total weight of the monomers.

Another particular family of grafted silicone polymers with a non-silicone organic backbone that is suitable for carrying out the present invention consists of grafted silicone copolymers which may be obtained by reactive extrusion-moulding of a polysiloxane macromer with a reactive terminal function on a polymer of the polyolefin type comprising reactive groups capable of reacting with the terminal function of the polysiloxane macromer to form a covalent bond for grafting the silicone onto the main chain of the polyolefin. These polymers are described, along with a process for their preparation, in patent application WO 95/00578.

The reactive polyolefins are preferably chosen from polyethylenes and polymers of ethylene-derived monomers such as propylene, styrene, alkylstyrene, butylene, butadiene, (meth)acrylates, vinyl esters or equivalents, comprising reactive functions capable of reacting with the terminal function of the polysiloxane macromer. They are chosen more particularly from copolymers of ethylene or of ethylene derivatives and of monomers chosen from those comprising a carboxylic function such as (meth)acrylic acid; those comprising an acid anhydride function such as maleic anhydride; those comprising an acid chloride function such as (meth)acryloyl chloride; those comprising an ester function such as (meth)acrylic acid esters; and those comprising an isocyanate function.

The silicone macromers are preferably chosen from polysiloxanes comprising a functionalized group, at the end of the polysiloxane chain or close to the end of the said chain, chosen from the group consisting of alcohols, thiols, epoxy groups and primary and secondary amines, and more particularly from those corresponding to the general formula (XXXII):
T-(CH₂)₆—Si—[—(OSiR⁵R⁶)ₜ—R⁷]_{y} (XXXII)
in which T is chosen from the group consisting of NH₂, NHRN and an epoxy, OH, or SH function; R⁵, R⁶, R⁷ and RN independently denote a C₁-C₆ alkyl, phenyl, benzyl, or C₆-C₁₂ alkylphenyl or hydrogen; s is a number ranging from 2 to 100; t is a number ranging from 0 to 1000 and y is a number ranging from 1 to 3. They have a number-average molecular weight preferably ranging from 5000 to 300 000, more preferably from 8000 to 200 000 and more particularly from 9000 to 40 000.

According to one preferred embodiment, the film-forming polymer may be purchased from the Minnesota Mining and Manufacturing Company under the trade name “Silicone Plus” polymers. For example, poly(isobutyl methacrylate-co-methyl FOSEA)-g-poly(dimethylsiloxane) is sold under the trade name SA 70-5 IBMMF.

2) Polymer with a Silicone-Based Backbone

The said grafted silicone polymer(s) containing a polysiloxane backbone grafted with non-silicone organic monomers comprising a silicone (or polysiloxane (/SiO—)ₙ) main chain onto which is grafted, within the said chain and also optionally on at least one of its ends, at least one organic group not comprising silicone.

The polymers containing a polysiloxane backbone grafted with non-silicone organic monomers, according to the invention, can be existing commercial products or alternatively can be obtained by any means known to those skilled in the art, in particular by reaction between (i) a starting silicone which is correctly functionalized on one or more of these silicon atoms, and (ii) a non-silicone organic compound which is itself correctly functionalized with a function which is capable of reacting with the functional group(s) borne by the said silicone, forming a covalent bond; a classic example of such a reaction is the hydrosilylation reaction between /Si—H groups and vinyl groups CH₂═CH—, or alternatively the reaction between thio functional groups —SH with these same vinyl groups.

Examples of polymers containing a polysiloxane backbone grafted with non-silicone organic monomers that are suitable for carrying out the present invention, and also their specific mode of preparation, are described in particular in patent applications EP-A-0 582 152, WO 93/23009 and WO 95/03776, the teachings of which are included in their entirety in the present description by way of non-limiting references.

According to a particularly preferred embodiment of the present invention, the silicone polymer containing a polysiloxane backbone grafted with non-silicone organic monomers which is used, comprises the result of a free-radical copolymerization between, on the one hand, at least one non-silicone anionic organic monomer containing ethylenic unsaturation and/or a non-silicone hydrophobic organic monomer containing ethylenic unsaturation, and, on the other hand, a silicone containing in its chain at least one, and preferably several, functional group(s) capable of reacting with the said ethylenic unsaturations of the said non-silicone monomers, forming a covalent bond, in particular thio functional groups.

According to the present invention, the said anionic monomers containing ethylenic unsaturation are preferably chosen, alone or as mixtures, from linear or branched, unsaturated carboxylic acids, optionally partially or totally neutralized in the form of a salt, it being possible for this or these unsaturated carboxylic acid(s) to be, more particularly, acrylic acid, methacrylic acid, maleic acid, itaconic acid, fumaric acid and crotonic acid. The suitable salts are, in particular, alkali metal salts, alkaline-earth metal salts and ammonium salts. It will likewise be noted that, in the final grafted silicone polymer, the organic group of anionic nature which comprises the result of the free-radical (homo)polymerization of at least one anionic monomer of unsaturated carboxylic acid type can, after reaction, be post-neutralized with a base (sodium hydroxide, aqueous ammonia, etc.) in order to place it in the form of a salt.

According to the present invention, the hydrophobic monomers containing ethylenic unsaturation are preferably chosen, alone or as a mixture, from acrylic acid esters of alkanols and/or methacrylic acid esters of alkanols. The alkanols are preferably of C₁-C₃₀ and more particularly of C₁-C₂₂. The preferred monomers are chosen from the group consisting of isooctyl (meth)acrylate, isononyl (meth)acrylate, 2-ethylhexyl (meth)acrylate, lauryl (meth)acrylate, isopentyl (meth)acrylate, n-butyl (meth)acrylate, isobutyl (meth)acrylate, methyl (meth)acrylate, tert-butyl (meth)acrylate, tridecyl (meth)acrylate and stearyl (meth)acrylate, or mixtures thereof.

One family of silicone polymers containing a polysiloxane backbone grafted with non-silicone organic monomers that is particularly suitable for carrying out the present invention consists of silicone polymers comprising in their structure the unit of formula (XXXIII) below: 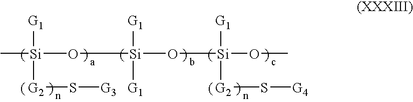
in which the radicals G₁, which may be identical or different, represent hydrogen, a C₁-C₁₀ alkyl radical or a phenyl radical; the radicals G₂, which may be identical or different, represent a C₁-C₁₀ alkylene group; G₃ represents a polymer residue resulting from the (homo)polymerization of at least one anionic monomer containing ethylenic unsaturation; G₄ represents a polymer residue resulting from the (homo)polymerization of at least one hydrophobic monomer containing ethylenic unsaturation; m and n are equal to 0 or 1; a is an integer ranging from 0 to 50; b is an integer which may be between 10 and 350, c is an integer ranging from 0 to 50; with the proviso that one of the parameters a and c is other than 0.

Preferably, the unit of formula (XXXIII) of the above text has at least one, and even more preferably all, of the following characteristics:
the radicals G₁ denote an alkyl radical, preferably a methyl radical;
n is not zero, and the radicals G₂ represent a divalent C₁-C₃ radical, preferably a propylene radical;
G₃ represents a polymer radical resulting from the (homo)polymerization of at least one monomer of the carboxylic acid type containing ethylenic unsaturation, preferably acrylic acid and/or methacrylic acid;
G₄ represents a polymer radical resulting from the (homo)polymerization of at least one monomer of the (C₁-C₁₀) alkyl (meth)acrylate type, preferably isobutyl or methyl (meth)acrylate.

Examples of silicone polymers corresponding to formula (XXXIII) are, in particular, polydimethylsiloxanes (PDMSs) onto which are grafted, via a thiopropylene-type secondary bond, mixed polymer units of the poly(meth)acrylic acid type and of the polyalkyl (meth)acrylate type.

Other examples of silicone polymers corresponding to formula (XXXIII) are, in particular, polydimethylsiloxanes (PDMSs) onto which are grafted, via a thiopropylene-type secondary bond, polymer units of the polyisobutyl (meth)acrylate type.

Such polymers comprise polymers comprising at least one group of formula (XXXIV): 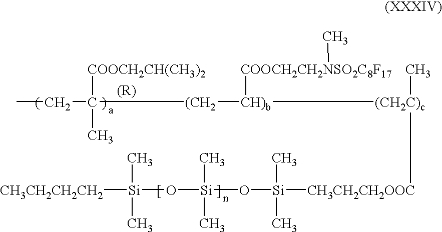
in which
a, b and c, which may be identical or different, are each a number ranging from 1 to 100 000; and the end groups, which may be identical or different, are each chosen from linear C₁-C₂₀ alkyl groups, C₃-C₂₀ branched-chain alkyl groups, C₃-C₂₀ aryl groups, linear C₁-C₂₀ alkoxy groups and branched C₃-C₂₀ alkoxy groups.

Such polymers are disclosed in U.S. Pat. Nos. 4,972,037, 5,061,481, 5,209,924, 5,849,275, 6,033,650 and WO 93/23446 and WO 95/06078.

Another family of silicone polymers having a polysiloxane backbone grafted with non-silicone organic monomers, which is particularly suitable for performing the present invention, consists of silicone polymers comprising in their structure the unit of formula (XXXV) below: 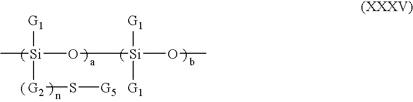
in which the radicals G₁ and G₂ have the same meaning as above; G₅ represents a polymer residue resulting from the (homo)polymerization of at least one ethylenically unsaturated hydrophobic monomer or from the copolymerization of at least one ethylenically unsaturated anionic monomer and of at least one ethylenically-unsaturated hydrophobic monomer; n is equal to 0 or 1; a is an integer ranging from 0 to 50; b is an integer that may be between 10 and 350; on condition that a is other than 0.

The unit of formula (XXXV) in the above text preferably has at least one, and even more preferably all, of the following characteristics:
the radicals G₁ denote an alkyl radical, preferably a methyl radical;
n is not zero, and the radicals G₂ represent a C₁-C₃ divalent radical, preferably a propylene radical.

The number-average molar mass of the silicone polymers with a polysiloxane backbone grafted with non-silicone organic monomers of the invention preferably ranges from about 10 000 to 1 000 000 and even more preferably from about 10 000 to 100 000.

According to one particular embodiment, a film-forming silicone polymer may be a copolymer comprising carboxylate groups and polydimethylsiloxane groups.

In the present patent application, the expression “copolymer comprising carboxylate groups and polydimethylsiloxane groups” means a copolymer obtained from (a) one or more carboxylic (acid or ester) monomers, and (b) one or more polydimethylsiloxane (PDMS) chains.

In the present patent application, the term “carboxylic monomer” means both carboxylic acid monomers and carboxylic acid ester monomers. Thus, the monomer (a) may be chosen, for example, from acrylic acid, methacrylic acid, maleic acid, fumaric acid, itaconic acid, crotonic acid, esters thereof and mixtures of these monomers. Esters that may be mentioned include the following monomers: acrylate, methacrylate, maleate, fumarate, itaconate and/or crotonate. The monomers in ester form are more particularly chosen from linear or branched, preferably C₁-C₂₄ and better still C₁-C₂₂ alkyl acrylates and methacrylates, the alkyl radical preferably being chosen from methyl, ethyl, stearyl, butyl and 2-ethylhexyl radicals, and mixtures thereof.

The copolymer may comprise as carboxylate groups at least one group chosen from acrylic acid and methacrylic acid, and methyl, ethyl, stearyl, butyl or 2-ethylhexyl acrylate or methacrylate, and mixtures thereof.

The term “polydimethylsiloxanes” (also known as organopolysiloxanes and abbreviated as PDMS) denotes, in accordance with what is generally accepted, any organosilicon polymer or oligomer of linear structure, of variable molecular weight, obtained by polymerization and/or polycondensation of suitably functionalized silanes, and consisting essentially of a repetition of main units in which the silicon atoms are linked together via oxygen atoms (siloxane bond ≡Si—O—Si≡), comprising trimethyl radicals directly linked via a carbon atom to the said silicon atoms. The PDMS chains that may be used to obtain the copolymer comprise at least one polymerizable radical group, preferably located on at least one of the ends of the chain, i.e. the PDMS may contain, for example, a polymerizable radical group on the two ends of the chain or one polymerizable radical group on one end of the chain and one trimethylsilyl end group on the other end of the chain. The polymerizable radical group may especially be an acrylic or methacrylic group, in particular a group CH₂═CR₁—CO—O—R₂, in which R₁ represents a hydrogen or a methyl group and R₂ represents —CH₂—, —(CH₂)ₙ— with n=3, 5, 8 or 10, —CH₂—CH(CH₃)—CH₂—, —CH₂—CH₂—O—CH₂—CH₂—, —CH₂—CH₂—O—CH₂—CH₂—CH(CH₃)—CH₂—, —CH₂—CH₂—O—CH₂CH₂—O—CH₂—CH₂—CH₂—.

The copolymers used are generally obtained according to the usual methods of polymerization and grafting, for example by free-radical polymerization (A) of a PDMS comprising at least one polymerizable radical group (for example on one of the ends of the chain or on both ends) and (B) of at least one carboxylic monomer, as described, for example, in documents U.S. Pat. No. 5,061,481 and U.S. Pat. No. 5,219,560.

The copolymers obtained generally have a molecular weight ranging from about 3000 to 200 000 and preferably from about 5000 to 100 000.

The copolymer may be in its native form or in dispersed form in a solvent such as lower alcohols containing from 2 to 8 carbon atoms, for instance isopropyl alcohol, or oils, for instance volatile silicone oils (for example cyclopentasiloxane).

As copolymers that may be used, mention may be made, for example, of copolymers of acrylic acid and of stearyl acrylate containing polydimethylsiloxane grafts, copolymers of stearyl methacrylate containing polydimethylsiloxane grafts, copolymers of acrylic acid and of stearyl methacrylate containing polydimethylsiloxane grafts, copolymers of methyl methacrylate, butyl methacrylate, 2-ethylhexylacrylate and stearyl methacrylate containing polydimethylsiloxane grafts. As copolymer that may be used, mention may be made in particular of the copolymers sold by the company Shin-Etsu under the names KP-561 (CTFA name: acrylates/dimethicone), KP-541 in which the copolymer is dispersed at 60% by weight in isopropyl alcohol (CTFA name: acrylates/dimethicone and isopropyl alcohol), KP-545 in which the copolymer is dispersed at 30% in cyclopentasiloxane (CTFA name: acrylates/dimethicone and cyclopentasiloxane). According to one preferred embodiment of the invention, KP561 is preferably used; this copolymer is not dispersed in a solvent, but is in waxy form, its melting point being about 30° C.

More generally, the total amount of polymer should be an amount that is sufficient to form on the skin and/or the lips a cohesive film capable of following the movements of the skin and/or the lips without becoming detached or cracking.

When the glass transition temperature of the polymer is too high for the desired use, a plasticizer may be combined therewith so as to lower this temperature of the mixture used. The plasticizer may be chosen from the plasticizers usually used in the field of application, and especially from compounds that may be solvents for the polymer.

Needless to say, this list of polymers is not exhaustive.

Fillers

The composition may comprise fillers, in particular colorless fillers, in the medium.

The term “fillers” denotes particles of any form that are insoluble in the medium of the composition, irrespective of the temperature of which the composition is manufactured. These fillers may serve especially to modify the rheology or the texture of the composition.

Examples of fillers that may be mentioned, inter alia, include talc, mica, silica, kaolin, and polyamide powders (for example Nylon® powder or Orgasol® powder from Atochem).

In some embodiments of the invention, the fillers can be white or colorless in the medium. Colorless fillers are preferably used in the medium rather than white fillers in the medium.

Examples of colorless fillers in the medium that can be mentioned amongst others are mica, and thermoplastic material powders, polyamide powders (e.g. Nylon® or Orgasol from Atochem), polyethylene terephthalate (PET), polyethylene (PE), polypropylene (PP), polyvinyl chloride (PVC), polymethyl methacrylate (PMMA), polycarbonate (PC) powders.

Examples of white fillers in the medium that can be mentioned amongst others are talc titanium dioxide, barium sulfate, kaolin, silica, and magnesium sulfate.

The filler content will be chosen so as not to excessively hamper the interference phenomenon responsible for the red overbrightness points.

Active Agents and Other Compounds

The cosmetic composition may also contain one or more cosmetic, dermatological, hygiene or pharmaceutical active agents.

As cosmetic, dermatological, hygiene or pharmaceutical active agents that may be used in the compositions of the invention, mention may be made of moisturizers (polyols, for instance glycerol), vitamins (C, A, E, F, B or PP), essential fatty acids, essential oils, ceramides, sphingolipids, sunscreens that are liposoluble or in the form of nanoparticles, and specific skin-treating active agents (protective agents, antibacterial agents, anti-wrinkle agents, etc.). These active agents may be used, for example, in concentrations of from 0.001% to 15% relative to the total weight of the composition.

The cosmetic composition may also contain ingredients commonly used in cosmetics, for instance thickeners, surfactants, trace elements, moisturizers, softeners, sequestrants, fragrances, acidifying or basifying agents, preserving agents, antioxidants and UV-screening agents, or mixtures thereof.

Depending on the intended type of application, the cosmetic composition may also comprise the constituents conventionally used in the fields under consideration, which are present in an amount that is suitable for the intended galenical form.

Other Colouring Agents

The composition may comprise one or more scattering pigments, in a proportion that makes it possible to conserve the interference phenomenon responsible for the red overbrightness points.

This or these scattering pigment(s) may thus be in a content such that the total content of solids other than the red interference pigment in the composition does not exceed 0.3%.relative to the total weight of the composition.

Various scattering pigments may be envisaged, being chosen, for example, from organic pigments or lakes selected especially from the materials below, and mixtures thereof:
cochineal carmine,
organic pigments of azo, anthraquinone, indigoid, xanthene, pyrene, quinoline, triphenylmethane or fluorane dyes,
organic lakes or insoluble sodium, potassium, calcium, barium, aluminium, zirconium, strontium or titanium salts of acidic dyes such as azo, anthraquinone, indigoid, xanthene, pyrene, quinoline, triphenylmethane or fluorane dyes, these dyes possibly comprising at least one carboxylic or sulfonic acid group.

Among the organic pigments that may especially be mentioned are those known under the following names: D&C Blue No. 4, D&C Brown No. 1, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5, FD&C Yellow No. 6.

The lake may be supported on an organic support such as rosin or aluminium benzoate, for example.

Among the organic lakes that may be mentioned in particular are those known under the following names: D&C Red No. 2 Aluminium lake, D&C Red No. 3 Aluminium lake, D&C Red No. 4 Aluminium lake, D&C Red No. 6 Aluminium lake, D&C Red No. 6 Barium lake, D&C Red No. 6 Barium/Strontium lake, D&C Red No. 6 Strontium lake, D&C Red No. 6 Potassium lake, D&C Red No. 7 Aluminium lake, D&C Red No. 7 Barium lake, D&C Red No. 7 Calcium lake, D&C Red No. 7 Calcium/Strontium lake, D&C Red No. 7 Zirconium lake, D&C Red No. 8 Sodium lake, D&C Red No. 9 Aluminium lake, D&C Red No. 9 Barium lake, D&C Red No. 9 Barium/Strontium lake, D&C Red No. 9 Zirconium lake, D&C Red No. 10 Sodium lake, D&C Red No. 19 Aluminium lake, D&C Red No. 19 Barium lake, D&C Red No. 19 Zirconium lake, D&C Red No. 21 Aluminium lake, D&C Red No. 21 Zirconium lake, D&C Red No. 22 Aluminium lake, D&C Red No. 27 Aluminium lake, D&C Red No. 27 Aluminium/Titanium/Zirconium lake, D&C Red No. 27 Barium lake, D&C Red No. 27 Calcium lake, D&C Red No. 27 Zirconium lake, D&C Red No. 28 Aluminium lake, D&C Red No. 30 lake, D&C Red No. 31 Calcium lake, D&C Red No. 33 Aluminium lake, D&C Red No. 34 Calcium lake, D&C Red No. 36 lake, D&C Red No. 40 Aluminium lake, D&C Blue No. 1 Aluminium lake, D&C Green No. 3 Aluminium lake, D&C Orange No. 4 Aluminium lake, D&C Orange No. 5 Aluminium lake, D&C Orange No. 5 Zirconium lake, D&C Orange No. 10 Aluminium lake, D&C Orange No. 17 Barium lake, D&C Yellow No. 5 Aluminium lake, D&C Yellow No. Zirconium lake, D&C Yellow No. 6 Aluminium lake, D&C Yellow No. 7 Zirconium lake, D&C Yellow No. 10 Aluminium lake, FD&C Blue No. 1 Aluminium lake, FD&C Red No. 4 Aluminium lake, FD&C Red No. 40 Aluminium lake, FD&C Yellow No. 5 Aluminium lake, FD&C Yellow No. 6 Aluminium lake.

The chemical materials corresponding to each of the organic dyestuffs mentioned above are mentioned in the publication “International Cosmetic Ingredient Dictionary and Handbook”, 1997 edition, pages 371 to 386 and 524 to 528, published by “The Cosmetic, Toiletry, and Fragrance Association”, the content of which is incorporated into the present patent application by reference.

The scattering pigment may be a composite pigment, comprising a core at least partially coated with a shell. Such a composite pigment may be composed especially of particles comprising a mineral core and at least one at least partial coating of at least one organic dyestuff. At least one binder may advantageously contribute to the fixing of the organic dyestuff to the mineral core.

The composite pigment particles may have varied forms. These particles may especially be in platelet or globular form, in particular spherical, and may be hollow or solid. The term “platelet form” denotes particles for which the ratio of the largest size to the thickness is greater than or equal to 5. A composite pigment may have, for example, a specific surface area of between 1 and 1000 m²/g, especially between 10 and 600 m²/g approximately and in particular between 20 and 400 m²/g approximately. The specific surface area is the value measured by the BET method. The mass proportion of the core may exceed 50% and may range, for example, from 50% to 70%, for example from 60% to 70%, relative to the total weight of the composite pigment.

The pigment may also be an inorganic pigment, especially a nacre or a reflective particle with a metallic tint.

The other colouring agent(s) may also be chosen from pigments with effects, especially goniochromatic pigments and scattering pigments, and dyes.

It may be a dye of plant, animal or mineral origin, in particular of plant or mineral origin and especially of plant origin. This dye may be of non-synthetic nature.

The dye may be a water-soluble or liposoluble natural dye.

As illustrations of natural water-soluble colouring agents that may be used according to the invention, mention may be made of caramel, beetroot juice, carmine, betanin (beetroot), cuprous chlorophylline, methylene blue, anthocyanins (enocyanin, black carrot, hibiscus or elder) and riboflavin.

As illustrations of natural liposoluble colouring agents that may be used, mention may be made particularly of Sudan red, β-carotene, carotenoids, lycopene, palm oil, Sudan brown, quinoline yellow, xanthophylls (capsanthin, capsorubin or lutein), and curcumin.

As other natural colouring agents that are most particularly suitable for use in the invention, mention may be made more particularly of anthocyans from flowers or from fruit or derivatives thereof, flavonoids and tannins extracted from native or fermented plants, juglone, lawsone, extracts of fermented soybean, of algae, of fungi or of microorganisms, flavylium salts that are unsubstituted in position 3, as described in patent EP 1 172 091, extracts of Gesneria fulgens, Blechum procerum or Saxifraga, and pigments that may be obtained by extraction with an organic or aqueous-organic solvent of a culture medium of micromycetes of the Monascus type.

Examples of synthetic dyes that may be mentioned include synthetic liposoluble dyes, for instance DC Red 17, DC Red 21, DC Red 27, DC Green 6, DC Yellow 11, DC Violet 2 and DC Orange 5.

Examples of synthetic water-soluble dyes that may be mentioned include FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5 and FDC Blue 1.

Reflective Particles Having a Metallic Glint

Various reflective particles having a metallic glint can be envisaged, in particular those presenting reflectivity that is high enough to create highlights with an intensity that is greater than or equal to 3000 cd·m⁻², better 4 000 cd m⁻², and for example less than or equal to 5 000 cd m⁻².

The ratio m₁/m₂ of the weight m₁ of red interference pigment over the weight m₂ of reflective particles can lie in the range 0.1 to 1.5.

Their size can lie in the range 10 μm to 500 μm, for example, preferably lying in the range 10 μm to 150 μm. The size can advantageously be greater than or equal to 40 μm.

The reflective particles can be in the form of flakes, thereby making the reflection more directional, or, in contrast, they can present a substantially spherical shape, in order to provide reflection that is more diffuse.

By way of example, the reflective particles have a metallic glint, and they advantageously include at least one electrically-conductive surface layer that is formed by at least one metal or metal oxide.

Regardless of their form, the reflective particles having a metallic glint may optionally have a multilayer structure; with a multilayer structure, they may, for example, have at least one layer preferably having uniform thickness, in particular of a reflective material, advantageously a metal compound.

When the reflective particles having a metallic glint do not have a multilayer structure, they may, for example, be composed of at least one metal compound, e.g. a metal oxide, in particular an iron oxide obtained by synthesis.

When the reflective particles have a multilayer structure they may, for example, comprise a natural or synthetic substrate, in particular a synthetic substrate which is at least partially coated with at least one layer of a reflective material, in particular at least one layer of at least one metal compound such as a metal or an alloy. The substrate may be a single material or multiple materials, and it may be organic and/or inorganic. More particularly, the substrate may be selected from glasses, ceramics, graphite, metal oxides, aluminas, silicas, silicates, in particular aluminosilicates and borosilicates, synthetic mica, and mixtures thereof, this list not being limiting.

Examples of reflective particles comprising a mineral substrate coated with a metal layer that may be mentioned are particles comprising a substrate of borosilicate coated with silver. Glass substrate particles coated with silver in the form of flakes are sold under the trade name MICROGLASS METASHINE REFSX 2025 PS by TOYAL. Glass substrate particles coated with nickel/chromium/molybdenum alloy are sold under the trade name CRYSTAL STAR GF 550, GF 2525 by the same company.

Regardless of their form, the reflective particles having a metallic glint may also be selected from particles of synthetic substrate at least partially coated with at least one layer of at least one metal oxide selected, for example, from oxides of titanium, in particular TiO₂, of iron, in particular Fe₂O₃, of tin, or of chromium, barium sulfate, and the following materials: MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅, MOS₂, and their mixtures or alloys.

Examples of such particles that may be mentioned are particles comprising a substrate of synthetic mica coated with titanium dioxide, or glass particles coated either with brown iron oxide, titanium oxide, tin oxide, or one of their mixtures such as those sold under the trade name REFLECKS® by ENGELHARD.

Other examples of reflective particles having a metallic glint, presenting a metal compound at their surface or including at least one coated metal compound, and that may be mentioned are the particles proposed under the trade name METASHINE® ME 2040 PS, METASHINE® MC5090 PS, or METASHINE® MC280GP (2523) by NIPPON SHEET GLASS, SPHERICAL SILVER POWDER® DC 100, SILVER FLAKE® JV6, or GOLD POWDER® A1570 by ENGELHARD, STARLIGHT REFLECTIONS FXM® by ENERGY STRATEGY ASSOCIATES INC. BRIGHT SILVER® 1 E 0.008X0.008 by MEADOWBROOK INVENTIONS, ULTRAMIN® (ALUMINIUM POUDRE FINE LIVING), and COSMETIC METALLIC POWDER VISIONAIRE BRIGHT SILVER SEA®, COSMETIC METALLIC POWDER VISIONAIRE NATURAL GOLD® (60314), or COSMETIC METALLIC POWDER VISIONAIRE HONEY® (60316) by ECKART.

The reflective particles having a metallic glint may reflect the visible spectrum in substantially uniform manner, e.g. as with particles that are optionally coated in a metal such as silver or aluminum, which can thus lead to a metallic glint having a non-neutral, yellow, pink, red, bronze, orange, brown, gold, and/or copper glint, depending on the kind of metal compound at the surface, for example.

The reflective particles having a metallic glint may be present in the composition in an amount in the range 0.1% to 60% by weight relative to the total weight of the first composition, specifically 1% to 30% by weight, e.g. 3% to 10% by weight.

When reflective particles have a multilayer structure with a core, the core can be in the same material as the core of the red interference pigment.

Silvery Reflective Pigments

This pigment reflects the incident light spectrum in substantially uniform manner.

Examples of silvery reflective pigments that may be mentioned are silvery reflective particles TIMICA SPARKLE 110P®, TIMICA SILKBLANC 110W®, FLAMENCO SUPERPEARL 120 C+®, TIMICA EXTRA LARGE SPARKLE 110S®, FLAMENCO PEARL 110C®, TIMICA PEARL WHITE 110 A®, TIMICA SILVER SPARKLE 5500/EP 94003®, FLAMENCO SATIN PEARL 3500® sold by ENGELHARD, silvery reflective particles NAILSYN PLATINUM 60®, XIRONA SILVER®, BIRON LF 2000® (ref 117077), TIMIRON SNOWFLAKE MP 99® (117470), LOW LUSTRE PIGMENT® (17399), TIMIRON DIAMOND CLUSTER MP 149® (17266), TIMIRON ULTRALUSTER MP 111® (117226), TIMIRON PEARL SHEEN MP 30® (17216), TIMIRON SUPER SILK MP 1005® (17203) sold by MERCK, silvery reflective particles PRESTIGE SPARKLING SILVER® (35178), PRESTIGE SPARKLING SILVER STAR® (35179) sold by ECKART, silvery reflective particles SUNSHINE FINE WHITE® (C80-3100), SUNSHINE GLITTER WHITE® (C80-3400) sold by SUN, and silvery reflective particles KTZ CLASSIC WHITE® (10-40 MICRONS), KTZ STELLAR WHITE® (20-80 MICRONS) sold by TAIZHU.

Colored Reflective Pigments

Various colored reflective pigments other than the red interference pigment can be envisaged, provided they present reflectivity that is high enough to create highlights with an intensity that is greater than or equal to 3000 cd·m⁻², better 4 000 cd m⁻², and for example less than or equal to 5 000 cd m⁻².

Their size is preferably greater than or equal to 30 μm, better 40 μm, advantageously being of the same order as the size of the red interference pigment, to within 10%, in order to obtain a pixellization effect that is more uniform. In particular, the size can lie in the range 30 μm to 80 μm, for example.

The colored reflective pigment can have a dominant wavelength that is different from the dominant wavelength of the red interference pigment, e.g. 580 nm or less, measured with the above-mentioned calorimeter, under the measurement conditions used for measuring the intensity of the highlights.

It can be advantageous for the colored reflective pigment to have a core of the same material as the red interference pigment, since that makes it possible to have highlight intensities of the same order, to within 10%.

The expression “of the same order, to within 10%” signifies that the size or the highlight intensity of the reflective pigment is in the range 0.9 to 1.1 times the size or the highlight intensity of the red interference pigment.

The surface layer of the colored reflective pigment can be of the same material as the surface layer of the red interference pigment, in particular when the core is also of the same material, the pigments thus differing by the thickness of the surface layer, for example, thereby making it possible to generate another color by the interference phenomenon.

By way of example, the proportion of colored reflective pigment lies in the range 0.1 to 10 times the proportion of the red interference pigment.

Proportions similar to within 10% make it possible to obtain a uniform effect.

The colored reflective pigments can be selected from goniochromatic nacres et interference pigments, amongst others.

The term “nacre” means colored particles of any form, which may optionally be iridescent, as produced in the shells of certain mollusks, or which are synthesized, and which exhibit a “pearlescent” coloring effect by an interference phenomenon.

Nacres may be selected from nacre pigments such as mica titanium coated with iron oxide, mica coated with bismuth oxychloride, mica titanium coated with chromium oxide, mica titanium coated with an organic colorant, in particular of the type mentioned above, and nacre pigments based on bismuth oxychloride. They may also be particles of mica on the surface of which at least two successive layers of metal oxides and/or organic coloring substances have been superimposed.

More particularly, the nacres may have a yellow, pink, red, bronze, orange, brown, gold, and/or coppery color or glint.

Illustrative examples of nacres suitable for being introduced into the composition and that may be mentioned are colored pigments TIMICA SPARKLE GOLD®, CLOISONNE SPARKLE ROUGE 450J®, FLAMENCO SPARKLE GOLD 220J®, FLAMENCO SPARKLE GREEN 820J®, FLAMENCO SPARKLE ORANGE 320J®, FLAMENCO SPARKLE BLUE 620J®, CLOISONNE SPARKLE GOLD 222J®, CLOISONNE SPARKLE GOLD 222J®, CLOISONNE SPARKLE BLUE-ROUGE 650J®, FLAMENCO SPARKLE VIOLET 520J®, CLOISONNE SPARKLE COPPER 350J®, CLOISONNE SPARKLE BRONZE 250J®, DUOCROME SPARKLE BY 226J®, DUOCROME SPARKLE RY 224J/EP 98001®, DUOCROME SPARKLE BR 426J®, DUOCROME SPARKLE RB 624J/EP 98002®, FLAMENCO SPARKLE RED 420J® sold by ENGELHARD, colored pigments TIMIRON DIAMOND CLUSTER MP 149 (17266)® sold by MERCK, and colored pigments KTZ ULTRA SHIMMER® sold by TAIZHU.

Magnetic Bodies

The expression “magnetic bodies” should not be understood in limiting manner and covers particles, fibers, clumps of particles and/or fibers, of any form, presenting non-zero magnetic susceptibility.

The concentration of magnetic bodies in the composition is selected in such a manner as to enable the interference phenomenon to appear in order to create red highlights. The concentration lies in the range about 0.05% to about 50% by weight, for example, in particular in the range about 0.1% to about 40% by weight, better in the range about 1% to about 30% by weight, depending on the kind of magnetic bodies and their incidence on the diffusion of light.

The applied composition may include magnetic fibers or other aspherical bodies, such as chains of particles or of fibers.

In the absence of a magnetic field, the magnetic bodies preferably do not present any remanent magnetism.

The magnetic magnetic bodies may comprise any magnetic material that presents sensitivity to the lines of a magnetic field, regardless of whether the field is produced by a permanent magnet or is the result of induction, the material being selected from nickel, cobalt, iron, and alloys and oxides thereof, in particular Fe₃O₄, and also from gadolinium, terbium, dysprosium, erbium, and alloys and oxides thereof, for example. The magnetic material may be of the “soft” or of the “hard” type. In particular, the magnetic material may be soft iron.

The magnetic bodies may optionally present a multilayer structure including at least one layer of a magnetic material such as iron, nickel, cobalt, and alloys and oxides thereof, in particular Fe₃O₄, for example.

The magnetic bodies are preferably aspherical, presenting an elongate shape, for example. Thus, when the bodies are subjected to the magnetic field, they tend to become oriented with their longitudinal axes in alignment with the field lines, and they are subjected to a change in orientation which results in the composition changing in appearance.

When the magnetic bodies are particles that are substantially spherical, their appearance is preferably non-uniform, so that a change in orientation results in a change in appearance.

Regardless of their shape, the size of the bodies may be in the range 1 nanometer (nm) to 10 millimeters (mm), for example, preferably in the range 10 nm to 5 mm, and more preferably in the range 100 nm to 1 mm, e.g. in the range 0.5 μm to 300 μm or 1 μm to 150 μm.

When the bodies are particles that do not have an elongate shape or that have an elongate shape with a form factor that is fairly small, the size of the particles if less than 1 mm, for example.

The magnetic bodies are magnetic pigments, for example.

Magnetic Pigments

Particularly suitable pigments are nacres comprising iron oxide Fe₃O₄. By way of example, pigments presenting magnetic properties are those sold under the trade names COLORONA BLACKSTAR BLUE, COLORONA BLACKSTAR GREEN, COLORONA BLACKSTAR GOLD, COLORONA BLACKSTAR RED, CLOISONNE NU ANTIQUE SUPER GREEN, MICRONA MATTE BLACK (17437), MICA BLACK (17260), COLORONA PATINA SILVER (17289), and COLORONA PATINA GOLD (117288) by MERCK, or indeed FLAMENCO TWILIGHT RED, FLAMENCO TWILIGHT GREEN, FLAMENCO TWILIGHT GOLD, FLAMENCO TWILIGHT BLUE, TIMICA NU ANTIQUE SILVER 110 AB, TIMICA NU ANTIQUE GOLD 212 GB, TIMICA NU-ANTIQUE COPPER 340 AB, TIMICA NU ANTIQUE BRONZE 240 AB, CLOISONNE NU ANTIQUE GREEN 828 CB, CLOISONNE NU ANTIQUE BLUE 626 CB, GEMTONE MOONSTONE G 004, CLOISONNE NU ANTIQUE RED 424 CHROMA-LITE, BLACK (4498), CLOISONNE NU ANTIQUE ROUGE FLAMBE (code 440 XB), CLOISONNE NU ANTIQUE BRONZE (240 XB), CLOISONNE NU ANTIQUE GOLD (222 CB), and CLOISONNE NU ANTIQUE COPPER (340 XB) by ENGELHARD.

Examples of magnetic pigment suitable for entering into the formulation of the composition that may also be mentioned are black iron oxide particles, e.g. those sold under the trade-name SICOVIT noir E172 by BASF.

The magnetic pigments may also comprise metallic iron, in particular passivated soft iron, e.g. obtained from carbonyl iron by implementing the method described in U.S. Pat. No. 6,589,331, the content of which is incorporated herein by reference. The particles may include an oxide surface layer.

The magnetic bodies may be in the form of flakes.

The size of the magnetic bodies may be less than or equal to 10 μm, or even 1 μm.

The size of the magnetic bodies may also lie in the range 30 μm to 80 μm, thereby making it possible to obtain a pixellization effect that is variable under the effect of the magnetic field, when the red interference pigment presents a size of the same order.

Magnetic Fibers

The term “fibers” means generally elongate bodies presenting, for example, a form factor in the range 3.5 to 2500 or 5 to 500, e.g. 5 to 150. The form factor is defined by the ratio L/D, where L is the length of the fiber and D is the diameter of the circle in which the widest cross-section of the fiber is inscribed.

By way of example, the cross-section of the fibers may be inscribed in a circle having a diameter in the range 2 nm to 500 μm, e.g. in the range 100 nm to 100 μm, or even 1 μm to 50 μm.

By way of example, the fibers may present a length in the range 1 μm to 10 millimeters (mm), e.g. 0.1 mm to 5 mm, or even 0.3 mm to 3.5 mm.

By way of example, the fibers may present a weight in the range 0.15 denier to 30 denier (weight in grams for 9 km of thread), e.g. 0.18 denier to 18 denier.

The cross-section of the fibers may be of any shape, e.g. circular, or polygonal, in particular square, hexagonal, or octagonal.

The composition may contain solid or hollow fibers that may be independent or interlinked, e.g. braided.

The composition may contain fibers having ends that are blunted and/or rounded, e.g. by polishing.

The shape of the fibers need not be significantly modified when they are inserted into the composition, with said fibers being initially rectilinear and sufficiently rigid to keep their shape. In a variant, the fibers may present flexibility that enables them to be substantially deformed within the composition.

The fibers may contain a non-zero amount, that may be as great as 100%, of a magnetic material selected from soft magnetic materials, hard magnetic materials, in particular based on iron, zinc, nickel, cobalt, or manganese, and alloys and oxides thereof, in particular Fe₃O₄, rare earths, barium sulfate, iron-silicon alloys, possibly containing molybdenum, Cu₂MnAl, MnBi, or a mixture thereof, this list not being limiting.

When the composition contains fibers containing magnetic particles, said magnetic particles may be present at least at the surface of the fibers, or only at the surface of the fibers, or only inside the fibers, or they may even be dispersed within the fibers in substantially uniform manner, for example.

By way of example, each fiber may include a non-magnetic core with a plurality of magnetic particles on its surface.

Each fiber may also include a synthetic matrix containing a plurality of magnetic grains dispersed therein.

Where appropriate, a synthetic material filled with magnetic particles may itself be covered by a non-magnetic membrane. By way of example, such a membrane constitutes a barrier isolating the magnetic material(s) from the surrounding environment and/or it can provide color. Each fiber may comprise a one-piece magnetic core and be covered by a non-magnetic membrane, or it may comprise a one-piece non-magnetic core and be covered by a magnetic membrane.

The composition may contain fibers made by extruding or co-extruding one or more polymeric materials, in particular thermoplastics and/or elastomers. One of the extruded materials may contain a filler of dispersed magnetic particles.

Each fiber may comprise a synthetic material selected from polyamides; polyethylene terephthalate (PET); acetates; polyolefins, in particular polyethylene (PE) or polypropylene (PP); polyvinyl chloride (PVC); polyester block amide; plasticized Rilsan®; elastomers, in particular polyester elastomers, polyethylene (PE) elastomers, silicone elastomers, nitrile elastomers; or a mixture of these materials, this list not being limiting.

The composition may contain composite fibers each comprising a magnetic core that is covered, at least in part, by at least one non-magnetic, synthetic, or natural material. By way of example, the magnetic core may be covered by co-extruding a membrane made of a non-magnetic material around the core.

The core may alternatively be covered in some other way, e.g. by polymerization in situ.

The core may be a single piece or it may include a filler of magnetic grains dispersed in a matrix.

The composition may also contain composite fibers obtained by covering a non-magnetic, synthetic, or natural core, with a synthetic material filled with magnetic particles, the core being composed of a fiber made of wood; rayon; polyamide; plant matter; or polyolefin, in particular polyethylene, Nylon®, polyimide-amide, or aramid, this list not being limiting.

The composition may also contain magnetic composite particles, in particular a magnetic latex.

Magnetic Composite Particles

A magnetic composite particle is a composite material constituted by an organic or an inorganic matrix and by magnetic grains. At their surfaces and/or within themselves, the magnetic composite particles may thus include grains of a magnetic material. The composite particles may be constituted by a magnetic core covered by an organic or an inorganic matrix, or they may be constituted by an organic or an inorganic core covered by a magnetic matrix.

The magnetic composite particles include one of the above-mentioned magnetic materials, for example.

The size of the magnetic composite particles may be in the range 1 nm to 1 mm, for example, preferably in the range 100 nm to 500 μm, and more preferably in the range 500 nm to 100 μm. The term “size” means the size given by the statistical grain size distribution at half the population, referred to as “D50”.

The thesis by C. GOUBAULT, dated Mar. 23, 2004, and incorporated herein by reference, refers, in chapter 1, to the prior art on the subject of magnetic composite particles, and draws up a list of preparation methods that are suitable for being used to prepare magnetic composite particles, namely separately synthesizing the magnetic grains and the matrix, synthesizing the magnetic grains in contact with the matrix, or synthesizing the matrix in the presence of the magnetic grains.

KISKER markets inorganic-matrix magnetic composite particles composed of silica. DYNAL, SERADYN, ESTAPOR, and ADEMTECH propose organic-matrix magnetic composite particles that are also suitable for being used in the invention.

More particularly, under the reference M1-070/60, ESTAPOR markets magnetic latex constituted by grains of ferrite that are evenly distributed in a polystyrene matrix, said latex including 65% iron oxide, the mean diameter of the polystyrene particles being 890 nm, and the dry material mass content being 10%.

Ferrofluid

The composition P may contain a ferrofluid, i.e. a stable colloidal suspension of magnetic particles, in particular of magnetic nanoparticles.

The particles, having a size of the order of several tens of nanometers, for example, are dispersed in a solvent (water, oil, organic solvent), either by means of a surfactant or a dispersant, or by electrostatic interactions.

By way of example, the ferrofluids can be prepared by grinding ferrites or other magnetic particles until nanoparticles are obtained, which particles are then dispersed in a fluid containing a surfactant which is absorbed by the particles and stabilizes them, or else they can be prepared by precipitating a metallic-ion solution in a basic medium.

Each particle of the ferrofluid presents a magnetic moment that is determined by the size of the particle, and by the nature of the magnetic material.

Under the action of a magnetic field, the magnetic moments of the particles tend to come into alignment with the field lines, with non-zero magnetization appearing in the liquid. If the field is removed, there is no hysteresis and magnetization drops to zero.

Beyond a field threshold value, it is also possible to cause macroscopic changes in the liquid, e.g. the appearance of peaks, or a change in rheological properties.

The term “ferrofluid” also encompasses an emulsion of ferrofluid droplets in a solvent. Each drop thus contains colloidal magnetic particles in stable suspension. This makes it possible to have a ferrofluid in any type of solvent. The size of the magnetic particles in suspension in the ferrofluid may be in the range 1 nm to 10 μm, for example, preferably in the range 1 nm to 1 μm, and more preferably in the range 1 nm to 100 nm. The term “size” means the size given by the statistical grain size distribution at half the population, referred to as “D50”.

Mention can be made in particular of ferrofluids sold by Liquids Research LTD under the references:

WHKS1S9 (A, B, or C), which is a water-based ferrofluid containing magnetite (Fe₃O₄), having particles of 10 nm in diameter.

WHJS1 (A, B, or C), which is an isoparaffin-based ferrofluid, containing magnetite (Fe₃O₄) particles that are 10 nm in diameter.

BKS25_dextran, which is a water-based ferrofluid stabilized by dextran, containing magnetite (Fe₃O₄) particles that are 9 nm in diameter.

Chains of Particles and/or of Magnetic Fibers

The composition may contain clumps of particles or fibers having a largest dimension, e.g. length, that may, for example, be in the range 1 nm to 10 mm, e.g. in the range 10 nm to 5 mm, or in the range 100 nm to 1 mm, or even in the range 0.5 μm to 3.5 mm, e.g. in the range 1 μm to 150 μm.

By way of example, chains of magnetic particles may be obtained by assembling colloidal magnetic particles, as described in the publications “Permanently linked monodisperse paramagnetic chains”, by E. M. Furst, C. Suzuki, M. Fermigier, A. P. Gast, Langmuir, 14, 7334-7336 (1998), “Suspensions of magnetic particles”, by M. Fermigier, Y. Grasselli, Bulletin of the SFP (105) July 1996, and “Flexible magnetic filaments as micromechanical sensors”, by C. Goubault, P. Jop, M. Fermigier, J. Baudry, E. Bertrand, J. Bibette, Phys. Rev. Lett., 91, 26, 260802-1 to 260802-4 (2003), the contents of which are incorporated herein by reference.

In particular, those articles describe how to proceed in order to obtain chains of magnetic-latex particles that include a polystyrene matrix containing grains of iron oxide with functions on the surface, and that are bonded together in permanent manner following a chemical reaction, in particular covalent bonds between the surfaces of adjacent particles; a method is also described of obtaining chains of ferrofluid-emulsion droplets that are bonded together by physical interactions. The length and the diameter of the permanent chains obtained in this way can be controlled. Such magnetic chains constitute anisotropic magnetic objects that can be oriented and displaced under the effect of a magnetic field.

The dimensions of the magnetic chains may satisfy the same conditions as for the magnetic fibers.

Xchrome Coloring Agent

As mentioned above, the Xchrome coloring agent may be selected so that it takes at least one state in which it generates a color that is red or close to that produced by interference by the red interference pigment or, in contrast, a different color.

The term “color that is close” means that the dominant wavelength is substantially the same, being in the range 580 nm to 650 nm, measured with the above-mentioned imaging calorimeter.

The Xchrome coloring agent may also be selected so that in one state it takes on a color close to that generated by absorption in the surface layer of the interference pigment. This allows the interference pigment to be embedded in the background color to draw an observer's attention to the red highlights when the state of the coloring agent changes.

They may be photochromic coloring agents.

Photochromic Coloring Agents

In general, a photochromic coloring agent is a coloring agent having the property of changing hue when it is illuminated or not illuminated by ultraviolet light and to re-establish its initial color when it is no longer illuminated or is illuminated by a light, or passes from a non-colored state to a colored state and vice versa. In other words, such an agent has different hues depending on whether it is illuminated with light containing a certain quantity of UV radiation.

In the presence of a low level of light, the photochromic coloring agent may take on a substantially non colored state, so that the intensity of the red highlights is not unduly attenuated by the photochromic coloring agent.

In the presence of strong illumination, the photochromic coloring agent may take on a colored state, for example a dark hue or a red color, attenuating the intensity of the red highlights, which may then appear less brilliant than in the presence of low level illumination. This effect may surprise the observer and render the makeup particularly attractive.

The photochromic coloring agent may have a difference ΔE of at least 5. ΔE designates the difference in hue observed in the photochromic substance between its excited state, i.e. in the presence of UV radiation, and its non-excited state, i.e. in the absence of UV radiation.

Reference may usefully be made to examples of photochromic agents described in United States patent application US-A-2004/0228818 the contents of which are hereby incorporated by reference, in particular those with a ΔE of more than 5, as determined using the test presented in this document.

Examples of photochromic coloring agents are naphthopyrane derivatives of the 2H-naphtho-[2,1-b]-pyrane type with formula (I) or 3H-naphtho-[2,1-b]-pyrane type with formula (II): 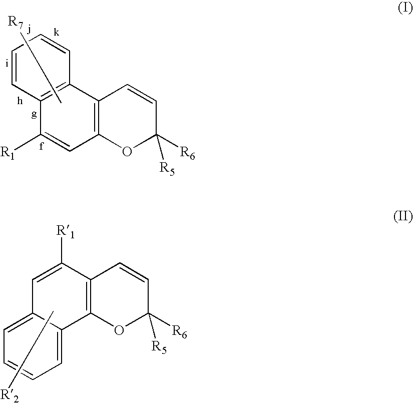
in which:

R₁ represents:

(i) a hydrogen atom;

(ii) a linear, branched, or cyclic, saturated or unsaturated hydrocarbon group containing 1 to 30 carbon atoms, optionally comprising 1 to 5 heteroatoms selected from N, O, S, Si, and P, and/or optionally halogenated or perhalogenated;

(iii) a hydrocarbon cycle formed with one of the “f” or “gh” bonds and the radical R₇; or

(iv) a group selected from —COOR₄, —C(O)NR₂R₃, —NR₂R₃, —OR₄ and —SR₄, in which:

R₂ and R₃ either independently represent a linear, branched, or cyclic, saturated or unsaturated hydrocarbon group containing 1 to 20 carbon atoms, optionally comprising 1 to 5 heteroatoms selected from N, O, S, Si, and P;

or, taken together with the nitrogen atom to which they are bonded, form a saturated or unsaturated hydrocarbon heterocycle containing 3 to 10 carbon atoms and optionally 1 to 5 other heteroatoms selected from N, O, S, Si and P, said cycle optionally being substituted with at least one linear, branched or cyclic, saturated or unsaturated hydrocarbon radical containing 1 to 20 carbon atoms optionally comprising 1 to 5 heteroatoms selected from N, O, S, Si, and P;

R₄ represents a linear, branched or cyclic, saturated or unsaturated hydrocarbon group containing 1 to 20 carbon atoms and/or optionally comprising 1 to 5 heteroatoms selected from N, O, S, Si, and P;

R₅ and R₆ independently represent a group selected from:

(i) saturated cyclic aminoaryl groups with formula (IIA) or (IIB): 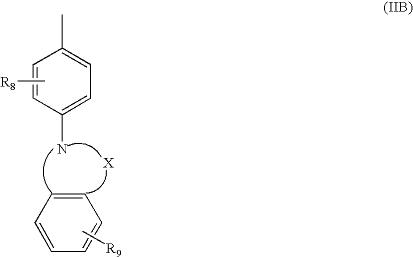
in which the cycle comprising N and X is a saturated cycle which contains a total of 3 to 30 atoms including nitrogen, the remainder being carbon atoms and/or heteroatoms selected from O, S, Si, P, and/or groups selected from —NH and —NR in which R represents a linear, branched, or cyclic, saturated or unsaturated hydrocarbon radical containing 1 to 20 carbon atoms, optionally comprising 1 to 5 heteroatoms selected from N, O, S, Si, and P;

(ii) indolinoaryl groups with formula (III): 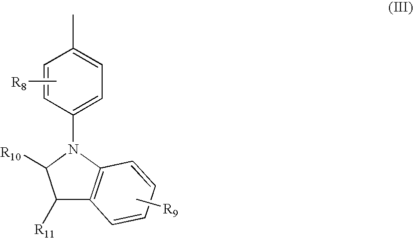
in which R₁₀ and R₁₁ independently represent a group selected from (i) linear, branched, or cyclic, saturated or unsaturated hydrocarbon groups containing 1 to 30 carbon atoms, optionally comprising 1 to 5 heteroatoms selected from N, O, S, Si, and P, and/or optionally halogenated or perhalogenated; (ii) halogen atoms; (iii) —CN (nitrile), —COOH (carboxylate), —NO₂ (nitro) groups; (iv) a hydrogen atom; (v) a group selected from —(O)NR₂R₃, —NR₂R₃, —OR₄ and —SR₄ in which R₂, R₃ and R₄ have the meanings given above; (vi) radicals R₁₀ and R₁₁ may together form a saturated or unsaturated hydrocarbon cycle having a total of 5 to 8 atoms (including the atoms of the indoline cycle), said atoms being selected from C, O, S and/or NR in which R represents H or a linear, branched or cyclic, saturated or unsaturated hydrocarbon radical containing 1 to 20 carbon atoms, optionally comprising 1 to 5 heteroatoms selected from N, O, S, Si, and P;

(iii) groups with formula (IV): 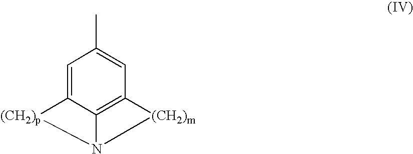
in which m and p are independently integers from 2 to 5;

(iv) unsaturated cyclic aminoaryl groups with formulae (VA), (VB), or (VC):

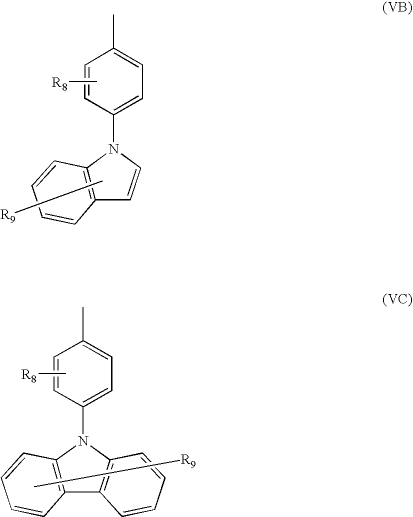

in which R₈ and R₉, independently represent a group selected from (i) linear, branched, or cyclic, saturated or unsaturated hydrocarbon groups containing 1 to 30 carbon atoms, optionally comprising 1 to 5 heteroatoms selected from N, O, S, Si, and P, and/or optionally halogenated or perhalogenated; (ii) halogen atoms; (iii) —CN (nitrile), —COOH (carboxylate), —NO₂ (nitro) groups; (iv) a hydrogen atom; (v) a group selected from —C(O)NR₂R₃, —NR₂R₃, —OR₄, and —SR₄, in which R₂, R₃ and R₄ have the meanings given above;

(v) a linear, branched or cyclic, saturated or unsaturated hydrocarbon group containing 1 to 30 carbon atoms optionally comprising 1 to 5 heteroatoms selected from N, O, S, Si and P; and in particular a group selected from —CONR₂R₃, —C₆H₄—NR₂R₃, and —C₆H₄—OR₄ in which R₂, R₃ and R₄ have the meanings given above;

R₇ represents a group selected from:

(i) linear, branched or cyclic, saturated or unsaturated hydrocarbon groups containing 1 to 30 carbon atoms, optionally comprising 1 to 5 heteroatoms selected from N, O, S, Si, and P, and/or optionally halogenated or perhalogenated;

(ii) halogen atoms;

(iii) —CN (nitrile), —COOH (carboxylate), —NO₂ (nitro); —N═N— (azo); ═NH (imino); —CONH₂ (amide) groups;

(iv) a hydrogen atom;

(v) a group selected from —C(O)NR₂R₃, —NR₂R₃, —OR₄ and —SR₄ in which R₂, R₃ and R₄ have the meanings given above;

(vi) radical R₇ may also form, with one of the “i”, “j”, “k”, or “g,h” bonds taken with radical R₁, or “f” taken with radical R₁, a saturated hydrocarbon cycle containing a total of 3 to 8 carbon atoms, optionally comprising 1 to 5 heteroatoms selected from N, O, S, Si, and P;

R′₁ represents a group selected from:

(i) a hydrogen atom;

(ii) a linear, branched or cyclic, saturated or unsaturated hydrocarbon group containing 1 to 30 carbon atoms optionally comprising 1 to 5 heteroatoms selected from N, O, S, Si, and P, and/or optionally halogenated or perhalogenated;

(iii) a group selected from —C(O)NR₂R₃, —NR₂R₃, —OR₄, and —SR₄, in which R₂, R₃ and R₄ have the meanings given above;

R′₂ represents a group selected from:

(i) linear, branched or cyclic, saturated or unsaturated hydrocarbon groups containing 1 to 30 carbon atoms, optionally comprising 1 to 5 heteroatoms selected from N, O, S, Si and P, and/or optionally halogenated or perhalogenated;

(ii) halogen atoms;

(iii) —CN (nitrile), —COOH (carboxylate), —NO₂ (nitro); —N═N— (azo); ═NH (imino); —CONH₂ (amide) groups;

(iv) a hydrogen atom;

(v) a group selected from —C(O)NR₂R₃, —NR₂R₃, —OR₄ and —SR₄ in which R₂, R₃ and R₄ have the meanings given above.

Further examples of photochromic agents that may be mentioned are diarylethene with formula: 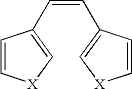
and its derivatives;
dihydroazulene/vinylhepta fulvene, with formula: 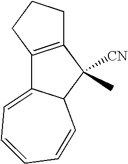
   and its derivatives;
spyronaphthoxazine, with formula: 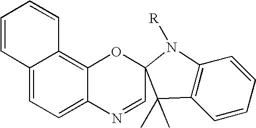
   and its derivatives.

The photochromic agent may be an organic or an inorganic compound. When the photochromic agent is an organic compound, the color change may generally be more rapid and intense.

Examples of photochromic agents that may be mentioned are Photosol® from PPG, which reversibly changes color when activated by UV radiation with a wavelength in the range 300 nm to 360 nm, Reversacol® from J. ROBINSON and Photogenica® from CATALYST & CHEMICALS.

Thermochromic Agents

A thermochromic agent is a pigment or colorant that can change color as a function of temperature.

The thermochromic agent has, for example, a color that is lost when the temperature exceeds a certain value, for example about 15° C. or about 30° C., depending on the nature of the thermochromic agent.

The thermochromic agent may comprise capsules of a polymer containing a solvent, that solvent, depending on whether it is in the molten state or otherwise, allowing compounds to come into contact and modify the light absorption properties.

The color change may be reversible.

As an example, it is possible to use the thermochromic agent sold under the trade name Kromafast® Yellow5GX 02 by KROMACHEM LTD, or Chromazone® as a powder or a dispersion, or Thermobatch® or Thermostar®, from CHROMAZONE.

Piezochromic and Tribochromic Agents

A piezochromic agent can change color in the presence of a mechanical force.

An example of a piezochromic agent that may be mentioned is diphenylflavylene.

A tribochromic agent can change color in the presence of a mechanical force in a manner which is more durable than with piezochromic agents.

Reference may be made to International patent application WO-A-94/26729, the contents of which are hereby incorporated by reference.

Mechanoluminescent Agents

These agents are capable of emitting light when they receive a mechanical stress such as compression, shear, or friction.

The mechanoluminescent agent is preferably in the form of a particle which is insoluble in the cosmetic medium. The mean particle size is, for example, in the range 0.01 μm to 50 μm, preferably in the range 0.1 μm and 10 μm.

Mechanoluminescent materials that may be mentioned are as follows:

a) complexes and chelates of lanthanides such as those described in publications U.S. Pat. No. 6,071,632, US-A-2002/0015965 and WO-A-09/016,429, the contents of which are hereby incorporated by reference. The rare earths are preferably selected from europium, terbium, samarium, and dysprosium. In those materials, diketones are used as the ligand for the trivalent lanthanide salts. These materials are in an organic medium.

b) aluminates, silicates and aluminosilicates doped with rare earth ions such as those described in U.S. Pat. No. 6,280,655, EP-A-0 1 318 184, JP-A-2002/194349, JP-A-2004/59746, the contents of which are hereby incorporated by reference, in particular (Sr, Mg, Ba, Zn, Ca) Al₂O₄, (SrLa, SrY)Al₃O₇, (Sr₂,SrMg,SrCa,SrBa)Al₆O₁₁, Sr₂(Mg,Al)(Al,Si)SiO₇, Sr(Zn,Mn,Fe,Mg)Si₂O₆. The elements shown in parentheses are partially or entirely interchangeable. Rare earth ions such as cerium, europium, samarium, neodymium, gadolinium, dysprosium, and terbium may be used, alone or as a mixture. Europium and dysprosium are preferred;

c) zinc sulfide, manganese sulfide, copper sulfide, cadmium sulfide or zinc oxide, optionally doped with transition metal ions or rare earth ions as described in the publications U.S. Pat. No. 6,117,574 and JP-A-2004/43656 the contents of which are incorporated by reference. Preferred transition metal ions are copper or manganese. Preferred rare earth ions are europium or cerium. Of these materials, ZnS:Mn is preferred.

The materials listed under b) and c) may be synthesized by a solid phase reaction involving dry mixing followed by heat treatment and high temperature sintering, or by a sol-gel process followed by drying, heating and sintering. As an example, the sintering temperature is more than 1000° C.

The materials listed under b) are preferred. Of these, SrAl₂O₄ and SrMgAl₁₀O₁₇ doped with rare metals are preferred.

The mechanoluminescent pigments SrAl₂O₄ doped with rare metal ions are sold with reference TAIKO-Ml-1 by TAIKO Refractories Co., Ltd. The particles of this pigment have a diameter in the range 5 μm to 10 μm and a green luminescence under a weak mechanical stress.

Solvatochromic Agents

A solvatochromic agent can change color in the presence of solvents. DC Red 27 is an example, this compound having an absence of color in an anhydrous formulation; adding water reveals a pink color.

Galenical Forms

The cosmetic composition may be in any galenical form normally used for topical application, in the form of an aqueous solution, an aqueous gel, an oil-in-water or water-in-oil emulsion, a multiple emulsion or a dispersion of oil in water by means of vesicles located at the oil/water interface, on condition that the red overbrightness points are conserved.

The cosmetic composition may constitute, among other makeup products, a liquid lipstick, a liquid gloss, a lipstick paste, a foundation, an eye contour product, a makeup base, a mascara, a nail varnish, an eyeshadow, or a body or hair makeup product.

The composition of the invention may be obtained according to the preparation processes conventionally used in cosmetics.

Conditioning and Modes of Application

The composition may be conditioned in any container or on any support intended for this purpose.

The composition according to the invention may be in the form of a liquid, a paste or a more or less fluid cream.

The composition may be applied using a flocked or non-flocked applicator, for example a foam, a cotton bud, a fine brush, a felt, a spatula, a sinter, a coarse brush, a comb, a woven or a non-woven.

The application may also be performed by finger or by placing the composition directly onto the support to be made up, for example by spraying or projection using a piezoelectric device or by transferring a coat of the composition predeposited onto an intermediate support.

The composition may be applied, for example, to a thickness of between 1 and 10 μm.

The application of the composition is performed, for example, with a mass density of between 1 and 5 mg/cm².

The composition may be applied directly onto the keratin materials or as a top coat over a base coat intended, for example, to constitute a coloured base.

Magnetic Devices

The invention also provides a kit comprising a composition as defined above and at least one magnetic device for generating a magnetic field that makes it possible to displace and/or modify the orientation of the magnetic bodies.

The magnetic device may comprise a permanent magnet or an electromagnet powered by at least one optionally-rechargeable battery, for example. For a battery, the magnetic device may include a switch enabling the electromagnet to be powered selectively with electricity.

The magnetic device may be arranged so as to create a magnetic field of orientation that varies over time. When the magnetic device comprises a magnet, the device may, for example, include a motor enabling the magnet to be rotated. In a variant, the magnetic device may comprise a plurality of solenoids disposed so as to generate a rotating magnetic field when powered sequentially with electricity.

By way of example, a rotating magnetic field may make it possible to obtain a pattern presenting circular symmetry, e.g. a pattern giving the impression of a sphere in relief.

The electromagnet(s) may be powered continuously or intermittently, as desired by the user. In particular, the magnetic device may be arranged so that the electromagnets(s) need not be powered while the magnetic device is not correctly positioned close to the surface coated with the first composition.

The magnetic field is at least 50 milli teslas (mT), for example, and preferably at least 0.2 T, and preferably at least 1 T (10,000 Gauss).

In order to make it easier to apply the magnetic field, the magnetic device may include a member enabling it to be positioned relative to the surface on which the composition has been deposited. This makes it possible to prevent the magnetic device from accidentally coming into contact with the composition and/or makes it possible to center the pattern formed on the region under consideration.

In an implementation of the invention, the magnetic device is secured to an applicator that is used to apply the cosmetic composition. This makes it possible to reduce the number of objects that need to be manipulated by the user and makes it easier to apply makeup.

In another implementation of the invention, the magnetic device comprises a magnet mounted at a first end of a rod having a second end that is connected to a handle of an applicator that is used to apply the cosmetic composition.

The magnetic field may also be exerted by means of a magnetic structure, in particular a flexible structure, including alternate N and S poles. By way of example, such a structure may make it possible to form repeated patterns, e.g. stripes, on the composition.

Makeup Process

A subject of the invention is also a process for making up keratin materials, which consists in applying thereto a composition as defined above.

In another one of its aspects, the invention also provides a makeup method consisting in applying to the keratinous substances, using at least one cosmetic composition, at least one first interference pigment that, when the composition is applied to a surface, is capable of generating red highlights with an intensity that is greater than or equal to 3000 cd·m⁻² and with a dominant wavelength in the range 580 nm to 650 nm; and reflective particles that are capable of generating, on said surface, other highlights with an intensity that is greater than or equal to the intensity of the red interference pigment.

The first interference pigment and the reflective particles can be applied using the same composition.

The first interference pigment and the reflective particles can alternatively be applied using two different compositions that respectively contain the red interference pigment and the coloring agent that is sensitive to at least one external stimulus.

In another one of its aspects, the invention also provides a method of applying makeup to keratinous substances, the method comprising the following steps:

1) applying, to the keratinous substances, a layer of a composition as defined above,

2) subjecting the deposit to a magnetic field, thereby modifying the orientation and/or the position of at least a fraction of the magnetic bodies within the layer deposited in this way.

The present invention also provides a makeup method consisting in using at least one cosmetic composition to apply to the keratinous substances, an interference pigment that is red and that is capable of generating highlights with an intensity that is greater than or equal to 3000 cd·m⁻² and with a dominant wavelength in the range 580 nm to 650 nm, and magnetic bodies that present non-zero magnetic susceptibility.

The red interference pigment and the magnetic bodies can be applied using the same composition.

The red interference pigment and the magnetic bodies can alternatively be applied using two different compositions that respectively contain the red interference pigment and the magnetic bodies.

In another one of its aspects, the invention also provides a makeup method consisting in applying to the keratinous substances, using at least one cosmetic composition, at least one interference pigment that is red and that, once applied, is capable of generating highlights with an intensity of 3000 cd·m⁻² or more and with a dominant wavelength in the range 580 nm to 650 nm; and at least one reflective second pigment that is silvery or colored with a dominant wavelength λ₂ such that |λ₁−λ₂|≦50 nm, this second pigment having an average size that is 30 μm or more, better 40 μm.

The red interference pigment and the reflective second pigment can be applied using the same composition.

The red interference pigment and the reflective second pigment can alternatively be applied using two different compositions that respectively contain the red interference pigment and the reflective second pigment.

In another one of its aspects, the invention provides a a makeup method consisting in applying to keratinous substances, by means of at least one cosmetic composition, at least one red interference pigment that, after application, can generate highlights with an intensity of 3000 cd·m⁻² or more and with a dominant wavelength in the range 580 nm to 650 nm and at least one coloring agent sensitive to at least one external stimulus.

The red interference pigment and the coloring agent which is sensitive to at least one external stimulus may be applied using the same composition.

The red interference pigment and the coloring agent that is sensitive to at least one external stimulus may also be applied via two different compositions respectively containing the red interference pigment and the coloring agent that is sensitive to at least one external stimulus.

Kit

In another one of its aspects, the invention also provides a makeup kit comprising:
a first composition comprising, in a cosmetically acceptable medium, at least one first interference pigment that, when the composition is applied to a surface, is capable of generating red highlights with an intensity that is greater than or equal to 3000 cd·m⁻² and with a dominant wavelength in the range 580 nm to 650 nm; and
a second composition comprising, in a cosmetically acceptable medium, reflective particles that are capable of generating, on said surface, other highlights with an intensity that is greater than or equal to the intensity of the red interference pigment.

In another one of its aspects, the invention also provides a makeup kit comprising:
a first composition comprising, in a cosmetically acceptable medium, an interference pigment that is red and that is capable of generating highlights with an intensity that is greater than or equal to 3000 cd·m² and with a dominant wavelength in the range 580 nm to 650 nm; and
a second composition comprising, in a cosmetically acceptable medium, magnetic bodies that present non-zero magnetic susceptibility.

The second composition may be applied under or over the first.

In another one of its aspects, the invention also provides a makeup kit comprising:
a first composition comprising, in a cosmetically acceptable medium, at least one interference pigment that is red and that, when the composition is applied to a surface, is capable of generating red highlights with an intensity of 3000 cd·m⁻² or more and with a dominant wavelength in the range 580 nm to 650 nm; and
a second composition comprising, in a cosmetically acceptable medium, at least one reflective second pigment that is silvery or colored with a dominant wavelength λ₂ such that |λ₁−λ₂|≦50 nm, this second pigment having an average size that is 30 μm or more, better 40 μm.

In another one of its aspects, the invention provides a a makeup kit comprising:
a first composition comprising, in a cosmetically acceptable medium, at least one red interference pigment that, when the composition is applied to a support, can generate highlights with an intensity of 3000 cd·m⁻² or more and with a dominant wavelength in the range 580 nm to 650 nm;
a second composition comprising, in a cosmetically acceptable medium, at least one coloring agent which is sensitive to at least one external stimulus.

### EXAMPLES PROPOSED

The contents indicated are on a mass basis.

### Example 1

### Blusher

| | |
|---|---|
| Triethanolamine | 1 |
| Disodium ethylenediaminetetraacetate dihydrate | 0.2 |
| Crosslinked carboxyvinyl homopolymer | 0.5 |
| Polyvinylpyrrolidone | 0.6 |
| Glycerol | 5.75 |
| Deionized water | 83.05 |
| 1,3-Butylene glycol | 2 |
| Silica microspheres (3 μm) | 1.5 |
| Red interference pigment* | 5 |

*Pigment comprising a silica core coated with a layer of iron oxide Fe₂O₃, available from the company Merck under the reference Xirona Red.

A very bright makeup of sparkling bright red is obtained.

### Example 2

### Nail Varnish

| | |
|---|---|
| Tetrasodium pyrophosphate | 0.2 |
| Oxyethylenated polydimethylsiloxane with methoxy end groups | 0.5 |
| Mixture of aliphatic polyurethane, N-methylpyrrolidone, | 75 |
| triethylamine and water (35/8.5/2/54.5) | |
| Glycerol | 1 |
| Deionized water | 15 |
| Ethyl alcohol (96°) | 2.8 |
| Synthetic Laponite (mixed magnesium/lithium/sodium silicate) | 1.3 |
| Red interference pigment* | 4.2 |

*idem Example 1.

Substantially the same effect as that of Example 1 is obtained.

Needless to say, the invention is not limited to the examples that have just been given. The term “comprising one” is synonymous with “comprising at least one”, and “between” is understood as meaning limits inclusive.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A cosmetic composition comprising
a cosmetically acceptable medium containing at least one aqueous phase,
at least one interference pigment dispersed in this aqueous phase, capable of generating, when the composition is applied to a support, overbrightness points with an intensity of greater than or equal to 3500 cd·m⁻² and with a dominant wavelength of between 580 and 650 nm.

2. A composition according to claim 1, the intensity being greater than or equal to 4200 and better still 4500.

3. A composition according to claim 1, the medium having a mass content of water of greater than or equal to 10%.

4. A composition according to claim 3, the medium having a mass content of water greater than or equal to 25%.

5. A cosmetic composition according to claim 1, the content of red interference pigment being less than or equal to 10%.

6. A cosmetic composition according to claim 1, the content of red interference pigment being greater than or equal to 3%.

7. A composition according to claim 1, the red interference pigment being the only colouring agent present in the composition.

8. A composition according to claim 1, the size of the red interference pigment being greater than or equal to 30 μm.

9. A composition according to claim 8, to size of the red interference pigment being greater than or equal to 40 μm.

10. A composition according to claim 1, the red interference pigment comprising an inorganic core.

11. A composition according to claim 10, the core being made of silica, glass or mica.

12. A composition according to claim 1, comprising a surface layer of a metal oxide.

13. A composition according to claim 12, the metal oxide comprising iron oxide Fe₂O₃.

14. A cosmetic composition according to claim 1, constituting a blusher.

15. A cosmetic composition according to claim 1, constituting a nail varnish.

16. A cosmetic composition according to claim 1, the medium comprising a film-forming agent in a mass content ranging from 1% to 90%.

17. A composition according to claim 1, the red interference pigment being capable of creating highlights with any intensity of 3500 cd·m⁻² or more, the composition not containing, in the medium, white fillers or solid bodies that generate a color by absorption, or, when the composition does contains them, the total amount of such solid bodies being 1% or less by weight relative to the total weight of the composition.

18. A composition according to claim 1, comprising reflective particles that are capable of generating, on said surface, other highlights with an intensity that is greater than or equal to the intensity of the red interference pigment.

19. A composition according to claim 1, presenting a turbidity index of 100 NTU or less.

20. A composition according to claim 1, comprising magnetic bodies presenting non-zero magnetic susceptibility.

21. A composition according to claim 1, comprising a reflective second pigment that is silvery or that is colored with a dominant wavelength λ₂ such that |λ₁−λ₂|≦50 nm, this second pigment having an average size that is 30 μm or more.

22. A composition according to claim 1, comprising at least one coloring agent that is sensitive to at least one external stimulus.

23. A set of at least two cosmetic compositions according to claim 1, the saturation difference between two compositions of the set being 2 or less, the red interference pigment in said two compositions being at concentrations that differ by at least 1%.

24. Process for making up keratin materials, comprising applying thereto a composition as defined in claim 1.
